SEQUENCE LISTING (222 sequences)

SEQ ID NO: 1 - DNA, 456 bp, synthetic construct
  source 1..456: mol_type = other DNA
atggctagcatgactggtggacagcaaatgggtcgcggatccgttggtaaaaccatttggctgcagggctttaacaacaaatatgtgaatagcaaaaatggccagggtgcaatgtggtgtgatagtgatgcaccgcaggcatgggaactgtttaccgttgttgatgcaggtaatggtaaaattgcactgcgtggtaataatggcatgtatgttagcagcgaaaatggtgaacaggcgattacctgtaatcgtccggcaattcaaggttgggaagcatttgattggctggaaaccgcagatggtaaagtgagcctgcgtggttcaaatggtctgtttattagctcagaaaatggcgcagcagcaatgacctgtacacgtccgaccgcaagcggctgggaagcctttggttatagcgttgttggtaatgcactcgagcaccaccaccaccaccactga

SEQ ID NO: 2 - DNA, 423 bp, synthetic construct
  source 1..423: mol_type = other DNA
atgccgatcggtaaaacgatctggttacagggctttaacagcaaatacgttaattcaagaaacggacagggtgctatgtggtgtgattccgatactccgcaggcatgggaactctttactgtcatagatgcgggtaacggtaagatcgcgctgagaggtaacaacggtctgtatgtttcttctgaaaatggcgaacaggcaatgacctgcaacagacctgctattgacggatgggaagtattcgactggatcagcaacagtgatggttccgtttccctgcgtggcagcaacggaatgtatgtttcctccgaaaatggtgagcaggcaattacttgtaacagacctgctattgacggatgggaaagatttaactgggcagcagcaacagcattgactggccatcaccatcaccaccacggctaa

SEQ ID NO: 3 - protein, 150 aa, synthetic construct
MASMTGGQQMGRGSVGKTIWLQGFNNKYVNSKNGQGAMWCDSDAPQAWELFTVVDAGNGKIALRGNNGMYVSSENGEQAITCNRPAIQGWEAFDWLETADGKVSLRGSNGLFISSENGAAAMTCTRPTASGWEAFGYSVVGNALEHHHHH

SEQ ID NO: 4 - protein, 140 aa, synthetic construct
MPIGKTIWLQGFNSKYVNSRNGQGAMWCDSDTPQAWELFTVIDAGNGKIALRGNNGLYVSSENGEQAMTCNRPAIDGWEVFDWISNSDGSVSLRGSNGMYVSSENGEQAITCNRPAIDGWERFNWAAATALTGHHHHHHG

SEQ ID NO: 5 - protein, 151 aa, synthetic construct
MASMTGGQQMGRGSVGKTIWLQGFNNKYVNSKNGQGAMWCDSDAPQAAELFTVVDAGNGKIALRGNNGMYVSSENGEQAITCNRPAIQGWEAFDWLETADGKVSLRGSNGLFISSENGAAAMTCTRPTASGWEAFGYSVVGNALEHHHHHH

SEQ ID NO: 6 - protein, 151 aa, synthetic construct
MASMTGGQQMGRGSVGKTIWLQGFNNKYVNSKNGQGAMWCDSDAPQAWELFTVVDAGNGKIALRGNNGMYVSSENGEQAITCNRPAIQGAEAFDWLETADGKVSLRGSNGLFISSENGAAAMTCTRPTASGWEAFGYSVVGNALEHHHHHH

SEQ ID NO: 7 - protein, 151 aa, synthetic construct
MASMTGGQQMGRGSVGKTIWLQGFNNKYVNSKNGQGAMWCDSDAPQAWELFTVVDAGNGKIALRGNNGMYVSSENGEQAITCNRPAIQGWEAFDWLETADGKVSLRGSNGLFISSENGAAAMTCTRPTASGAEAFGYSVVGNALEHHHHHH

SEQ ID NO: 8 - protein, 33 aa, synthetic construct
IWLQGFNNKYVNSKNGQGAMWCDSDAPQAWELF

SEQ ID NO: 9 - protein, 33 aa, synthetic construct
IWLQGFNSKYVNSRNGQGAMWCDSDTPQAWELF

SEQ ID NO: 10 - protein, 33 aa, synthetic construct
IALRGNNGMYVSSENGEQAITCNRPAIQGWEAF

SEQ ID NO: 11 - protein, 33 aa, synthetic construct
IALRGNNGLYVSSENGEQAMTCNRPAIDGWEVF

SEQ ID NO: 12 - protein, 33 aa, synthetic construct
VSLRGSNGLFISSENGAAAMTCTRPTASGWEAF

SEQ ID NO: 13 - protein, 33 aa, synthetic construct
VSLRGSNGMYVSSENGEQAITCNRPAIDGWERF

SEQ ID NO: 14-16 - skipped (3 entries)

SEQ ID NO: 17 - protein, 151 aa, synthetic construct
MASMTGGQQMGRGSVGKTIWLQGFNNKYVNSKNGQGAMWCDSDAPQAWELFTVVDAGNGKIALRGNNGMYVSSENGEQAITCNRPAIQGAEAFDWLETADGKVSLRGSNGLFISSENGAAAMTCTRPTASGAEAFGYSVVGNALEHHHHHH

SEQ ID NO: 18 - protein, 151 aa, synthetic construct
MASMTGGQQMGRGSVGKTIWLQGFNNKYVNSKNGQGAMWCDSDAPQAAELFTVVDAGNGKIALRGNNGMYVSSENGEQAITCNRPAIQGWEAFDWLETADGKVSLRGSNGLFISSENGAAAMTCTRPTASGAEAFGYSVVGNALEHHHHHH

SEQ ID NO: 19 - protein, 151 aa, synthetic construct
MASMTGGQQMGRGSVGKTIWLQGFNNKYVNSKNGQGAMWCDSDAPQAAELFTVVDAGNGKIALRGNNGMYVSSENGEQAITCNRPAIQGAEAFDWLETADGKVSLRGSNGLFISSENGAAAMTCTRPTASGAEAFGYSVVGNALEHHHHHH

SEQ ID NO: 20 - protein, 129 aa, synthetic construct
VGKTIWLQGFNNKYVNSKNGQGAMWCDSDAPQAWELFTVVDAGNGKIALRGNNGMYVSSENGEQAITCNRPAIQGWEAFDWLETADGKVSLRGSNGLFISSENGAAAMTCTRPTASGWEAFGYSVVGNA

SEQ ID NO: 21 - protein, 131 aa, synthetic construct
PIGKTIWLQGFNSKYVNSRNGQGAMWCDSDTPQAWELFTVIDAGNGKIALRGNNGLYVSSENGEQAMTCNRPAIDGWEVFDWISNSDGSVSLRGSNGMYVSSENGEQAITCNRPAIDGWERFNWAAATALT

SEQ ID NO: 22 - protein, 133 aa, synthetic construct
IANRENISIRALSNNLYVSSENGLRPITCNRTKVDIWERFSIESVGGGRVAIKGSNGSYFSSEDGLKPMRCNRKKAEAWEEFTLEPLGGDLYAIKGNNGAYVSHNNGSVEGLTCNKSIVGDQEKFIIKDAASK

SEQ ID NO: 23 - protein, 141 aa, synthetic construct
TTDAPIGKTISLRKTNGDKKYVTAEQTANNKQLIARAIAVQGWEKFSIETHPDGGIALKALSNANYVQVQGINVNAAAAKKDKLTQFIWKNKGNGKVALKSVSANKWIQASWSSDNAVLFAKGIEDKGWETFDWKIENTQK

SEQ ID NO: 24 - protein, 127 aa, synthetic construct
TGPIGQSVTLKGFNNQYVSSENGTQAMNCNRPTASGWETFSVVDAGGGKIALLSQGKYVSSENGTQAMTCNRLTIGDWEKFDWVVNADGKISLRGSNGQYVSSENGTQPMNCNRATISGWEAFGVNQ

SEQ ID NO: 25 - protein, 129 aa, synthetic construct
PVGQIITLRGSNNLYVSGENGTKAMTCERTAPQTWEQFSVWNAGPGKVNLRSMGKYVSSENGLQAITCNRTTAASYEAFDWISNADGTVSLRGNNGLYVSSENGAAAMTCTRPTIDGWEKFNFTIVGPA

SEQ ID NO: 26 - protein, 127 aa, synthetic construct
GGTAPIGSTITIKGSNGLFASGENGAQAMTCNRPTAQAWEQFTVVDAGGGKVALRSQGMYVSSENGAQAMTCSRPTIQDWEKFNWIDNGDGTFSLRGNNGSYVSSENGTQAMTCNRPTIQGWEKFTR

SEQ ID NO: 27 - protein, 124 aa, synthetic construct
IGQTVTIKGFNNQYVCSEGNTQPMICNRAVAQSWEQFTVVDAGGGRVALRNQGNYVCSENGTQAVNCNRASVGPWEQFEWISNSDGTISFRGNNGAYLSAEDGMARMTCTKTTIGAAEKFKINQ

SEQ ID NO: 28 - protein, 125 aa, synthetic construct
VGSIIYLYHDTLLVCSENGTQAMNCNRTGLGPWEKFEVVDAGNNTVALKGNNGLYVKAGNPVFCTGTALDSSTCFNWISLSSNTVALQSGKGLYMSSENGTQAMNWNRTAIGGWETFRWGTTTAA

SEQ ID NO: 29 - protein, 136 aa, synthetic construct
PSIDIDEGKIALKSVHGKYLSAQPDGRAEWNRNIASEWEYFHLEKRQGDKITLKGAHGMYVSAQPDGEVQINRQAAPPTGWEEFTVEDRGNNVICLKSIHWKYLSAQMDGTVQWNRDSAPKGGWEEFQIVRPGGPG

SEQ ID NO: 30 - protein, 128 aa, synthetic construct
SGAPIGSTITLKGFNNQYVSSENGTQAMNCNRPTASAWEQFLVVDAGAGKIALQSMGKYVSSENGTQAITCNRTTYGDWEKFDWVPTTDGKVTLRGNNGKFISSENGTQAMTCTRATASGWEAFGVNQ

SEQ ID NO: 31 - protein, 128 aa, synthetic construct
PAPPIGTVISLKGFNGKYVSGENGTQAMTCNRTVAGDWEHFTVLDAGNGKIYLRSMGKYVSSENGTQAITCNRTTPSDWEKFDWIVTTDGKITLRGNNGKFISSENGTQAMTCNRTTASGWEAFGLNQ

SEQ ID NO: 32 - protein, 127 aa, synthetic construct
TPPIGQTITLKGSNNNYVSSENGTQPMNCNRPTAGGWEQFTIVNAGSGKVALLNSGKYVSSENGTQAINCNRTSVGPWEQFDWVGNADGTVSFRGSNGKYISGENGTQAMTCNRATIGGWESFRVNQ

SEQ ID NO: 33 - protein, 144 aa, synthetic construct
AVAAPVGQTIWLKACSTQQFVSADQNLGATAPLVANRATVQGWEQFQVVDAGGGTIALRATGSGLYVSADTNVGGQLTANRPTIQDWERFEWVELGNGSIGLKARSNGLYVSADLGRNASAPLYASRASIGGCWEAFTWGSVGG

SEQ ID NO: 34 - protein, 34 aa, synthetic construct
ISIRALSNNLYVSSENGLRPITCNRTKVDIWERF

SEQ ID NO: 35 - protein, 33 aa, synthetic construct
VAIKGSNGSYFSSEDGLKPMRCNRKKAEAWEEF

SEQ ID NO: 36 - protein, 34 aa, synthetic construct
YAIKGNNGAYVSHNNGSVEGLTCNKSIVGDQEKF

SEQ ID NO: 37 - protein, 37 aa, synthetic construct
ISLRKTNGDKKYVTAEQTANNKQLIARAIAVQGWEKF

SEQ ID NO: 38 - protein, 32 aa, synthetic construct
IALKALSNANYVQVQGINVNAAAAKKDKLTQF

SEQ ID NO: 39 - protein, 36 aa, synthetic construct
VALKSVSANKWIQASWSSDNAVLFAKGIEDKGWETF

SEQ ID NO: 40 - protein, 33 aa, synthetic construct
VTLKGFNNQYVSSENGTQAMNCNRPTASGWETF

SEQ ID NO: 41 - protein, 32 aa, synthetic construct
IALLSQGKYVSSENGTQAMTCNRLTIGDWEKF

SEQ ID NO: 42 - protein, 33 aa, synthetic construct
ISLRGSNGQYVSSENGTQPMNCNRATISGWEAF

SEQ ID NO: 43 - protein, 33 aa, synthetic construct
ITLRGSNNLYVSGENGTKAMTCERTAPQTWEQF

SEQ ID NO: 44 - protein, 32 aa, synthetic construct
VNLRSMGKYVSSENGLQAITCNRTTAASYEAF

SEQ ID NO: 45 - protein, 33 aa, synthetic construct
VSLRGNNGLYVSSENGAAAMTCTRPTIDGWEKF

SEQ ID NO: 46 - protein, 33 aa, synthetic construct
ITIKGSNGLFASGENGAQAMTCNRPTAQAWEQF

SEQ ID NO: 47 - protein, 32 aa, synthetic construct
VALRSQGMYVSSENGAQAMTCSRPTIQDWEKF

SEQ ID NO: 48 - protein, 33 aa, synthetic construct
FSLRGNNGSYVSSENGTQAMTCNRPTIQGWEKF

SEQ ID NO: 49 - protein, 33 aa, synthetic construct
VTIKGFNNQYVCSEGNTQPMICNRAVAQSWEQF

SEQ ID NO: 50 - protein, 32 aa, synthetic construct
VALRNQGNYVCSENGTQAVNCNRASVGPWEQF

SEQ ID NO: 51 - protein, 33 aa, synthetic construct
ISFRGNNGAYLSAEDGMARMTCTKTTIGAAEKF

SEQ ID NO: 52 - protein, 32 aa, synthetic construct
IYLYHDTLLVCSENGTQAMNCNRTGLGPWEKF

SEQ ID NO: 53 - protein, 30 aa, synthetic construct
VALKGNNGLYVKAGNPVFCTGTALDSSTCF

SEQ ID NO: 54 - protein, 33 aa, synthetic construct
VALQSGKGLYMSSENGTQAMNWNRTAIGGWETF

SEQ ID NO: 55 - protein, 32 aa, synthetic construct
IALKSVHGKYLSAQPDGRAEWNRNIASEWEYF

SEQ ID NO: 56 - protein, 34 aa, synthetic construct
ITLKGAHGMYVSAQPDGEVQINRQAAPPTGWEEF

SEQ ID NO: 57 - protein, 34 aa, synthetic construct
ICLKSIHWKYLSAQMDGTVQWNRDSAPKGGWEEF

SEQ ID NO: 58 - protein, 33 aa, synthetic construct
ITLKGFNNQYVSSENGTQAMNCNRPTASAWEQF

SEQ ID NO: 59 - protein, 32 aa, synthetic construct
IALQSMGKYVSSENGTQAITCNRTTYGDWEKF

SEQ ID NO: 60 - protein, 33 aa, synthetic construct
VTLRGNNGKFISSENGTQAMTCTRATASGWEAF

SEQ ID NO: 61 - protein, 33 aa, synthetic construct
ISLKGFNGKYVSGENGTQAMTCNRTVAGDWEHF

SEQ ID NO: 62 - protein, 31 aa, synthetic construct
YLRSMGKYVSSENGTQAITCNRTTPSDWEKF

SEQ ID NO: 63 - protein, 33 aa, synthetic construct
ITLRGNNGKFISSENGTQAMTCNRTTASGWEAF

SEQ ID NO: 64 - protein, 33 aa, synthetic construct
ITLKGSNNNYVSSENGTQPMNCNRPTAGGWEQF

SEQ ID NO: 65 - protein, 32 aa, synthetic construct
VALLNSGKYVSSENGTQAINCNRTSVGPWEQF

SEQ ID NO: 66 - protein, 33 aa, synthetic construct
VSFRGSNGKYISGENGTQAMTCNRATIGGWESF

SEQ ID NO: 67 - protein, 37 aa, synthetic construct
IWLKACSTQQFVSADQNLGATAPLVANRATVQGWEQF

SEQ ID NO: 68 - protein, 35 aa, synthetic construct
IALRATGSGLYVSADTNVGGQLTANRPTIQDWERF

SEQ ID NO: 69 - protein, 35 aa, synthetic construct
IGLKARSNGLYVSMEGGSSPINANRASIGGCWEAF

SEQ ID NO: 70 - protein, 133 aa, synthetic construct
SYIGIGDNVWFEAYNGSFISSENGASPMTCNTFTVGETEVFTIVDAGDGKIALLGNNGKYVSSNNGTTSMTCTKDEIGETEKFYWINLSNGQMALLGKGGFVSMEGGSSPINANRNAIDGWEIYSWGKQDIQT

SEQ ID NO: 71 - protein, 147 aa, synthetic construct
SAAAPVGQTIWIRASATGDYVSADQNRGSFAPLVADRAAANSWEQFQVVDAGGGYIALKSVGTGLYVSADLNRGAFAPLVADRPSADTWEHFIWTDLTGSAFTLRGRVTGHYVSADLNRGDFAPLVSDRVAASTWETFTWGAVGGTT

SEQ ID NO: 72 - protein, 143 aa, synthetic construct
GLAAPIGRNIALRACSTQQFVSADQNLANTALVANRATAQGWEQFQVVDAGGGTVALRSVSTGLFVSADTNLGGQLVANRNAIQDWERFEWVEFGGDSVGLKARSNGLFVSADTSLGASGPLYANRAAAGCWESFSVSVIGGG

SEQ ID NO: 73 - protein, 144 aa, synthetic construct
GLAAPLGRNITLRACSTQQFVSADQNLANTALVANRATAQGWEQFQVVDAGGGLVALRSVSTGLFVSADTNLGGRLVANRTSIQDWERFEWVDLGGDAVGLKARSNGLFVSADLNQGASGPLFANRASAAGCWESFSVSVVGGG

SEQ ID NO: 74 - protein, 144 aa, synthetic construct
AVAAPVGQTIWLRACASQQYVSADQNLGATAPLVANRATVQGWEQFQVVDAGSGSIALRATGSGLYVSADTNLGGQLVANRPGIQDWERFEWVELGNGSIALKARSNGLFVSADLERGASAPLYASRAAVGGCWESFTWAAIGG

SEQ ID NO: 75 - protein, 142 aa, synthetic construct
LAAAPIGQTIWLKACTNQQYVSADQNINANTPLVANRATAQGWEQFQVKDGGSGQVALLANSNGKYVSADPNANGQLTASKTTVGDWERFQWVSQADGTVSLLAKSTAKYVAADSNLANTQLVANRAATDCWERFSWGAVGG

SEQ ID NO: 76 - protein, 144 aa, synthetic construct
ATAAPLGRTIWLRACATQKYVSADRNIHANAPLVANRDTFQGWEQFVVGDAGNDFISLRVSETGLYVSADPNASGQVTGFRTAVGDWERFTWVPFPDGTVALKAKSNNLFVSADANQGGTAPLYANRTTAGCWEAFSFGIVGGG

SEQ ID NO: 77 - protein, 144 aa, synthetic construct
ATAAPLGRTVWLRACATQKYVSADKNLNATAPLVANRDTFQGWEQFLVGDAGNDFISLRVSETGLYVSADPNAGGQVTGFRTAVGDWERFTWVPFPDGTVALKAKSTNLFVSADANQGGTAPLYANRATAGCWEAFSFGIVGGG

SEQ ID NO: 78 - protein, 145 aa, synthetic construct
LAAAPIGQTIWLKACSTQKYVSADKNISADAPLVADRAAAEGWEQFQVGDAGNGYITLRVAETGLYVSADTNLGGKLVANRASPGDWEHFQWVDYGNGSIGLKARSNGLFVTSDLNQGAAGPLVASRAASAGCWESFTWAAVGGG

SEQ ID NO: 79 - protein, 143 aa, synthetic construct
PLAAPIGQTIWLKACLNSKFISADGNLGATAPLVADRAAAAGWELFQIVDAGSGTIALRVSETGMYVSADTNLGGQLVANRTTIGDWERFTWVDFNNGTYGLIAKSTGKYVSTDSNRGANAPLYADRATAGCWESFSFGTSGS

SEQ ID NO: 80 - protein, 145 aa, synthetic construct
SVKAPIGQTIWLKSCLTGKFVSADGNLGANSPLVADRATTAGWEHFQVVDAGSGTIALRVSETQKYVSADTNVAGRLVADRTTIGDWERFTWVEFADGNIGLIAKSTGKYVSTDTNRGANAPLYADRTTAGCWEAFSIGIVGGGG

SEQ ID NO: 81 - protein, 135 aa, synthetic construct
NTNVNGTVTIKGQSINKYVSSEGGFGPMRANYTQAGNTEQFTVAPAGNGTISLKGNNGKYVSSENGKKAMTCNRNAVGAWEKFRLESLGGDLYAIKGNNNKYVSHENGKNTGITCNRNAIGSWEKFIITGLNNKS

SEQ ID NO: 82 - protein, 129 aa, synthetic construct
TVTMKGQSINKFISSENGNRSMRAVSNSAGGQEKFIVKSEGNGTVSFKGNNGKYVSSEDGKKAMNCNRNVVGAWEKFTLVPQGGNVYAIRGNNGKYVSHENGSNNGINCNRNAIGSWEKFVINGLNKSK

SEQ ID NO: 83 - protein, 125 aa, synthetic construct
ADVQMASLQNQGSFNFMSATDPNKMSNKATAGTTEQFQIIDIGDGKVALKGSNGKYVSSENGTKEMTCTRTEIGAWEKFTLIDYGNNIYALKGNNNLYLRDNMLCTSTGVSDWQRFKITYNNSNA

SEQ ID NO: 84 - protein, 128 aa, synthetic construct
SVSTGSLSSFQNQGSFKYMSSADINKLTCNTTTVGTSEKFEIVDIGSGLVALKGSNGKYVSSENGTKELTCTRTSIGAWEKFTMVDYGNNVYALKGNNDLYVRDNMLCTSTGASNWQQFKVTSELKNA

SEQ ID NO: 85 - protein, 125 aa, synthetic construct
SGNLTSFQNQGSFKYMSSSNINKLACNKSIAGSTEKFEIIDIGSGLVALKGSNGKYVSSENGTKELTCTGTSIGAWEKFTMVDYGNNVYALKGNNNLYVRNNMLCTSNGVSNWQQFKVTSGLKNA

SEQ ID NO: 86 - protein, 133 aa, synthetic construct
VFRGIDKYVWFQAYNGTYVSSKGGDVPMTCNSVGLGDQEIFKIVDANDGQIALQGNNGKYVSSNNGMTSMTCSKSEIGESEKFYWINLPNHEVALLGLGGFVSMEDGSIPMNANRRNFDGWEIFTWGTKDVAT

SEQ ID NO: 87 - protein, 133 aa, synthetic construct
PYRGIGDYISFEAYNYTYISSESGNSAMTCSSTSLTDDKYFLVVDAGDGMIALKGNNGKYVSSNNGTSSMTCDKDEIGENEKFYWIDLASGKMALLGKGGFVSMEGGSIPINANRTGIDGWEVYYWGKKDIST

SEQ ID NO: 88 - protein, 133 aa, synthetic construct
ISKPTVGKFIRMKAYNGNYLSYSATDQQLTISSSQPGARAWFQLVDAGDGLVALKTLDDQYVSVQTDGNSMTANSSVIGEAEQFTFINLGNGRMALEGMQDFFVSAENGSLPVACNRTYIDGYEIFEWEEVSN

SEQ ID NO: 89 - protein, 135 aa, synthetic construct
VQTLPIGQTVWLQGSNLKYVSSEGNTNPMTCTRDSYDGWELFEIVDAGDGKVALLGSNNYYVNSQNGENSMMCNTTAIGEWEKFSFVQFTDNQIALYGNNERFVSSEDGLQAMNCNRESIGGWEVFNWGNSSTSS

SEQ ID NO: 90 - protein, 135 aa, synthetic construct
TSVPPVGRTIWLKGNNDKYVSSEGNDNPMTCTRTTYGGWELFTIVDAGGGKIALKGSNNLYVSSQNGTAAMICNKTAIGETEKFTFINIGDWKVALRGTNGKYVNSENGSNPMMCDKPEIAVWESFTWGDGISGA

SEQ ID NO: 91 - protein, 134 aa, synthetic construct
IPSKPIGSYIWLKNSNKYVSSNNGSGPMTANRNGVGGWEKFQVISAGDTKIALKGNNNKYVNSQNGNSPLTCTSNSVSGWESFEWIEINGQVALKGFNGKFVSAEGGSSNGMNSNRETASGWEVFSWGTTSASR

SEQ ID NO: 92 - protein, 134 aa, synthetic construct
LPPQPIGRYIWLRNSNKYVSSNNGSGPVTCDRSSRGQWERFRVVSAGDRKIALKGNNRRYINSQNGNSAMTCTSTSVQGWESFEWIDVYGNVALKGFNGKYVSSEGGSSNGMNSNRDTASGWEVFEWQDLSKNG

SEQ ID NO: 93 - protein, 134 aa, synthetic construct
PPVPTPGSRVALQASNGQYVCAEGGGGQAVVANRNAIGPWETFTLKDLGNNKIALQASNGYFVCAEGGGGQAVVANRNAIGPWETFTLKDLGNNKIALQASNGNFVCAEGGGGQAVVANRNAIGSWETFTRLNS

SEQ ID NO: 94 - protein, 133 aa, synthetic construct
SGAAPIGSTVWLQGNNGKFVSSEDGLAPMMCDRDSVQGWESFAVVDAGGGTVALRCSNGAYVSSEDGLAPITCNRTAIGGWERFVWEPTNDGKVALRGSNGCYVSSEDGAAAMTCNRSSVSGWEAFAWGTVGG

SEQ ID NO: 95 - protein, 129 aa, synthetic construct
GSGAPIGQTITLKGFNGQYVSGENGTQAMNCNRPTAQAWEQFLVVDAGGGKIALQSMGKYVSSENGVNPITCNRATVGAWEQFTWIVNADGTIALQGNNGRYISSENGAQAMTCNRTAISGWEAFGVNQ

SEQ ID NO: 96 - protein, 129 aa, synthetic construct
STSAPIGSVVTLKGFNNQYVNSQNGTQAMTCSSAVASTWEQFTVVDAGGGKIALRAMNKYVSSENGTQAITCSRTTIGDWEKFDWVPTADGKVTFRGSNGKYISSENGTQPMTCSRATASGWEAFAINQ

SEQ ID NO: 97 - protein, 134 aa, synthetic construct
NSEAPIGKIITLKNNKAYVSSENGSKAMTCFRATAGDWEKFKVVDAGEGKIALKGNNNKYVSAENGINRMTCNKSTIGHTEKFKWEELNTNTVVLRGSNGKNVSSAGNKNPMICNLTTIGPWSKFIWTVPTISN

SEQ ID NO: 98 - protein, 135 aa, synthetic construct
GSGVPIGQTIWLRGTNGKFVSSENGGTGMMCNRNSVGGWEQFTVVDAGGGKIGLRGTNGLYVSSENGASAMMCNRGSIGGWEAFDWVDTGNGNIALRGTNGQYVSSEDGVSPMMCNRSSIDGWENFTYGIVGSNT

SEQ ID NO: 99 - protein, 133 aa, synthetic construct
MYPSNSNKVITFKGNNDRYISSEDGKRTLTINRTRVGAFERFELIELENGKVAFKGNNGKYISSENGTKSMICNRENIGSWEQFTLISKGRNNYNIRGNNGKYISSNGGDDSKGVACDRSSARSFELFTISGL

SEQ ID NO: 100 - protein, 135 aa, synthetic construct
AGDNPSTWRITIQGSNGKYLSSEDGLSAMMCDRDEADDWEQFDVIDNPDGTFSLRGSNGKYVSSENGNSPMMCDRNTIDDWEKFNWVELGGNAFAIKGSNGNYVSSENGLSGVMCNRPDIGEWETFYWTTVASGA

SEQ ID NO: 101 - protein, 127 aa, synthetic construct
STTQKVSLKNQNNNLYVCSENGLQEMTCNRSSVGDWEKFEFIDLGNSVFALKGNNGLYVSSENGDKAMTCTRTDIGAWEQFTLEHKGDQVYELKGNNGKYISSNMWCNSNGGSNSDQFLFTWGLKSA

SEQ ID NO: 102 - protein, 130 aa, synthetic construct
HVWTITLKGSNNKYVSSNNGSSAMMCDRNSVQGWEKFDVIDVGDGKVALRGTNGKYVSSNNGSSPMMCDRTSIGDWEKFTLVDLGNYQFAIKGSNGKYVSSEGGGSAMMCNRSNIGGWEVFTWGETTKSA

SEQ ID NO: 103 - protein, 131 aa, synthetic construct
GTSNQKVSLKNMNKYVSSEDGLKAVNCNRTSVGDWEKFEWITTSDNKTAFKGSNGKYLSSEDGLQSMTCSRSTIGDWEKFTVEHLGNQVYAIKGNNGLYVSSENGTKAMMCNRSTAGDWEKFAVTWGLKNA

SEQ ID NO: 104 - protein, 131 aa, synthetic construct
ATTLTPGSIVSLKGTNGKFVSSEDGKKTMNCNRSAVGAWEKFTVVDAGNGQVALKGSNNQYVSSENGVSFMNCNRASLGAWEKFTAVDAGNGQIALKGTNNKYVSSENGTKSMNCNRASLGAWEKFTVGQH

SEQ ID NO: 105 - protein, 132 aa, synthetic construct
ADKKVFTISLMNQKKFVSSEDGLKEMKCNRKQIGVYELFELKLLDGGKYSLKGSNMKYVSSNNGDSAMLCDRENLGNWEKFSLIHQSNNQFALKGNNNRFVSSENGTKGMRCDRSNPGDWEIFLLYEIVPKQ

SEQ ID NO: 106 - protein, 131 aa, synthetic construct
GPPFGQAITLKSVNNGFAGIEKGTALLKCDRQTSETFTVVNAGNGKVALLNMGKYMSGENGIKAVTCNRLIYGEWEKFSWIVNTDGTISLQDNNGKFLSVSDKNGSKVMKFHNTDSNTSERFVVTKLSDTK

SEQ ID NO: 107 - protein, 129 aa, synthetic construct
NAPPIGQSITLKGDNGDYVSSENGQIPMTCNRTTASAWEHFTVVDAGNGKVALQGNNGDYVSSENGVNPITCNRVSYSTWEEFTWISNADGSISLEGNNNCFVSSENGTQSMTCNRTIAQGWEDFLVNQ

SEQ ID NO: 108 - protein, 127 aa, synthetic construct
GSIPYGQTVTLKGFNNDYVSSENGTQPMTCTRTTAQAWEQFTVVDAGGGKVALQSMGKYVSSENGVNPITCNRTTIGDWEKFDWVVNADGTVAFRGNNGKYISSENGTQAMTCNRTTASGWEAFHVQ

SEQ ID NO: 109 - protein, 127 aa, synthetic construct
APPIGQTITLKGSNNLYVSSENGTQAMTCNRTTAQGWEQFLVVDAGNGKVSLQSTGKYVSSENGVNPITCNRTTASGWEQFNWISNSDGTISLQGNNGLFVSSENGAQAMTCNRTTIDGWEKFRVNQ

SEQ ID NO: 110 - protein, 129 aa, synthetic construct
SNPSIVGQTVTLKGFNNQYVNSQNGTQAMTCTSATAQAWEQFTVVDAGNGKVALRSMNKYVSSENGTQAITCNRTTIGDWEKFDLVPTADGKMSFRGSNGKFISSENGTQPMTCTRATASGWEAFGINQ

SEQ ID NO: 111 - protein, 133 aa, synthetic construct
GAPVGQTIWLRGSNGQYASSENGAVPMNCNRNSVQGWEQFTVVSAGGSKIALRGSNGKYVSSMNGQSGMMCDRTTVQGWEAFDWVSNANGTISLRGNNGLYVSSENGQSPMICDRTTIQGWEQFTWGTGAGAR

SEQ ID NO: 112 - protein, 132 aa, synthetic construct
TAPIGSTIWLRGNNNSYVSSENGTVPMMCNRTSVQGWEQFTVVDAGGGKIALRSMNKYVSSENGTASITCNRTTIGDWEKFDWIPTADGKVGLRGNNGRYISSENGVNPMTCTRTAISGWEAFSFGTGAGAR

SEQ ID NO: 113 - protein, 133 aa, synthetic construct
NPPINSVIWLRGPSNQYVTSANGAGPMYCNRPTVQAWEQFTVVSAGNGKVALRGSNGRYVSSENGVAAMTCNRTTIGGWEQFDWVDAGSGKIALRGSNGQYVSSENGAAAMICNRASIQAWEQFSWGTGVAAR

SEQ ID NO: 114 - protein, 135 aa, synthetic construct
PTNAPIGQTITLLGNNGRYVNGMNGTAAMQCNSAAVGTWEKFVVVDAGNGKVALRSMNKYVSSENGAATGITCNRAAIGGWEVFDWIANSNGTISLRGNNGFYVSSENGTKPMTCTRATSGGWEMFTYAITSAAR

SEQ ID NO: 115 - protein, 134 aa, synthetic construct
NNPAPIGQVITLKGNNGFYVSGENGAKAMTCTRTAPQAWEQFTVLDAGNGKVNLRSMGQYVSSENGTKAITCNRPAPQGWEAFDWIVNTDGSISLRGNNGFFVSSENGEEAMTCNRPTASGWEKFNFAIVSGAR

SEQ ID NO: 116 - protein, 134 aa, synthetic construct
NARISVGEVVSFKGSNGLFVCGENGTAPMQCTRTTASDWEKFTVVDAGGGKVALRSMGKYVSSENGTKAITCSRTAVGDWEKFTVVDNSDGSVSFLGNNGRYISSENGTKAMTCTRTTIGGWEKFTTGTSTPPT

SEQ ID NO: 117 - protein, 134 aa, synthetic construct
VTTAPIGKTIWLQGNNGLYVSSENGTQAMNCNRTTPQGWEQFLVGDAGNGKVTLQSMGKYVSSENGTQAITCNRATPQGWEQFDWVVNADNTISLRGSNGLYVSSENGAQAMTCTRTTIQGWEKFNFGIISGAR

SEQ ID NO: 118 - protein, 134 aa, synthetic construct
GNTAPIGQTITLRGFNNLFVSSENGTAPMWCNRTTAQAWEQFTVVDAGGGKVALRSMSKYISSENGAAPITCNRTTIGDWEKFDWVLTADGKVGFRGNNGLFISSENGANAMTCTRTSLGGWEAFSFVSVSGAR

SEQ ID NO: 119 - protein, 134 aa, synthetic construct
NPVAPIGKVITLRGNNSQYVSGENGTQALRCNRPSPQTWEQFSVIDAGGGKVALRSMGKYVSSENGTQAITCTRTTIGDWEKFDWIINTGGTISLRGNNGLYISSENGVAAMTCNRTSIQGWEVFTYAVVGNAR

SEQ ID NO: 120 - protein, 129 aa, synthetic construct
PSSPPIGTVISLKGFNNLYVSGENGTQAMNCNRTTAGDWEHFTVLDAGGGKIYLRSMGKYVSSENGTQAITCNRTTPGDWEKFDWIVTTDGKITLRGNNGKFISSENGTQAMTCNRAAASGWEAFGLNQ

SEQ ID NO: 121 - protein, 129 aa, synthetic construct
PNTPPIGTVISLKGFNGKYVSGENGTQAMTCTRTTAGDWEHFTVLDAGSGKIYLRSMGKYVSSENGTQAITCNRTTPSDWEKFDWIVTTDGKITLRGNNGKFISSENGTQAMTCNRATASGWEAFGLNQ

SEQ ID NO: 122 - protein, 135 aa, synthetic construct
NTRAPIGKIITLKAVSNGKYVSGENGTQAMTCTRATAGTWEQFSVLDAGNGKVYLRAMNMYVSSENGTQAITANRSSAGWWEAFDWIVNADGTISLGGYNGRYISSENGSQAMTCTRMTISGWEAFSYTVVGTAR

SEQ ID NO: 123 - protein, 135 aa, synthetic construct
NCGPPIGSTIWIQGFHGEYVSSNDGLGPMTCDSPSVGPNNRFVLEDAGDGRIALRGNNNLYVTSNNGRQPMACNQSEVDAWEQFSWVELSDGRVALRADHGQYASSNDGQDAMQCDRDVALAWESFLMGIEPECP

SEQ ID NO: 124 - protein, 135 aa, synthetic construct
NCAPPIGSTISLQGFHGEYVNTNGGNGDMTCDSAQVGPDNLFVLEEAGDGRVTLRGNNGRYVSSNNGVEAMTCDRFDVDAWERFSWVELPDGRVALRADHGQYASSNNGQEAMRCDRDTAFEWESFGATVESPCP

SEQ ID NO: 125 - protein, 135 aa, synthetic construct
NCAPPLGSTIWLQGFHGEFVNTNGGVGGMTCDSAEVGPDNLFVLEDAGDGRVTLRGTNQRYVSSNNGEQAMTCDQTEVDAWQKFSWVELPDGRIALRADHGQYASSNNGQEAMQCDRDIALAWESFAAGVEPPCP

SEQ ID NO: 126 - protein, 135 aa, synthetic construct
SGGAPIGQTIWLRGNNNQYVSSENGQGGMNCNRASVGGWEQFTVVAVGSAQIALQGSNGLYVSSGNGQTAMTCDASSISGWQAFTWEDQGNNTVALKGFGDQYVSSENGQTNMMCDRTSIGGWEVFTWGTGSGVR

SEQ ID NO: 127 - protein, 135 aa, synthetic construct
SPELPVGQVVWLRGNNGKYVSSENGQSAMMCNRDSAGDWEVFTLVGTADGKVALRGSNNQYVSSENGESPMLCNRTSIGGWEAFEWLPQPDGTVALRGFNGMYVSSENGEDPMLCNRPSVGAWEKFEFGLVSSGT

SEQ ID NO: 128 - protein, 134 aa, synthetic construct
PQTAPIGQTIWLRGNNSQYVCGENGTQAMRCNRASIGGWEQFTVVDAGGGKIALRSQNKYVSSENGTAAITCSRTTIGDWEKFDWIINADGKISLRGNNGRYISSENGTAAMTCSRTAIGGWEAFNWGTGTAGR

SEQ ID NO: 129 - protein, 134 aa, synthetic construct
AGAPIGQTIWLRGFNNQYVSSENGQAPMNCNRASAGGWEQFVVVSAGNGKIALRGSNGAYVSSENGLAPITCSRTTIGAWESFDWIEVSGGQVAFRGNNGQYISSENGQGPMTCTRTSISGWEAFNWGVAGSAR

SEQ ID NO: 130 - protein, 133 aa, synthetic construct
TSAPIGQTIWLRGINNLYVSGENGTTAMNCNRPSPGTWEQFTVVDAGAGKIALRSMSKYVSSENGVAAITCSRTTIGDWEKFDWVVNPDGKVSFRGNNGRYISSENGAAAMTCSRTTISGWEAFSYGTGSGAR

SEQ ID NO: 131 - protein, 134 aa, synthetic construct
SSAAPIGQIITLRGINNMFVSGENGTQAMRCNRATAGDWEKFSVLDGGNGTIMLRSMAKYVSSENGAAAITCSRATPGTWERFVWVVNTDGTISLRGNNNLYVSSENGTTAMTCTRAAISGWEAFNWTSVGTVT

SEQ ID NO: 132 - protein, 134 aa, synthetic construct
SSPAPIGQVITLRGTNNMYVSGENGTQAMRCNRATVGDWERFSVLDGGNGTIMLRSMSKYVSSENGAAAITCNRATPGGWERFTWVVNADNTISLRGNNNMYVSSENGTQAMTCNRATISGWEVFNWTSVGTVT

SEQ ID NO: 133 - protein, 134 aa, synthetic construct
ASVPVSEATVSLKANNGGYLSWQSSSPYLLTCQATSETANAQFTLVDAGAGRTALRASNGKFIRYSASDNKLYADATTLGSNEQFNLNRLNRSVAIRGPNSLFLTQNANDPVLCSKSILAGWEYFMVTTLSATQ

SEQ ID NO: 134 - protein, 133 aa, synthetic construct
VPVENGLNIIYLKGNNNKYVTYTNPGNLLTSTKQTLSNDEQFVIVDESEGNSALKCSNDRYLSLNPSDNRLYCDASMIGYEQKFRLNNLSGALSIMGSNGLYVSSEDGLITGMTCSRSVPSAWEFFNWGITGT

SEQ ID NO: 135 - protein, 148 aa, synthetic construct
GTGGGTGQPVNLRAANGQYVVAEGGGWGAVNANRDAAGPWETFALVDLNGGSLRDGDPVAFRTLSGSYLQAVSGGGDTMQAIGGDMGPWETFTIVDLDHPGGAVQSWDAVALRSCNGYFVVAELGGGDVVNVNRTAIGAWETFRIVVQ

SEQ ID NO: 136 - protein, 140 aa, synthetic construct
GNPAAGNRQHLTNSTGKYLNQVTTNGACGGAGVKADAGTITACTGLTRIPLGGGKVALQAENGLYLSCDNGGGAGVSANERTVSTYATFTETLASGKVSYKSSGGRYLTVASWGTLDLKCSAYLASTTEKFTPFANKASS

SEQ ID NO: 137 - protein, 139 aa, synthetic construct
AASQVPFQFALLASNGKYVSPQGGGGGQILVNGPTVGPWEPFGVEDVGFGKVAIRAANGLYLSPQDGGGDGKEVLANGPAVGEWEALDLITVGRGQIAFRTIRGFFLTRESIEGGRLMANATRMNEWEVFTFTNLFIGR

SEQ ID NO: 138 - protein, 141 aa, synthetic construct
KGLCHARHELVLKARVNGQFVCAEGGGDKNLVANRTSPGPWERFTVEQTNQEGRIHLRSSNGKYVCADLSGGNGQPVGLLVANRDSPGPWETFDLVEVDGGVIALKASNGKYVQASNGGSGPLIAGGDDHGWWEQFDVVEE

SEQ ID NO: 139 - protein, 138 aa, synthetic construct
STTGVATVISFQSRTNGLYVTAESAGSQPLIANRTAIGSWEQFDLVDAGNGAIALRSHADGMYVTAENAGAAPLIANRTAVGPWETFDLIVQPSTNGAVALRAHANGKYVTATNAGQGQLIATATVVGGWEQFDMILA

SEQ ID NO: 140 - protein, 138 aa, synthetic construct
STVGLATVVSLRSRTNNLYVTAENAGAQALIANRQAVGQWEQFDLVNAGNGAVALRSHANGLYVTADNAGAAPLIANRSAVGPWETFDLIVQPFTGAAVALRAHANGKYVSATNSGQGQLIATATQVGGWEQFDLLVS

SEQ ID NO: 141 - protein, 137 aa, synthetic construct
GPPPSTTTTVSLRARVNSRYVAAENAGASALIANRTAIGPWETFDLITNQDASISLRAHANNAYVTAENAGAAALIANRTAIGPWEKFDLIRNADGSVSLRAHANNAYVTAENAGAAALIANRTAIGTWEKFDLVSG

SEQ ID NO: 142 - protein, 138 aa, synthetic construct
GTNPQPTLVVALRSRASGFFVTAESAGASPLIANRSAVGGWERFQLLDAGNGRVALRSLANNRLVCAESSGSAPLVANRDAIGPWETFQLMRNADGSVALLSMANQRYVTTDAAGASPLLASRTTAGLTESFDLTPVA

SEQ ID NO: 143 - protein, 134 aa, synthetic construct
TPPSTAISLRSRANNMYVTAENAGAQPLIANRTAVGGWEQFDRIDLGNGNIALQARANGKYVCADNAGASALIANRDTIGGWETFQLVSNSDGTISLKAQANGKYVCADNAGAAALIANRDAVGPWESFDLASV

SEQ ID NO: 144 - protein, 134 aa, synthetic construct
NPSTTVISLRSRANNLYVCADNAGASPLIANRTTIGAWEQFDLLDAGGGNVALRAHANGQIVCADNAGVSPLIANRTAVGGWETFQLLRNADGTVSLRALADNNYVTAENAGAQALIANRASIGAWEKFDLIGA

SEQ ID NO: 145 - protein, 136 aa, synthetic construct
RQNGYNIVALKAGANNKYVSAENYGNDALIARSDTVSTWERFEQIDLGNNRIALRSLINGKYVTADNTGASPLIANKTAIGTWETFELGTTADGKRTLKSLANNLYVTAENGGNGSLIASRSSVGGAWETFELVKQ

SEQ ID NO: 146 - protein, 137 aa, synthetic construct
YNPPVGQTMTLKSSANNLFVSAGTDGNADLIANRSTAGTSEFFQVIDAGNGSVALKSLANGKYVSAATGTLKANKEAYTDDERFLWVDAGPGKIGLKAVAAGRFVCAENAGASALVANRTVVSGWESFIPAAVGPVD

SEQ ID NO: 147 - protein, 136 aa, synthetic construct
YASSGDIKIVGIKAYNGKYLGTEKSGKVEADRRKVERWETFELISLGNNKIAIRGYNGKYLCAEGGGGKDVVFNRDQIREWETFQLVYVSHGKYALKTYKGKYVCAENGGGGKVVANRDKVGKWETFEFVKSEFEL

SEQ ID NO: 148 - protein, 138 aa, synthetic construct
VSALAQNRRVALQAANGQYVAAINSGGGEIAARGPAISVWETLTMSVQPNNQVALQAPTGHYFCAENAGQQALVANRTAQGAWETFTRVDLGNDRIALRAVNGKYVCAEYGGGSYLIANRSAIGPWETFRLIDLGLIP

SEQ ID NO: 149 - protein, 138 aa, synthetic construct
SSNLKSLSAIQLQASTGHYFCAESGGGKEVIANRTAIGPWETFEFVDLGNGKGALKASNGQFVCAESGGGKGVTADRNAIGSWETFGFVELGNGKVALKASNGQFVCAESGGGKGVTADRNAIGPWETLKVITNRRIW

SEQ ID NO: 150 - protein, 138 aa, synthetic construct
MAETTETELIALRVHNGQYLCAEGGGGQSVVADRDKIDIWETFTLHQLGDSKVAIQAHNGQYVCAEGGGGQAVVANRDQIGPWETFTLHQLGDGKVAIQAHNGQYVCAEGGGGQSVVANRDQIGPWENFIYQTLAQSV

SEQ ID NO: 151 - protein, 138 aa, synthetic construct
ANFTPGCRSVALRANNGQYVCAVGGGGGEVVANRDQIKEWETFELIQLEGGKVALRANNGQYLSAAGGGGGKVLANQLHIKDWEIFEMIGAGPNQVALRTHNGKYVCAEGGGGRELAANRSTIGPWETFTLVEMEKAG

SEQ ID NO: 152 - protein, 138 aa, synthetic construct
EPVVKPDRKIALQAHNGQYVCVENGEGRLLVANRSQINDLETFKLIDLGDGKIALQAHNGQYISAENGGGRLLAANSRSIDHWETFKLIDLGDGKIALKAHNGHYVCAENGGGSCLVANRSNIDHWETFKLSIAVLSW

SEQ ID NO: 153 - protein, 132 aa, synthetic construct
MSKISLQASNGQYVCAEGGGGQQLVANRGTVGSWETFTLTDLGNGKIALQASNGQYVCAESGGGQQLVANRGAIGSWETFTLTDLGNGKIALQASNGQYVCAEGGGGQQLVANRSAIGPWETFIKNHLEETQ

SEQ ID NO: 154 - protein, 130 aa, synthetic construct
SGGTIDHQYVTLESHHHKYLSAQQNGTLQWNRDKIGEWEIFYVTKVANGYNIRSHHGKYLCVEQNGNVVCNRDNPAQWETFELKNKGDGISIKSWQGKFLSAQPNGSVEANRDKANEWEIFDVRLAPGAQ

SEQ ID NO: 155 - protein, 136 aa, synthetic construct
SDNGMLQGEISLKSVHGKYLNATPEGRAQWDDDVAHVGEYFRVEQRQNGKIALMSAFDMYVSAQPDGSVEINRREAPPGGWEEFTVEYRSNDVVCLKSCHGKYLSAQQDGTAQWNRDHAPIGGWEEIQIEQYRETS

SEQ ID NO: 156 - protein, 134 aa, synthetic construct
SESGMLPGEISLKGVHGKYLSATPEGRVLWNTDVAHVGEYFRVEQRQNGKIALRSAFDMYVSAQPDGRVECNRREAPPGGWEEFTVEYRSNDVVCLKSCHGKYLSAQKDGPWDWTMRQEADAQDIQIEPYRKTG

SEQ ID NO: 157 - protein, 130 aa, synthetic construct
MAQIALRSVHGKFLSAQPDGSAQWNRDVAGAWEYFHIEERQGGKITLRSAHGKYVSAQPDGSVQINREAAPPGGWEEFTAELRTNNVVCLKSCHGKYLSAQQNGTAQWNRDHAPSGGWEDIQIVHQGSTS

SEQ ID NO: 158 - protein, 136 aa, synthetic construct
SPKHMHEGKIALKSVHGKFLSAQPDGRAEWNRGIASDWEYFHLERREDGKITLRSTHGMYVSAQPDGTVQINRQAAPPTGWEEFTVENRGNNIVCLMSCHGKYLSAQLDGTAQWNRDHAPKGGWEDIQFIQQGGTG

SEQ ID NO: 159 - protein, 136 aa, synthetic construct
CLGIVMQGTVSLRSVHGKFLSAQPDGRAEWNRDVASSWEYFHIEERPGGKITLRGAHGMYVSAQPDGSVQINRQSAPPNGWEEFIVEDRGNNVICLKSVHSKYLSAQPDGTAQWNRDHAPRGGWEDIRFVPKGGTG

SEQ ID NO: 160 - protein, 132 aa, synthetic construct
MHEGKLALKSIHGKFLSAQPDGRAEWNRDLASDWEYFHLERRKDGKITLRSTHGMYVSAQPDGTVQINRKAAPHSGWEEFTVEDRGNDVVCLKSCHGKYLSAQPDGTAQWNRDHAPRGGWEDIQFVHQGGTG

SEQ ID NO: 161 - protein, 122 aa, synthetic construct
SSSGMLQGEIALKSVHGKYLNATPEGRAEWDDDIAHVGEYFRVEQLQNGKIALKGAFDMYVSAQLDGSVEINRREALEWEEFTVEYRRNDVVCLKSCHGKYLSAQPDGTAEWNRDHAPRGGW

SEQ ID NO: 162 - protein, 136 aa, synthetic construct
SSIEIDEGKIALKSVHGKYLSAQPDGRAEWNRDIASEWEYFHLEKRQGGKITLKGAHGMYVSAQTNGTVQINRQAAPPTGWEEFTVEDRGNNVICLKSVHWKYLSAQPDGRAEWNRDHAPPGGWEEFQIVHLGGIG

SEQ ID NO: 163 - protein, 144 aa, synthetic construct
SYDSLVEGKVAFKSVHGRYLSAQPDGRAEWNRDRADAWEYFHLEKRGNGKITLKSAHGKYVSAQPDGSVQINRGTAPRGGWEEFTVQLHFIKPVSGSGYDVVRLKSVHGKYLSAQPDGRAEWNRDHTPEGGWEDIQMISSGVAT

SEQ ID NO: 164 - protein, 144 aa, synthetic construct
SYDSLVEGKVAFKSVHGRYLSAQPDGRAEWNRDRANAWEYFHLEKRGNGKITLKSAHGKYVSAQPDGSVQINRGTAPRGGWEEFTVQLHFIKPVSGSGYDVVRLKSVHGKYLSAQPDGRAEWNRDHTPEGGWEDIQMISSGVAT

SEQ ID NO: 165 - protein, 130 aa, synthetic construct
MSSKVSLRSVHGKYLSAQPDGRAEWNRDIASTWEIFHLEERQGGKIALRGAHGMYVSSQSDGSVQINRREAPPGGWEEFTAEIRPNAVCLKSCHGKYLSAQQDGTAQWNRDHAPRGGWEEFQIEHTGGTG

SEQ ID NO: 166 - protein, 131 aa, synthetic construct
MQGTVSLRSVHGKFLSAQPDGRAEWNRDHASTWEYFHLEKRQGGKVALKGAHGMYVSAQPDGSVQINRREAPPGGWEEFTVEDRGNDVVCLKSCHGKYLSAQQNGTAQWNRDHAPRGGWEEFQIEHSGDTG

SEQ ID NO: 167 - protein, 136 aa, synthetic construct
KPELHMPSKVSLRSVHKKYLSAQPDGRAEWNRDIANEWEYFHIEERPGGKITLKSSHGKYVSAQADGSVQINRGAAPPGGWEEFTVEDRGNNVVCLKSCHGKYLSAQQNGTAQWNRDHAPRGGWEEFQIESSGGTG

SEQ ID NO: 168 - protein, 136 aa, synthetic construct
LEHIHGGARVALRSVHGKYLSAQPDGRAEWNRDHASTWEHFYLEKRQGGKIALRGVHGMYLSAQSDGSVQINRGAAPPGGWEEFTVEDRGNNVVCLRSCHGKYLSAQQNGTAQWNRDHAPRGGWEDIQFMQQGATG

SEQ ID NO: 169 - protein, 131 aa, synthetic construct
MHGTVSLRSVHGKFLSAQPDGRAEWNRDEAHDWEHFHVEKRHNGKITLKGVHGMYLSAQPDGSVQINRREAPPGGWEEFTVEDRGNNVVCLKSCHGKYLSAQQDGTAQWNRDHAPRGGWEDIQFVQQGATG

SEQ ID NO: 170 - protein, 135 aa, synthetic construct
NPLLPIGSSVAIKAMNGKYLSSENGTKAMTANRNSIGIWEKFTVVNAGNGLIALKGNNGRYVSSENGLKPIQCNRTQLGLWEKFKVVSIGNNFIALKGNNGKYIKTKNSGNTLICSNYNISWREKFSIKILNTTV

SEQ ID NO: 171 - protein, 131 aa, synthetic construct
IACDIIAIKGNNGKYVTSQNTNVPITANQLTVGDSEKFEVINRSNGQIALRGNNGRYVSSENGQKAMTCNRTAIGAWELFEVIDRGTSKIALRGNNGRYVSSENGNKPLNCNRSAIGAWEIFDVEEQCGGG

SEQ ID NO: 172 - protein, 143 aa, synthetic construct
AAKITNGQTISLKANNNKYVSSENGTKDMIANRSAIGSYEKFTVVKVGTKNGNDLIALKANNNKYVCSEDGTKDMIANRTAIGAWEKFEVIQLGTASGNELIALRASNNKYVCSENGTKDMRANRTAIGAYEKFQVINNGGST

SEQ ID NO: 173 - protein, 130 aa, synthetic construct
FDVTPSVVLYSFQNQGSFNYISSANPDVLNCNATSAGSTELFEIIDNGDGTVAFKGSNGLYVSSENGTKELTCTRTTIGAWEKFVMEDHGNDVYALKGNNDQYVRNNMLCTSPGASDWQKFKINGASSAR

SEQ ID NO: 174 - protein, 150 aa, synthetic construct
MDITLKADHGKYLCAEEGGGEQGQIIAGRQAGLVTATRDAAGAWETFVLVDADDDSVAFRSHGGKFLCAENEGRDGVLVCNRDAVGAWEKFIPHSDGDRMGFEAKCRPGFFIKAWPDGRVTLDQPMFNDAPATEPGPYELFETSVAIPGM

SEQ ID NO: 175 - protein, 141 aa, synthetic construct
GADTINETIIALKTDHDRYVSALSAQESPSYDLVADRTEVQGWEKFTLINTGDGKIALKTDHDRYVSAQESPSYDLVADRTEVQGWEKFTLINTGDGKIALKTDHDRYVSAQESPSYDLIADRIEVQGWEKFTVIDASNGT

SEQ ID NO: 176 - protein, 143 aa, synthetic construct
SNDIPYGKIIWLKANQGKAGYVVAERNLTNAPLKANRNRLGQWEKFRVENAGNGKVHLKALANNKYVQARINDNGKMQASTNNKLGWETFTWKSKGNGKVALKAFNNKYVQARLNLATKELAAIADTARDWETFNWGETTASG

SEQ ID NO: 177 - protein, 137 aa, synthetic construct
GGTIPIGSTISLQSRANNNYVTASATGASPLIASSATVGTAQLFTVVDMGNGCVALRAQINNDYVCADNGGAANLINNRTSAGLWETFQWIENTDGTISLKSNSDQEFVCAENAGANPLIANRPGHGLWEGFNYAIH

SEQ ID NO: 178 - protein, 132 aa, synthetic construct
VTNPNPATSLRSRANNRYVTAGSSALIADATSVGTAQKFDLVDLGNGNVALKARANNQYVCAENAGAASLIANRAAIGSWETFGLVHNSNGTVSFRAQANGRYVVAENAGAAALIANRTAIGPWEQFDLVTS

SEQ ID NO: 179 - protein, 136 aa, synthetic construct
RVVEPPAGASNLRARANNRYVSAANATTPLIANQAAIGATTRFDLVDLGGGNVALRFRAGNTYVCAEAAGASPLIANRGAAGGWETFALLRNSDGTVSLRSLANNRYVVAENGGAAALIANRTTVGPWEKFDLVTG

SEQ ID NO: 180 - protein, 142 aa, synthetic construct
ANMIKANDVIWLKSKISNQYVCADQNLGTEAPLFANRSTYGAWEKFEVKDAGDGWFRLWSVGTSKYVMSNQDNSSKLQANASAGSWEKMKFYYTSDGYVGMKYQINGKFVAAENYGTEQLIADRTSVGDWEKFSWGLTTKSA

SEQ ID NO: 181 - protein, 133 aa, synthetic construct
VNPPEANTALKAKVNGRYVSADGAGAKPLIASAPTASLWERFDVVELGNGQVGLLARVNNKFVSADGAGSKPLIANAPTASTWERFDIIRSSDGTISLRAAINGKFVSADGAGSKPLIANGPAISTWEKFTLG

SEQ ID NO: 182 - protein, 141 aa, synthetic construct
GRHRAPSTLIGLRSMANMLYVTAENAGNAPLIANRAALSGWETFTVVDLGNGLVALRAAANGLFVCADNQGKSPLIANRTTISTWESFQLVDNHDGTVSLRSQANNAFVCADSAGASPLIANRQAVGDWEKFMLISVTNTS

SEQ ID NO: 183 - protein, 135 aa, synthetic construct
TGTSSTPVVNLKSLTNNLYVSAPNATTALIANSSTVGVAQQFDLVDLGGGKVALRSKANNMYVCAENAGANPLLANRAAVGAWETFVLIRNSDGSESLQAQVNGMYVTAENNGAGSLIANRTAIGPWEKFDLVGA

SEQ ID NO: 184 - protein, 134 aa, synthetic construct
TGTGGGTGAITLRAQVNSQYVSAVGTNPLIADKATVTSTEQFDRVDAGNGAIALRSRANGMYVCAENAGAEPLVANRTTVGAWETFQLITNSDGTVSLRATVNNQIVAAENAGAGSLIANRTTVGAWERFTLTG

SEQ ID NO: 185 - protein, 135 aa, synthetic construct
TGGTGGGPATGLRASANNRYVSAVGSSPLIADKASIGATEQFDLVDLGGGNVALRSRNTGMYVCAENSGANPLVANRAAAGAWETFALVRNPDGTVSLRATVNNMYVAAENAGTEPLIANRTAIGPWEKFTLVNG

SEQ ID NO: 186 - protein, 137 aa, synthetic construct
GSEAPIGSIIAIKAMANNKYVTAGDAGNSALIANRDAAQGWEQFEVVDGGDGFIALKSLANGKYVTAENGGESNLIANRDSIQGWEQFKWIDNGDNTFSLQANANGLYVTAEDGGDSPLISNRPLIDGWEKFQYIIQ

SEQ ID NO: 187 - protein, 138 aa, synthetic construct
GTSAPIGSVISIKSMANQLFVTAEDGGDSALVANRDSVGTWEEFEVVDGGNGFIALKSLANNQFVSAEDEGESSLIANRSEVQGWEQFLWIDNGDGTFSLQANANGKYVTAEDSGDSPLIANRDSIGGWEKFQFTIQR

SEQ ID NO: 188 - protein, 141 aa, synthetic construct
GSGAPVGHTIWLQTTNNTNYVSARTDQSNTPLDANVTQVQAWEEFDVIDAGNGLIALRAHANNNYVSARKDQTNSPLEAIATQVQAWEEFRWLPQSNGTVALQAIANNNYVSARTDQTNTPLDANVTQVQGWEQFTWGQVS

SEQ ID NO: 189 - protein, 140 aa, synthetic construct
GSGAPVGKTIWLRATNNNNYVSTRIDQTNSPLNASATQVQAWEEFDVIDAGNGQIALLAHANNDYVSARTDQSNTPLDAMVNQLQGWEKFTWLPQSDGTVALQSQANGNYVSARTDQSNTPLDANVTQIQGWEKFTWGQV

SEQ ID NO: 190 - protein, 139 aa, synthetic construct
GSGAPIGKTIWLNATNGNYVSARIDQSNNPLNASATQVQAWEEFDVIDAGNGRIALKSHANGDYVSTRIDQSNNPLDASATQVQAWEEFTWLPQSDGTIALQAQANGNYVSARTDQNNTPLDAMVTQIQGWEKFTWGQV

SEQ ID NO: 191 - protein, 139 aa, synthetic construct
GSGAPIGKTIWLNATNGNYVSARIDQSNNPLNASATQVQAWEEFDVIDAGNGLIALKSHANGDYVSTRIDQSNNPLDASATQVQAWEEFTWLPQSDGTIALQAQANGDYVSARTDQNNTPLEAMVTQIQGWEKFTWGQV

SEQ ID NO: 192 - protein, 147 aa, synthetic construct
FAAAPIGQIISLKASATNLYVSADQDLDATAPLVADRTTVNTWEQFQVVDAGGGLVALLSIGNGKYVSADQNLGANTPLVASRTVFNTWEEFHWIDLGNGQIHLLNVGNGKYVSADLNIASTAPLNADRTTAQAWETFAWATVGGGG

SEQ ID NO: 193 - protein, 138 aa, synthetic construct
LFTQPNLPVISLRAHANGDYVTADNGGGSPLIANRTAIGFWEQFDEIDQGNGAIALRSHANGLIVTADNAGAQPLIANRTAVGAWETFQLIHNPDGSVSLKADANGDYVTAENAGAQPLIANRTAIGAWEEFDLIRDN

SEQ ID NO: 194 - protein, 138 aa, synthetic construct
LFTPPNLPVISLRAHANGDYVTAENGGGSALIANRTAIGYWEQFDEIDEGSGAIALRAHANGLLVTAEAAGAQPLIANRTVVGAWETFQLIHNADGSVSLKADANGDYVTAEAAGAQSLIANRTAIGGWEECDLIVDN

SEQ ID NO: 195 - protein, 143 aa, synthetic construct
GSAAPIGSTIALKANGNGKYLCADQYLDAVNWPVAANRNGIGAWEKFTVVDAGGGMVAFKSVGNGKYLCADKGLGDKLAANRTAIGAWEKFTWVQQSDGTVAIKANSNGKYLSSDKYLNATNWPVAANRAAVGAWEKFSSQQQ

SEQ ID NO: 196 - protein, 146 aa, synthetic construct
ADTGLIGKTVAFKALANNQYICADRYLNATHWYLAANRTSVGAWEKFLVVDAGSGLVAFKASGNGKYVCADQQLDAINWKLAANRTSVGAWEKFTIINNSDGTVSLKANGNGKYICADKGVNTNVPPLLSNRTAIGAWEKFTVEVQ

SEQ ID NO: 197 - protein, 132 aa, synthetic construct
MGNGKYVAADRSLSVVNWPLAANRTAVGAWEKFQVKDAGAGAISLTSVGNGKTVASDQRLSATNWPLAANRTAVGAWERFTWIENSDGTVSLKAHGNGQYIAADRFLDATNWPLAANRSAIGAWEKFTVEIQ

SEQ ID NO: 198 - protein, 134 aa, synthetic construct
NGDMLDGTQVQFKSVTLQKYVSAENGGGMNVTVDRDAPSSWETFTLWRLSELEFQFRTSQGQFFTCDGDRCSVSATAKSASTSESFYIERKEDNKIHIKTKNGAYLQATTYGQLTADYPGVPGWDDNAATFELT

SEQ ID NO: 199 - protein, 132 aa, synthetic construct
MKDGTEVQLKSVTLQTYVSAENGGGMNVAVDRDIPSSWETFRLWRVSETEFQFRTSKGQFLTCDGNGGSVSATAESPSRTETFYVEQNNKGRIHLKLMSGIYLQATSIANQLTADYPGTPGWDDNAATFEMT

SEQ ID NO: 200 - protein, 134 aa, synthetic construct
NGDMLDGTEVQLKSVALNKYVSAENGGGATIAVDRDIPQSWETFRLWRVSESIFQFRTSEGQFLTCDGDGATVTATAESASSNETFYLERNNDNRVHIKLKTGTYLQASKANQLTADYPGTPGWDDNAATFELI

SEQ ID NO: 201 - protein, 133 aa, synthetic construct
NGDMLDGTYVQLKSVTLQKYVSAENGGGMNVTVDRDIPSSWETFTLWRVSESEFQFRTSQGQFLTCDGDGGSVSSTAESPSRTETFYVERNNNRVHLKLNSGTYLQASMANQLTADYPSTPGWDDNAATFELM

SEQ ID NO: 202 - protein, 137 aa, synthetic construct
KMYPQTYDVITIKGNNNRFLSSNEGVPNTNGPKFDRTSARTLEHFRIIGLPGGKVALKGFNGKYLTVKPDNKVLCDGTYIGGLQQFKLEFRNKGVAIKGPNNKYVSSNNGDSRGVTCDRTRLGSYEFFQLSYKKKNV

SEQ ID NO: 203 - protein, 146 aa, synthetic construct
TSSAPIGKEIWIKSAQNKRYVSAKIRRTVPVLEADKDALGSWEKFNIVDARNGYIAIQSSANDNFISADKKISEDRPLVANRENISGSWEKFKWISLSGNKFALQSQANDKYIQAKIDQIGNPLLAKADAIKGWETFEWGIVNSLK

SEQ ID NO: 204 - protein, 145 aa, synthetic construct
QPIIPVGSIVWIKSNQNQKYITVESNLKKNFLRADKGSADIREQFLVIDAGNGYVALQSKANDFFVSADRKLSNDIPLVADREEIGNWEKFEWISISDNQFALKAKVNDKYVQARISSQGAPLMARGSVIKGWETFVYETVSQKN

SEQ ID NO: 205 - protein, 137 aa, synthetic construct
GGNGGTGGTVTIKAQSINKYISSENGSRSMRANRNSAGTQERFTVRSEGNGMVSFKGSNGKFVSHENGNKAMNCNRNVAQGWEKFTLVPQGGNVYAIKGSNGRYVSHENGSNNGITCNRTAIGSWEKFVIDGLTSNR

SEQ ID NO: 206 - protein, 146 aa, synthetic construct
FAAAPLNQTIWLRAVSSGKYVSADSNRGANTPLVADRDAAGGWEQFQVVDAGNGNIGLRAVANGKFVSADQNFANTPLVADRATMSGWEVFQWVDVTAGQVQLRSVGNNNFVSSDLNLGTHAPLVANRPTASGWETFNWGVVGANP

SEQ ID NO: 207 - protein, 146 aa, synthetic construct
FAAAPLGQTIWLRAISSGKYVSADANRGANSPLVADRDTANGWEQFQVVDAGNGYVGLRALATGKFVSADQNFANTPLVADRNTISGWEQFQWVDVTAGQVQLRSIGNNNFVSSDLNLGTHAPLVANRPTASGWETFNWGVVGTNP

SEQ ID NO: 208 - protein, 144 aa, synthetic construct
LSAREAHAATLRAVANNNYVSATATGTSFLTATAPVASTWEQFQVVNNANGTISLKATISGNYVAADTGLSAPDTSKLIANRGSIAGWEQFTVVPQSNGTVALRSMANGLYVSADLNIGGALVANRSSVAAWEQFVISDLPVTP

SEQ ID NO: 209 - protein, 144 aa, synthetic construct
SLPVDADAATLRAVANNNFVSATATGTSYLTATAGAAQSWEQFDVVNNGNGTVSFRARISGNFVSADLNLAQPNTGRLIANRRVADAWEQFTVVPQSNGTVALRSAANGQYVSADLNLGGVLVANRNAVQAWEQFVIADQPTTP

SEQ ID NO: 210 - protein, 143 aa, synthetic construct
AWNTAQAATIRAVANNAFVSATATGTSFLTATAQTASTWEQFEIVSNANGTVSFKATISGNYVSADIGLAAPNTSKLISNRTAINGWEQFTLVPQGNGTVAIRAAANNLYVSADLNIGGALVANRNTVQAWEQFVISDQPTTP

SEQ ID NO: 211 - protein, 143 aa, synthetic construct
AFGAAADAATLRAVANNNFVSATATGTSFLNATAPVASTWEDFEVIRNGNGTVSFRATISGNIVSADQGLGAPNTNRLIGNRQAINAWEQFTLETQSNGTVAIRSATTGLYVSADLNRGGFLVADRPTVQAWEQFVLSDTTTP

SEQ ID NO: 212 - protein, 135 aa, synthetic construct
PDTLHAAERIALQASDGTYIRAGVTKNTLLATGSPHIKGWETFTLIRLSDGKIALRSEQNGKYVRAGVGQNSLLAAVSSRIGGWEQFEFVGSGNTVALKSVQSGKCVRATEQGYLSATARECIDAEPKLSIVLLP

SEQ ID NO: 213 - protein, 137 aa, synthetic construct
LEWKKVYTVAIKSLINGKFVCAEKNGSQPLIANKDSFGLWETFEICFIDTQAIALKSYGNGKFVTAVNTDTNLLIANKDQATVWETFTLVPSFGTFGFKSHGNSKLVTAEEAGNKPLTANRDVLDVWEMFSLVYVWP

SEQ ID NO: 214 - protein, 136 aa, synthetic construct
GAPVAAPPLALRSWTNGKYVSADGRGSLPLIANGPTIGTWEKFTLINNSDGSVSLRADINGKYVSADGRGTLPLIANGPAIGTWEKFTLINNSDGSVSLRANINGKYVTAAGKGTLPLIASAPTISTWEKFDRFAA

SEQ ID NO: 215 - protein, 134 aa, synthetic construct
SPARDAHGLRARSNGKFVSADGAGSKPLVNNGPAFGAWERFDIVDAGSGLVALRAKVNGKFVSADGAGSKPLIANGPAVSTWEKFQLINNADGTVSLRAIVNNKFVSAPNTGGTLIANASTASTWEKFFVEAPN

SEQ ID NO: 216 - protein, 138 aa, synthetic construct
PALPGTIALRAHANGRYVTADSAGTAPLIADQGTVGQWERFDTIDAGNGYVGLRARANGRYVTAESAGAAPLIANRTGVAAWERFKLVDNADGSVSLLAAANGRYVTAQNAGAAPLIANRTAVSSWEKFDVVDPATLP

SEQ ID NO: 217 - protein, 133 aa, synthetic construct
AGPATSLLATINNRYVSAANAGAAPLIANRPALGVWEQFDQIDAGGGMVALRARINGLYVTAENAGAAPLVANRTAISTWEKFTVVTNADGTVSLRANANNRFVAAENAGRSALIANRTAIGPWEKFRAVKPP

SEQ ID NO: 218 - protein, 133 aa, synthetic construct
GGTPFGLRAHANGKYVTADNGGSSPLIANRTAVSAWERYDQVDAGGGYIALRSHANGKYVTAQNAGNGPLLAQATVIAAWEKFTVVVNPDASVSLRSFANGKYVTAENGGTSALIANRTAIGGWEQFDEVLPA

SEQ ID NO: 219 - protein, 135 aa, synthetic construct
GGIPSSISLRANVNNRYVVAENAGASSLIANRTGVGIWEQFFLVDVGGGYVALRSNANGRYVTAENAGGSALIANRGVVGEWEKFQRVANPDGSVSLRANANGRYVTAENAGSAPLIANRTAIGPWERFTQIGPS

SEQ ID NO: 220-222 - skipped (3 entries)

(19) United States
(12) Patent Application Publication
(10) Pub. No.: US 2025/0002721 A1
(43) Pub. Date: Jan. 2, 2025

(54) Novel Carbohydrate Binding Polypeptides

(71) Applicant: Glycolink AB, Malmö (SE)

(72) Inventors: Lauren McKee, Bromma (SE); Mengshu Hao, Sollentuna (SE); Qi Zhou, Täby (SE); Salla Koskela, Helsinki (FI)

(21) Appl. No.: 18/709,236

(22) PCT Filed: Nov. 15, 2022
(86) PCT No.: PCT/EP2022/081942

§ 371 Date: May 10, 2024

(30) Foreign Application Priority Data
Nov. 15, 2021 (EP) 21208133.5

(51) Int. Cl. C08L 89/00; C07K 14/195; C08B 37/00; C08L 5/00; C12N 15/70

(52) U.S. Cl. CPC C08L 89/00; C07K 14/195; C08B 37/006; C08L 5/00; C12N 15/70; C08L 2312/00; C12N 2800/101

(57) ABSTRACT

The present invention relates to novel carbohydrate binding polypeptides and their use in making hydrogels.

### TECHNICAL FIELD

The present invention relates to novel carbohydrate binding polypeptides and their use in making hydrogels.

### BACKGROUND

Hydrogels are a huge and booming business, with application in many fields including drug delivery, tissue scaffolding, life science research, the cosmetic industry, bio-based agriculture, and industrial lubrication. The majority of hydrogels are still made from fossil-derived polymers, built into a 3D network by chemical modification and cross-linking. This production method is unsustainable, and it is viewed unfavourably by consumers, so there is a pressing need to start using biological polymers for gel formation.

Hydrogels can already be produced from some natural polymers like cellulose and other polysaccharides of plant or microbial origin, as well as animal-derived mucins. But these still require chemical modification to permit cross-linking, leading to a high chemical waste footprint, and the potential inclusion of compounds that can irritate the skin or interfere with other biological systems, depending on the application.

There is a strong drive from industry to develop new methods of producing hydrogels from sustainably sourced polysaccharides such as cellulose and scleroglucan. Scleroglucan is secreted by industrial fungi such as Sclerotium rolfsii, and can be obtained with reasonably high yield and purity (Schmid et al. 2011). Prior to gel formation, in current practice scleroglucan is typically dissolved in DMSO in order to separate the triple helices of its structure (Palleschi et al. 2005). It is then oxidised using sodium periodate to generate scleraldehyde derivatives (Maeda et al. 2001) with in some cases further oxidation catalysed by sodium chlorite, which produces carboxylic groups, yielding a product termed sclerox (Crescenzi et al. 1983). This oxidised form can be cross-linked with 1,6-hexanedibromide to create a gel-forming network, and different sclerox:dihalide ratios give markedly different mechanical and rheological properties (Coviello et al. 1999; Coviello et al. 2001). More recently, an efficient alternative method was developed that uses boric acid (Borax) to directly cross-link the polysaccharide by means of 4,6-gluco-borate linkages and physical associations, creating a physical gel (Coviello et al. 2005; Palleschi et al. 2006). However, Borax is a regulated Substance of Very High Concern (SVHC) (European Chemicals Agency).

Thus, the need for improving sustainability of hydrogel production remains unsolved.

### SUMMARY

We here present an entirely new mode of polysaccharide cross-linking that requires no modification of the starting polymer and that uses no chemical cross-linking. The cross-linker is in fact a polypeptide belonging to a novel Carbohydrate Binding Module (CBM) family. Each polypeptide within the herein disclosed new CBM family, called CBM92, comprises three distinct repeat regions which are binding sites for mono-, oligo-, and polysaccharides. The polypeptides of this novel CBM family have binding specificity for polysaccharides with high molecular weight, e.g. scleroglucan, and gelation tendencies. The present inventors have demonstrated that proteins of this novel CBM family are highly advantageous as cross-linkers in hydrogel formation.

One aspect of the present disclosure relates to a polypeptide comprising at least two repeat regions selected from the group consisting of:
a. a repeat region 1 (R1), comprising or consisting of an amino acid sequence

| SEQ ID NO: 8: | | | | | | | | | | | | | | | | | | | | |
|---|---|---|---|---|---|---|---|---|---|---|---|---|---|---|---|---|---|---|---|---|
| I | W | L | - | Q | G | F | - | N | N | K | Y | V | N | S | K | N | G | - | - | - |
| 1 | 2 | 3 | 4 | 5 | 6 | 7 | 8 | 9 | 0 | 11 | 12 | 13 | 14 | 15 | 16 | 17 | 18 | 19 | 20 | 21 |
| Q | G | A | M | W | C | D | S | D | A | P | Q | A | W | E | L | F | | | | |
| 22 | 23 | 24 | 25 | 26 | 27 | 28 | 29 | 30 | 31 | 32 | 33 | 34 | 35 | 36 | 37 | 38 | | | | |

or a functional variant thereof wherein the amino acid sequence of said variant differs from SEQ ID NO:8 in that the amino acid sequence of the variant comprises 1 to 6 individual amino acid substitutions,
b. a repeat region 2 (R2), comprising or consisting of an amino acid sequence

| SEQ ID NO: 10: | | | | | | | | | | | | | | | | | | | | |
|---|---|---|---|---|---|---|---|---|---|---|---|---|---|---|---|---|---|---|---|---|
| I | A | L | R | G | N | - | N | G | M | Y | V | S | S | E | N | G | - | E | Q | A |
| 1 | 2 | 3 | 4 | 5 | 6 | 7 | 8 | 9 | 10 | 11 | 12 | 13 | 14 | 15 | 16 | 17 | 18 | 19 | 20 | 21 |
| I | T | C | N | R | P | A | I | Q | G | W | E | A | F | | | | | | | |
| 22 | 23 | 24 | 25 | 26 | 27 | 28 | 29 | 30 | 31 | 32 | 33 | 34 | 35 | | | | | | | |

or a functional variant thereof wherein the amino acid sequence of said variant differs from SEQ ID NO: 10 in that the amino acid sequence of the variant comprises 1 to 6 individual amino acid substitutions,
c. a repeat region 3 (R3), comprising or consisting of an amino acid sequence

| SEQ ID NO: 12: | | | | | | | | | | | | | | | | | | | | |
|---|---|---|---|---|---|---|---|---|---|---|---|---|---|---|---|---|---|---|---|---|
| V | S | L | R | G | - | S | N | G | L | F | I | S | S | E | N | G | A | - | - | - |
| 1 | 2 | 3 | 4 | 5 | 6 | 7N | 8 | 9 | 10 | 11 | 12 | 13 | 14 | 15 | 16 | 17 | 18 | 19 | 20 | 21 |
| A | A | M | T | C | T | R | - | P | T | A | - | S | G | W | E | A | F | | | |
| 22 | 23 | 24 | 25 | 26 | 27 | 28 | 29 | 30 | 31 | 32I | 33 | 34 | 35 | 36 | 37 | 38 | 39 | | | |

or a functional variant thereof wherein the amino acid sequence of said variant differs from SEQ ID NO: 12 in that the amino acid sequence of the variant comprises 1 to 6 individual amino acid substitutions.

Another aspect of the present disclosure relates to a polypeptide comprising at least two repeat regions selected from the group consisting of:
a. a repeat region 1 (R1), comprising or consisting of an amino acid sequence SEQ ID NO:220:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ X₃₁X₃₂X₃X₃₄WEX₃₇F,
wherein
X₁ is Isoleucine (I) or Valine (V);
X₂ is any amino acid, such as Tryptophan (W), Serine (S), Threonine (T) or Alanine (A);
X₃ is Leucine (L) or Isoleucine (I);
X₄ is Lysine (K), or Arginine (R), or is omitted;
X₅ is Glutamine (Q), Arginine (R), Alanine (A) or Lysine (K), or omitted;
X₆ is Glycine (G), Serine (S), Tyrosine (Y), Cysteine (C), Leucine (L) or Threonine (T);
X₇ is any amino acid, such as Phenylalanine (F) or Serine (S);
X₈ is Glycine (G), or is omitted;
X₉ is Asparagine (N), Histidine (H), Aspartic acid (D) or Threonine (T);
X₁₀ is Asparagine (N), Serine (S), Threonine (T), Glutamine (Q), Lysine (K) or Glycine (G);
X₁₁ is Lysine (K), Glutamine (Q), Asparagine (N) or Leucine (L);
X₁₂ is Tyrosine (Y), Leucine (L) or Phenylalanine (F);
X₁₃ is Valine (V), Leucine (L) or Alanine (A);
X₁₄ is Serine (S), Threonine (T), Cysteine (C) or Asparagine (N);
X₁₅ is Alanine (A), Serine (S) or Glycine (G);
X₁₆ is Glutamic acid (E), Lysine (K), Arginine (R), Glutamine (Q) or Aspartic acid (D);
X₁₇ is Asparagine (N), Glycine (G), Glutamine (Q) or Proline (P);
X₁₈ is Glycine (G), Asparagine (N), Threonine (T) or Aspartic acid (D);
X₁₉ is Leucine (L) or is omitted;
X₂₀ is Glycine (G) or Alanine (A), or is omitted;
X₂₁ is Alanine (A) or Asparagine (N), or is omitted;
X₂₂ is any amino acid, such as Threonine (T), or is omitted;
X₂₃ is any amino acid, such as Glycine (G);
X₂₄ is Proline (P), Alanine (A), Glutamine (Q) or Arginine (R);
X₂₅ is Leucine (L), Methionine (M), Alanine (A) or Isoleucine (I);
X₂₆ is any amino acid, such as Threonine (T);
X₂₇ is Alanine (A), Cysteine (C) or Tryptophan (W);
X₂₈ is Asparagine (N), Aspartic acid (D), Glutamic acid (E) or Arginine (R);
X₂₉ is Arginine (R), Alanine (A) or Serine (S);
X₃₀ is Threonine (T), Aspartic acid (D), Proline (P), Isoleucine (I), Asparagine (N) or Alanine (A);
X₃₁ is Threonine (T), Alanine (A), Valine (V), Glycine (G), Lysine (K) or Isoleucine (I);
X₃₂ is Alanine (A), Valine (V), Proline (P) or Leucine (L);
X₃₃ is Glycine (G), Glutamine (Q), Serine (S) or Aspartic acid (D);
X₃₄ is any amino acid, such as Glycine (G);
X₃₇ is any amino acid, such as Glutamine (Q) or Lysine (K);
or a functional variant thereof wherein the amino acid sequence of said variant differs from SEQ ID NO:220 in that the amino acid sequence of the variant comprises 1 to 6 individual amino acid substitutions,
b. a repeat region 2 (R2), comprising or consisting of an amino acid sequence SEQ ID NO:221:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁ WEX₃₄F,
wherein
X₁ is Isoleucine (I) or Valine (V);
X₂ is Alanine (A), Asparagine (N), Tyrosine (Y) or Threonine (T);
X₃ is Leucine (L) or Isoleucine (I);
X₄ is Glutamine (Q), Arginine (R), Leucine (L) or Lysine (K);
X₅ is Glycine (G), Alanine (A), Asparagine (N) or Serine (S);
X₆ is any amino acid, such as Alanine (A), or is omitted;
X₇ is Glycine (G) or Serine (S), or is omitted;
X₈ is Asparagine (N), Methionine (M), Glutamine (Q), Histidine (H) or Serine (S);
X₉ is Glycine (G) or Alanine (A);
X₁₀ is Lysine (K), Leucine (L), Methionine (M), Asparagine (N) or Serine (S);
X₁₁ is Tyrosine (Y);
X₁₂ is Valine (V) or Phenylalanine (F);
X₁₃ is Serine (S), Cysteine (C), Lysine (K) or Glutamine (Q);
X₁₄ is Alanine (A), Valine (V) or Serine (S);
X₁₅ is Glutamic acid (E), Aspartic acid (D), Glutamine (Q) or Glycine (G);
X₁₆ is Asparagine (N), Proline (P), Glycine (G), Threonine (T) or Aspartic acid (D);
X₁₇ is Glycine (G), Aspartic Acid (D), Isoleucine (I) or Asparagine (N), or is omitted;
X₁₈ is Valine (V) or Glycine (G), or is omitted;
X₁₉ is Glycine (G), Threonine (T), Leucine (L), Alanine (A), or Glutamic acid (E) or is omitted;
X₂₀ is any amino acid, such as Glutamine (Q), or omitted;
X₂₁ is Proline (P), Alanine (A), Glutamine (Q) or Asparagine (N);
X₂₂ is Leucine (L), Isoleucine (I), Methionine (M) or Valine (V);
X₂₃ is Threonine (T), Asparagine (N), Phenylalanine (F) or Arginine (R);
X₂₄ is Alanine (A), Cysteine (C) or Arginine (R);
X₂₅ is Asparagine (N), Alanine (A), Threonine (T) or Glutamine (Q);
X₂₆ is Alanine (A), Glycine (G) or Arginine (R);
X₂₇ is Threonine (T), Alanine (A), Lysine (K), Proline (P) or Leucine (L);
X₂₈ is any amino acid, such as Alanine (A);
X₂₉ is Isoleucine (I), Valine (V), Proline (P), Alanine (A), Lysine (K), Tyrosine (Y) or Leucine (L);
X₃₀ is Glycine (G), Alanine (A), Glutamic acid (E), Threonine (T), Serine (S), Glutamine (Q) or Aspartic acid (D);
X₃₁ is Glycine (G), Alanine (A), Aspartic Acid (D), Serine (S), Proline (P) or Lysine (K);
X₃₄ is any amino acid, such as Lysine (K);
or a functional variant thereof wherein the amino acid sequence of said variant differs from SEQ ID NO:221 in that the amino acid sequence of the variant comprises 1 to 6 individual amino acid substitutions,
c. a repeat region 3 (R3), comprising or consisting of an amino acid sequence SEQ ID NO:222:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁X₃₂X₃₃X₃₄X₃₅WEX₃₈F,
wherein
X₁ is Valine (V), Isoleucine (I), Tyrosine (Y) or Phenylalanine (F);
X₂ is Alanine (A), Serine (S), Cysteine (C), Threonine (T) or Glycine (G);
X₃ is Leucine (L), Phenylalanine (F) or Isoleucine (I);
X₄ is Arginine (R), Lysine (K) or Glutamine (Q),
X₅ is Glycine (G), Alanine (A) or Serine (S);
X₆ is Arginine (R) or Valine (V), or is omitted;
X₇ is any amino acid, such as Asparagine (N);
X₈ is Asparagine (N), Histidine (H), Alanine (A), Lysine (K), or omitted;
X₉ is Glycine (G), Tryptophan (W) or Asparagine (N);
X₁₀ is Lysine (K), Leucine (L), Methionine (M), Serine (S), Alanine (A) or Glutamine (Q);
X₁₁ is Tyrosine (Y), Tryptophan (W) or Phenylalanine (F);
X₁₂ is Valine (V), Leucine (L), Methionine (M) or Isoleucine (I);
X₁₃ is Serine (S) or Glutamine (Q);
X₁₄ is Alanine (A), Serine (S), Histidine (H) or Glycine (G);
X₁₅ is Glutamic acid (E), Aspartic acid (D), Glutamine (Q), Serine (S) or Asparagine (N);
X₁₆ is Asparagine (N), Leucine (L), Tryptophan (W), Methionine (M) or Aspartic acid (D);
X₁₇ is Glycine (G), Serine (S) or Aspartic Acid (D);
X₁₈ is any amino acid, such as Serine (S) or Threonine (T);
X₁₉ is Asparagine (N), Valine (V) or Aspartic acid (D), or is omitted;
X₂₀ is Alanine (A), or is omitted;
X₂₁ is Serine (S), or is omitted;
X₂₂ is any amino acid, such as Alanine (A), or is omitted;
X₂₃ is Proline (P), Alanine (A), Arginine (R), Threonine (T) or Glycine (G);
X₂₄ is Leucine (L), Methionine (M), Valine (V) or Isoleucine (I);
X₂₅ is Threonine (T), Tyrosine (Y), Asparagine (N), Leucine (L) or Glutamine (Q);
X₂₆ is Alanine (A), Cysteine (C), Phenylalanine (F) or Tryptophan (W);
X₂₇ is Asparagine (N), Alanine (A), Threonine (T) or Serine (S);
X₂₈ is Arginine (R) or Lysine (K);
X₂₉ is Aspartic acid (D), or is omitted;
X₃₀ is Threonine (T), Alanine (A), Proline (P), Serine (S) or Glycine (G);
X₃₁ is any amino acid, such as Alanine (A);
X₃₂ is Isoleucine (I), Glutamic acid (E), Proline (P), Alanine (A) or Valine (V);
X₃₃ is Glycine (G), Lysine (K) or Aspartic acid (D), or is omitted;
X₃₄ is Glycine (G), Serine (S), Lysine (K), Aspartic acid (D) or Glutamine (Q);
X₃₅ is Glycine (G), Alanine (A), Aspartic acid (D) or Cysteine (C);
X₃₈ is any amino acid, such as Lysine (K);
or a functional variant thereof wherein the amino acid sequence of said variant differs from SEQ ID NO:222 in that the amino acid sequence of the variant comprises 1 to 6 individual amino acid substitutions.

A further aspect of the present disclosure relates to an amino acid sequence having at least 75% sequence identity to SEQ ID NO:3 or SEQ ID NO:4, or any of the sequences SEQ ID NO:20 to SEQ ID NO:33, or any of the sequences SEQ ID NO:71 to SEQ ID NO: 219, and wherein said polypeptide is capable of binding one or more saccharide units.

A further aspect of the present disclosure relates to a polypeptide comprising an amino acid sequence having at least 75% sequence identity to SEQ ID NO:3, SEQ ID NO:4, SEQ ID NO:20 to SEQ ID NO:33, and wherein said polypeptide is capable of binding one or more saccharide units.

A further aspect of the present disclosure relates to a polynucleotide encoding a polypeptide disclosed herein.

Another aspect of the present disclosure relates to a recombinant vector comprising a polynucleotide disclosed herein, or a nucleic acid sequence encoding a carbohydrate-binding polypeptide disclosed herein.

Another aspect of the present disclosure relates to a host cell expressing the recombinant vector disclosed herein.

Another aspect of the present disclosure relates to a use of at least one polypeptide disclosed herein for cross-linking two or more polysaccharides.

A further aspect of the present disclosure relates to a hydrogel comprising at least one polypeptide disclosed herein and two or more polysaccharides, wherein said hydrogel has a cross-linked structure.

A further aspect of the present disclosure relates to a cross-linker for crosslinking polysaccharides, wherein said cross-linker is a polypeptide as disclosed herein.

Another aspect of the present disclosure relates to a method of manufacturing a hydrogel, the method comprising
a. providing a solution comprising a polysaccharide,
b. providing a solution comprising a polypeptide according to the present disclosure,
c. mixing the solutions under continuous stirring at room temperature,
thereby obtaining a hydrogel via cross-linking of the polysaccharide.

### DESCRIPTION OF DRAWINGS

FIG. 1: Repeating structural unit of scleroglucan polysaccharide.

FIG. 2: Cartoon visualisation of the superimposed crystal structures of Cp-F1 and Cp-F2. Glucose monosaccharide ligands are illustrated in two of the binding sites. Top arrow shows glucose in the alpha binding site, bottom arrow shows glucose in the beta binding site.

FIG. 3: Cartoon visualisation of the crystal structure of Cp-F1, showing a close up of Cp-F2 binding site bound to the ligand glucose, with the relevant amino acids indicated.

FIG. 4: Visualisation of the solid gel forming. Top: scleroglucan only. Bottom: scleroglucan and Cp-F1 protein mixed.

FIG. 5: Altering the ratio (9:1, 8:1 and 2:1) of polysaccharide to protein alters gel properties such as solidity and transparency. The hydrogels were made by mixing a stock solution of Scleroglucan having concentration of 5 g/L with a stock solution of protein (Cp-F1) having concentration of 4 g/L. Thus, the ratios are based on the volumes of the stock solutions mixed.

FIG. 6A-B: Example rheological assessment. All analyses shown relate to a hydrogel comprising Scleroglucan at a final concentration of 4 g/L. From low to high the final protein concentrations in the hydrogel are 0.08 g/L, 0.2 g/L, 0.4 g/L, 0.8 g/L, 1.2 g/L, 1.6 g/L and 2 g/L.

FIG. 7: Greyscale heat map of polysaccharide binding data for 12 proteins, and 5 variants of the Cp-F1 protein.

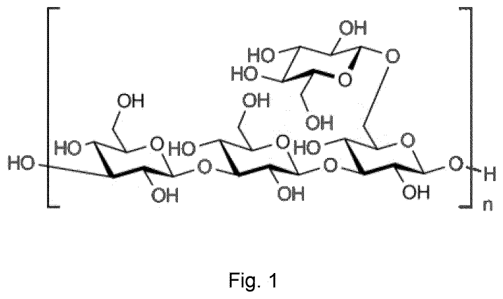

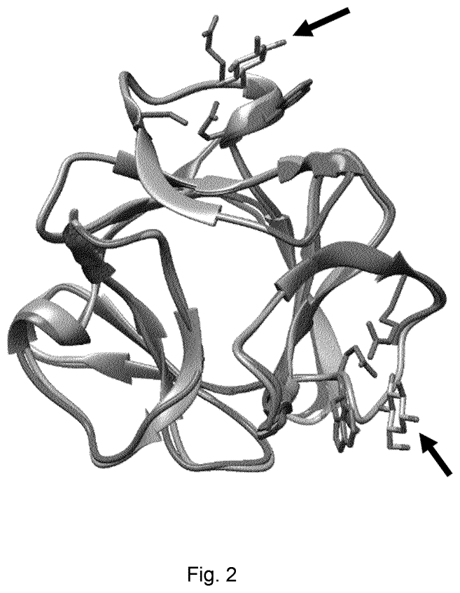

### DETAILED DESCRIPTION

### Definitions

As used herein, the term “polypeptide” refers to a single polypeptide chain which may or may not be modified by addition of non-amino acid groups. As the term does not refer to a specific length of the product, peptides, oligopeptides, and proteins are included within the definition of polypeptide. In an embodiment, the term “polypeptides” as used herein also include variants, mutants, modifications, analogous and/or derivatives of the polypeptides of the disclosure as described herein. Amino acid sequence mutants of the polypeptides of the present disclosure can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxyl terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxyl group present at the carboxyl terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822 (b)(2) belong to the group of amino acids listed herewith: Y, G, F, M, A, S, I, L, T, V, P, K, H, Q, E, W, R, D, N and C. Non-natural amino acids are those not listed immediately above. Also, non-natural amino acid residues include, but are not limited to, modified amino acid residues, L-amino acid residues, and stereoisomers of D-amino acid residues.

A “functional variant” of a peptide is a peptide capable of performing essentially the same functions as the peptide it is a functional variant of. In particular, a functional variant can bind the same molecules, preferably with the same affinity, as the peptide it is a functional variant of.

The terms “polynucleotide” and “nucleic acid” are used interchangeably herein, and can refer to any nucleic acid that contains the information necessary for the purpose indicated by the context. That is, the nucleic acid can be DNA or RNA, either single stranded or double stranded, or other nucleic acid, as long as it is capable of representing the appropriate information, e.g., in relation to an encoded peptide, and can include complementary sequences, e.g., sense strands and anti-sense strands of nucleic acids polymers.

As used herein, the term “binding site” refers to a region of a molecule or molecular complex that, as a result of its shape, favourably associates with another molecule, molecular complex, chemical entity or compound. The polypeptide of the disclosure described herein contains carbohydrate binding sites, meaning binding sites capable of associating with sugars, e.g. monosaccharides, disaccharides, and polysaccharides.

As used herein, the term “hydrogel” or “hydrophilic gel” refers to a continuous phase of a hydrophilic polymer that is capable of swelling on contact with water and other hydrophilic swelling agents. The term is used regardless of the state of hydration. A “hydrogel” refers to a material of solid or semi-solid texture that comprises water. Hydrogels are formed by a three-dimensional network of molecular structures within which water, among other substances, may be held. The three-dimensional molecular network may be held together by covalent chemical bonds, or by ionic bonds, or by any combination thereof.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide or oligosaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose). The term “saccharide” is used herein as a generic term for polymers of sugars and may refer to “monosaccharide”, “disaccharide”, “polysaccharide”, “carbohydrate”, or “oligosaccharide” and other synonyms.

### Carbohydrate-Binding Polypeptides

A carbohydrate binding module (CBM) is a low molecular weight, non-catalytic protein that can bind to a specific carbohydrate ligand. CBMs are classified by sequence homology into families on the CAZy database (www.cazy.org) (Lombard et al. 2014). The mode of CBM-ligand binding is determined by the surface architecture of the CBM protein structure (Boraston et al. 2004). In most CBM families, the proteins present one carbohydrate binding site per protein.

One aspect of the present disclosure relates to novel polypeptides which belong to members of the CBM family 92 (CBM92). In one aspect, the novel polypeptides of the present disclosure are capable of binding at least two polysaccharides, preferably three polysaccharides, and cross-link them. The obtained cross-linked structure comprising the novel polypeptides of the present disclosure and two or three polysaccharides, in presence of water, forms a hydrogel. Thus, in one aspect of the present disclosure, the novel polypeptides are characterized by comprising two or preferably three domains or regions which are capable of binding to certain polysaccharides.

In one aspect the present disclosure relates to a polypeptide comprising at least two repeat regions selected from the group consisting of:
a. a repeat region 1 (R1), comprising or consisting of an amino acid sequence

| SEQ ID NO: 8: | | | | | | | | | | | | | | | | | | | | |
|---|---|---|---|---|---|---|---|---|---|---|---|---|---|---|---|---|---|---|---|---|
| I | W | L | - | Q | G | F | - | N | N | K | Y | V | N | S | N | K | G | - | - | - |
| 1 | 2 | 3 | 4 | 5 | 6 | 7 | 8 | 9 | 10 | 11 | 12 | 13 | 14 | 15 | 16 | 17 | 18 | 19 | 20 | 21 |
| Q | G | A | M | W | C | D | S | D | A | P | Q | A | W | E | L | F | | | | |
| 22 | 23 | 24 | 25 | 26 | 27 | 28 | 29 | 30 | 31 | 32 | 33 | 34 | 35 | 36 | 37 | 38 | | | | |

or a functional variant thereof wherein the amino acid sequence of said variant differs from SEQ ID NO:8 in that the amino acid sequence of the variant comprises 1 to 6 individual amino acid substitutions,
b. a repeat region 2 (R2), comprising or consisting of an amino acid sequence

| SEQ ID NO: 10: | | | | | | | | | | | | | | | | | | | | |
|---|---|---|---|---|---|---|---|---|---|---|---|---|---|---|---|---|---|---|---|---|
| I | A | L | R | G | N | - | N | G | M | Y | V | S | S | E | N | G | - | E | Q | A |
| 1 | 2 | 3 | 4 | 5 | 6 | 7 | 8 | 9 | 10 | 11 | 12 | 13 | 14 | 15 | 16 | 17 | 18 | 19 | 20 | 21 |
| I | T | C | N | R | P | A | I | Q | G | W | E | A | F | | | | | | | |
| 22 | 23 | 24 | 25 | 26 | 27 | 28 | 29 | 30 | 31 | 32 | 33 | 34 | 35 | | | | | | | |

or a functional variant thereof wherein the amino acid sequence of said variant differs from SEQ ID NO: 10 in that the amino acid sequence of the variant comprises 1 to 6 individual amino acid substitutions,
c. a repeat region 3 (R3), comprising or consisting of an amino acid sequence

| SEQ ID NO: 12: | | | | | | | | | | | | | | | | | | | | |
|---|---|---|---|---|---|---|---|---|---|---|---|---|---|---|---|---|---|---|---|---|
| V | S | L | R | G | - | S | N | G | L | F | I | S | S | E | N | G | A | - | - | - |
| 1 | 2 | 3 | 4 | 5 | 6 | 7 | 8 | 9 | 10 | 11 | 12 | 13 | 14 | 15 | 16 | 17 | 18 | 19 | 20 | 21 |
| A | A | M | T | C | T | R | - | P | T | A | - | S | G | W | E | A | F | | | |
| 22 | 23 | 24 | 25 | 26 | 27 | 28 | 29 | 30 | 31 | 32 | 33 | 34 | 35 | 36 | 37 | 38 | 39 | | | |

or a functional variant thereof wherein the amino acid sequence of said variant differs from SEQ ID NO: 12 in that the amino acid sequence of the variant comprises 1 to 6 individual amino acid substitutions.

In one aspect the present disclosure relates to a polypeptide comprising at least two repeat regions selected from the group consisting of:
a. a repeat region 1, comprising or consisting of an amino acid sequence SEQ ID NO: 220:
X₁ X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁X₃₂X₃₃X₃₄WEX₃₇F,
wherein
   X₁ is Isoleucine (I) or Valine (V);
   X₂ is any amino acid, such as Tryptophan (W), Serine (S), Threonine (T) or Alanine (A);
   X₃ is Leucine (L) or Isoleucine (I);
   X₄ is Lysine (K), or Arginine (R), or is omitted;
   X₅ is Glutamine (Q), Arginine (R), Alanine (A) or Lysine (K), or omitted;
   X₆ is Glycine (G), Serine (S), Tyrosine (Y), Cysteine (C), Leucine (L) or Threonine (T);
   X₇ is any amino acid, such as Phenylalanine (F) or Serine (S);
   X₈ is Glycine (G), or is omitted;
   X₉ is Asparagine (N), Histidine (H), Aspartic acid (D) or Threonine (T);
   X₁₀ is Asparagine (N), Serine (S), Threonine (T), Glutamine (Q), Lysine (K) or Glycine (G);
   X₁₁ is Lysine (K), Glutamine (Q), Asparagine (N) or Leucine (L);
   X₁₂ is Tyrosine (Y), Leucine (L) or Phenylalanine (F);
   X₁₃ is Valine (V), Leucine (L) or Alanine (A);
   X₁₄ is Serine (S), Threonine (T), Cysteine (C) or Asparagine (N);
   X₁₅ is Alanine (A), Serine (S) or Glycine (G);
   X₁₆ is Glutamic acid (E), Lysine (K), Arginine (R), Glutamine (Q) or Aspartic acid (D);
   X₁₇ is Asparagine (N), Glycine (G), Glutamine (Q) or Proline (P);
   X₁₈ is Glycine (G), Asparagine (N), Threonine (T) or Aspartic acid (D);
   X₁₉ is Leucine (L) or is omitted;
   X₂₀ is Glycine (G) or Alanine (A), or is omitted;
   X₂₁ is Alanine (A) or Asparagine (N), or is omitted;
   X₂₂ is any amino acid, such as Threonine (T), or is omitted;
   X₂₃ is any amino acid, such as Glycine (G);
   X₂₄ is Proline (P), Alanine (A), Glutamine (Q) or Arginine (R);
   X₂₅ is Leucine (L), Methionine (M), Alanine (A) or Isoleucine (I);
   X₂₆ is any amino acid, such as Threonine (T);
   X₂₇ is Alanine (A), Cysteine (C) or Tryptophan (W);
   X₂₈ is Asparagine (N), Aspartic acid (D), Glutamic acid (E) or Arginine (R);
   X₂₉ is Arginine (R), Alanine (A) or Serine (S);
   X₃₀ is Threonine (T), Aspartic acid (D), Proline (P), Isoleucine (I), Asparagine (N) or Alanine (A);
   X₃₁ is Threonine (T), Alanine (A), Valine (V), Glycine (G), Lysine (K) or Isoleucine (I);
   X₃₂ is Alanine (A), Valine (V), Proline (P) or Leucine (L);
   X₃₃ is Glycine (G), Glutamine (Q), Serine (S) or Aspartic acid (D);
   X₃₄ is any amino acid, such as Glycine (G);
   X₃₇ is any amino acid, such as Glutamine (Q) or Lysine (K);
or a functional variant thereof wherein the amino acid sequence of said variant differs from SEQ ID NO:220 in that the amino acid sequence of the variant comprises 1 to 6 individual amino acid substitutions,
b. a repeat region 2, comprising or consisting of an amino acid sequence SEQ ID NO: 221:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁WEX₃₄F,
wherein
   X₁ is Isoleucine (I) or Valine (V);
   X₂ is Alanine (A), Asparagine (N), Tyrosine (Y) or Threonine (T);
   X₃ is Leucine (L) or Isoleucine (I);
   X₄ is Glutamine (Q), Arginine (R), Leucine (L) or Lysine (K);
   X₅ is Glycine (G), Alanine (A), Asparagine (N) or Serine (S);
   X₆ is any amino acid, such as Alanine (A), or is omitted;
   X₇ is Glycine (G) or Serine (S), or is omitted;
   X₈ is Asparagine (N), Methionine (M), Glutamine (Q), Histidine (H) or Serine (S);
   X₉ is Glycine (G) or Alanine (A);
   X₁₀ is Lysine (K), Leucine (L), Methionine (M), Asparagine (N) or Serine (S);
   X₁₁ is Tyrosine (Y);
   X₁₂ is Valine (V) or Phenylalanine (F);
   X₁₃ is Serine (S), Cysteine (C), Lysine (K) or Glutamine (Q);
   X₁₄ is Alanine (A), Valine (V) or Serine (S);
   X₁₅ is Glutamic acid (E), Aspartic acid (D), Glutamine (Q) or Glycine (G);
   X₁₆ is Asparagine (N), Proline (P), Glycine (G), Threonine (T) or Aspartic acid (D);
   X₁₇ is Glycine (G), Aspartic Acid (D), Isoleucine (I) or Asparagine (N), or is omitted;
   X₁₈ is Valine (V) or Glycine (G), or is omitted;
   X₁₉ is Glycine (G), Threonine (T), Leucine (L), Alanine (A), or Glutamic acid (E) or is omitted;
   X₂₀ is any amino acid, such as Glutamine (Q), or omitted;
   X₂₁ is Proline (P), Alanine (A), Glutamine (Q) or Asparagine (N);
   X₂₂ is Leucine (L), Isoleucine (I), Methionine (M) or Valine (V);
   X₂₃ is Threonine (T), Asparagine (N), Phenylalanine (F) or Arginine (R);
   X₂₄ is Alanine (A), Cysteine (C) or Arginine (R);
   X₂₅ is Asparagine (N), Alanine (A), Threonine (T) or Glutamine (Q);
   X₂₆ is Alanine (A), Glycine (G) or Arginine (R);
   X₂₇ is Threonine (T), Alanine (A), Lysine (K), Proline (P) or Leucine (L);
   X₂₈ is any amino acid, such as Alanine (A);
   X₂₉ is Isoleucine (I), Valine (V), Proline (P), Alanine (A), Lysine (K), Tyrosine (Y) or Leucine (L);
   X₃₀ is Glycine (G), Alanine (A), Glutamic acid (E), Threonine (T), Serine (S), Glutamine (Q) or Aspartic acid (D);
   X₃₁ is Glycine (G), Alanine (A), Aspartic Acid (D), Serine (S), Proline (P) or Lysine (K);
   X₃₄ is any amino acid, such as Lysine (K);
or a functional variant thereof wherein the amino acid sequence of said variant differs from SEQ ID NO:221 in that the amino acid sequence of the variant comprises 1 to 6 individual amino acid substitutions,
c. a repeat region 3, comprising or consisting of an amino acid sequence SEQ ID NO: 222:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁X₃₂X₃X₃₄X₃₅WEX₃₈F,
wherein
   X₁ is Valine (V), Isoleucine (I), Tyrosine (Y) or Phenylalanine (F);
   X₂ is Alanine (A), Serine (S), Cysteine (C), Threonine (T) or Glycine (G);
   X₃ is Leucine (L), Phenylalanine (F) or Isoleucine (I);
   X₄ is Arginine (R), Lysine (K) or Glutamine (Q),
   X₅ is Glycine (G), Alanine (A) or Serine (S);
   X₆ is Arginine (R) or Valine (V), or is omitted;
   X₇ is any amino acid, such as Asparagine (N);
   X₈ is Asparagine (N), Histidine (H), Alanine (A), Lysine (K), or omitted;
   X₉ is Glycine (G), Tryptophan (W) or Asparagine (N);
   X₁₀ is Lysine (K), Leucine (L), Methionine (M), Serine (S), Alanine (A) or Glutamine (Q);
   X₁₁ is Tyrosine (Y), Tryptophan (W) or Phenylalanine (F);
   X₁₂ is Valine (V), Leucine (L), Methionine (M) or Isoleucine (I);
   X₁₃ is Serine (S) or Glutamine (Q);
   X₁₄ is Alanine (A), Serine (S), Histidine (H) or Glycine (G);
   X₁₅ is Glutamic acid (E), Aspartic acid (D), Glutamine (Q), Serine (S) or Asparagine (N);
   X₁₆ is Asparagine (N), Leucine (L), Tryptophan (W), Methionine (M) or Aspartic acid (D);
   X₁₇ is Glycine (G), Serine (S) or Aspartic Acid (D);
   X₁₈ is any amino acid, such as Serine (S) or Threonine (T);
   X₁₉ is Asparagine (N), Valine (V) or Aspartic acid (D), or is omitted;
   X₂₀ is Alanine (A), or is omitted;
   X₂₁ is Serine (S), or is omitted;
   X₂₂ is any amino acid, such as Alanine (A), or is omitted;
   X₂₃ is Proline (P), Alanine (A), Arginine (R), Threonine (T) or Glycine (G);
   X₂₄ is Leucine (L), Methionine (M), Valine (V) or Isoleucine (I);
   X₂₅ is Threonine (T), Tyrosine (Y), Asparagine (N), Leucine (L) or Glutamine (Q);
   X₂₆ is Alanine (A), Cysteine (C), Phenylalanine (F) or Tryptophan (W);
   X₂₇ is Asparagine (N), Alanine (A), Threonine (T) or Serine (S);
   X₂₈ is Arginine (R) or Lysine (K);
   X₂₉ is Aspartic acid (D), or is omitted;
   X₃₀ is Threonine (T), Alanine (A), Proline (P), Serine (S) or Glycine (G);
   X₃₁ is any amino acid, such as Alanine (A);
   X₃₂ is Isoleucine (I), Glutamic acid (E), Proline (P), Alanine (A) or Valine (V);
   X₃₃ is Glycine (G), Lysine (K) or Aspartic acid (D), or is omitted;
   X₃₄ is Glycine (G), Serine (S), Lysine (K), Aspartic acid (D) or Glutamine (Q);
   X₃₅ is Glycine (G), Alanine (A), Aspartic acid (D) or Cysteine (C);
   X₃₈ is any amino acid, such as Lysine (K);
or a functional variant thereof wherein the amino acid sequence of said variant differs from SEQ ID NO:222 in that the amino acid sequence of the variant comprises 1 to 6 individual amino acid substitutions.

In one embodiment of the present disclosure, each repeat region of the polypeptide comprises a key Tryptophan (W) residue, preferably at position 35 of repeat region 1, position 32 of repeat region 2, and position 36 of repeat region 3, based on the amino acid numbering provided herein. Said Tryptophan (W) residue plays a key role in the binding between the repeat region that comprises it and a polysaccharide or disaccharide, as evident from the knock-out binding site mutants harbouring Tryptophan (W) to Alanine (A) substitution. The ring of the Tryptophan side chain has a major interaction with the carbon ring in a disaccharide or polysaccharide to which the polypeptide binds.

Thus, in one embodiment of the present disclosure, the polypeptide described herein comprises a repeat region 1, comprising an amino acid sequence SEQ ID NO:8, SEQ ID NO: 9, SEQ ID NO:34, SEQ ID NO:37, SEQ ID NO:40, SEQ ID NO:43, SEQ ID NO: 46, SEQ ID NO: 49, SEQ ID NO: 52, SEQ ID NO:55, SEQ ID NO:58, SEQ ID NO: 61, SEQ ID NO:64, SEQ ID NO:67 or SEQ ID NO:220, or a functional variant thereof wherein the amino acid sequence of said variant differs from SEQ ID NO:8, SEQ ID NO:9, SEQ ID NO:34, SEQ ID NO:37, SEQ ID NO:40, SEQ ID NO:43, SEQ ID NO: 46, SEQ ID NO: 49, SEQ ID NO: 52, SEQ ID NO:55, SEQ ID NO:58, SEQ ID NO: 61, SEQ ID NO:64, SEQ ID NO:67 or SEQ ID NO:220 in that the amino acid sequence of the variant comprises 1 to 6 individual amino acid substitutions,
with the proviso that the residue at position 35 of said SEQ ID NO:8, SEQ ID NO:9, SEQ ID NO:34, SEQ ID NO:37, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 or variant thereof is a Tryptophan (W).

In another embodiment of the present disclosure, the polypeptide comprises a repeat region 2, comprising an amino acid sequence SEQ ID NO: 10, SEQ ID NO: 11, SEQ ID NO: 35, SEQ ID NO: 38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO: 68 or SEQ ID NO:221, or a functional variant thereof wherein the amino acid sequence of said variant differs from SEQ ID NO: 10, SEQ ID NO:11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO: 68 or SEQ ID NO:221 in that the amino acid sequence of the variant comprises 1 to 6 individual amino acid substitutions,
with the proviso that the residue at position 32 of said variant of SEQ ID NO: 10, SEQ ID NO: 11, SEQ ID NO:35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO:68 or SEQ ID NO:221 is a Tryptophan (W).

In another embodiment of the present disclosure, the polypeptide comprises a repeat region 3, comprising an amino acid sequence SEQ ID NO: 12, SEQ ID NO: 13, SEQ ID NO: 36, SEQ ID NO: 39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO:66 or SEQ ID NO:69, SEQ ID NO:222, or a functional variant thereof wherein the amino acid sequence of said variant differs from SEQ ID NO: 12, SEQ ID NO: 13, SEQ ID NO: 36, SEQ ID NO: 39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO:66 or SEQ ID NO:69, SEQ ID NO:222 in that the amino acid sequence of the variant comprises 1 to 6 individual amino acid substitutions,
with the proviso that the residue at position 36 of said variant of SEQ ID NO:12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66 or SEQ ID NO:69, SEQ ID NO:222 is a Tryptophan (W).

In one embodiment of the present disclosure, each repeat region of the polypeptide comprises, in addition to a Tryptophan (W) residue at position 35 of R1, position 32 of R2 or position 36 of R3, also a Glutamic Acid at position 36 of R1, position 33 of R2 or position 37 of R3, and/or a Phenylalanine at position 38 of R1, position 35 of R2, or position 39 of R3, preferably both a Glutamic Acid at position 36 of R1, position 33 of R2 or position 37 of R3 and a Phenylalanine at position 38 of R1, position 35 of R2, or position 39 of R3, based on the amino acid numbering provided herein. Said Glutamic Acid and Phenylalanine residues are conserved and play an important role in the binding between the repeat regions that comprises them and a polysaccharide or disaccharide. This can also be seen in FIG. 3B. The Tryptophan, Glutamic Acid and/or Phenylalanine could be present at the designated positions as described above at one, two or all three repeat regions.

Thus, in a further embodiment of the present disclosure, the polypeptide comprises a repeat region 1, comprising an amino acid sequence SEQ ID NO:8, SEQ ID NO:9, SEQ ID NO:34, SEQ ID NO:37, SEQ ID NO: 40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220, or a functional variant thereof wherein the amino acid sequence of said variant differs from SEQ ID NO:8, SEQ ID NO:9, SEQ ID NO: 34, SEQ ID NO:37, SEQ ID NO:40, SEQ ID NO: 43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 in that the amino acid sequence of the variant comprises 1 to 6 individual amino acid substitutions,
with the proviso that
the residue at position 35 of said variant of SEQ ID SEQ ID NO:8, SEQ ID NO:9, SEQ ID NO: 34, SEQ ID NO:37, SEQ ID NO:40, SEQ ID NO:43, SEQ ID NO: 46, SEQ ID NO: 49, SEQ ID NO: 52, SEQ ID NO: 55, SEQ ID NO:58, SEQ ID NO:61, SEQ ID NO: 64, SEQ ID NO:67 or SEQ ID NO:220 is a Tryptophan (W), and
the residue at position 36 of said variant of SEQ ID NO:8, SEQ ID NO:9, SEQ ID NO: 34, SEQ ID NO:37, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 is a Glutamic acid (E), and/or
the residue at position 38 of said variant of SEQ ID NO:8, SEQ ID NO:9, SEQ ID NO: 34, SEQ ID NO: 37, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 is a Phenylalanine (F).

In another embodiment of the present disclosure, the polypeptide comprises a repeat region 2, comprising an amino acid sequence SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO: 68 or SEQ ID NO:221, or a functional variant thereof wherein the amino acid sequence of said variant differs from SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO:68 or SEQ ID NO:221 in that the amino acid sequence of the variant comprises 1 to 6 individual amino acid substitutions,
with the proviso that
the residue at position 32 of said variant of SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO:68 or SEQ ID NO:221 or is a Tryptophan (W),
the residue at position 33 of said variant of SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO:68 or SEQ ID NO:221 is a Glutamic acid (E), and/or
the residue at position 35 of said variant of SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO:68 or SEQ ID NO:221 a Phenylalanine (F).

In another embodiment of the present disclosure, the polypeptide comprises a repeat region 3, comprising an amino acid sequence SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO: 69 or SEQ ID NO:222, or a functional variant thereof wherein the amino acid sequence of said variant differs from SEQ ID NO: 12, SEQ ID NO:13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO: 69 or SEQ ID NO:222 in that the amino acid sequence of the variant comprises 1 to 6 individual amino acid substitutions,
with the proviso that
the residue at position 36 of said variant of SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69 or SEQ ID NO:222 is a Tryptophan (W),
the residue at position 37 of said variant of SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69 or SEQ ID NO:222 is a Glutamic acid (E), and/or
the residue at position 39 of said variant of SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69 or SEQ ID NO:222 is a Phenylalanine (F).

In one embodiment of the present disclosure, each repeat region of the polypeptide comprises a Tyrosine at position 12 of R1 or at position 11 of R2 and R3 and/or a Valine at position 13 of R1 or at position 12 of R2 and R3 based on the amino acid numbering provided herein. These Tyrosine and/or Valine residues may contribute to binding of a repeat region to a disaccharide or polysaccharide, as they are located in proximity of the binding site in the 3D-structure of the polypeptide, when it binds to a disaccharide or polysaccharide.

In a further embodiment of the present disclosure, the polypeptide comprises a repeat region 1, comprising an amino acid sequence SEQ ID NO:8, SEQ ID NO:9, SEQ ID NO: 34, SEQ ID NO:37, SEQ ID NO:40, SEQ ID NO:43, SEQ ID NO:46, SEQ ID NO: 49, SEQ ID NO: 52, SEQ ID NO: 55, SEQ ID NO:58, SEQ ID NO:61, SEQ ID NO: 64, SEQ ID NO:67 or SEQ ID NO:220, or a functional variant thereof wherein the amino acid sequence of said variant differs from SEQ ID NO:8, SEQ ID NO:9, SEQ ID NO: 34, SEQ ID NO:37, SEQ ID NO: 40, SEQ ID NO: 43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 in that the amino acid sequence of the variant comprises 1 to 6 individual amino acid substitutions,
with the proviso that
the residue at position 12 of said variant of SEQ ID NO:8, SEQ ID NO:9, SEQ ID NO: 34, SEQ ID NO: 37, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 is a Tyrosine (Y), and/or the residue at position 13 of said variant of SEQ ID NO:8, SEQ ID NO:9, SEQ ID NO: 34, SEQ ID NO: 37, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 is a Valine (V).

In another embodiment of the present disclosure, the polypeptide comprises a repeat region 2, comprising an amino acid sequence SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO: 53, SEQ ID NO: 56, SEQ ID NO:59, SEQ ID NO:62, SEQ ID NO: 65, SEQ ID NO:68 or SEQ ID NO:221, or a functional variant thereof wherein the amino acid sequence of said variant differs from SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO: 68 or SEQ ID NO:221 in that the amino acid sequence of the variant comprises 1 to 6 individual amino acid substitutions,
with the proviso that
the residue at position 11 of said variant of SEQ ID NO: 10, SEQ ID NO: 11, SEQ ID NO: 35, SEQ ID NO: 38, SEQ ID NO:41, SEQ ID NO:44, SEQ ID NO:47, SEQ ID NO: 50, SEQ ID NO: 53, SEQ ID NO: 56, SEQ ID NO:59, SEQ ID NO:62, SEQ ID NO: 65, SEQ ID NO:68 or SEQ ID NO:221 is a Tyrosine (Y), and/or
the residue at position 12 of said variant of SEQ ID NO: 10, SEQ ID NO: 11, SEQ ID NO: 35, SEQ ID NO: 38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO:68 or SEQ ID NO:221 is a Valine (V).

In another embodiment of the present disclosure, the polypeptide comprises a repeat region 2, comprising an amino acid sequence SEQ ID NO: 12, SEQ ID NO: 13, SEQ ID NO: 36, SEQ ID NO: 39, SEQ ID NO: 42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO: 69, SEQ ID NO:222, or a functional variant thereof wherein the amino acid sequence of said variant differs from SEQ ID NO: 12, SEQ ID NO:13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69, SEQ ID NO:222 in that the amino acid sequence of the variant comprises 1 to 6 individual amino acid substitutions,
with the proviso that
the residue at position 11 of said variant of SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69, SEQ ID NO:222 is a Tyrosine (Y), and/or
the residue at position 12 of said variant of SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69, SEQ ID NO:222 is a Valine (V).

In one embodiment of the present disclosure, the repeat region 1 of the polypeptide disclosed herein comprises or consists of an amino acid sequence SEQ ID NO:8, SEQ ID NO: 9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67, SEQ ID NO:220, or a functional variant thereof wherein the amino acid sequence of said variant differs from SEQ ID NO:8, SEQ ID NO: 9, SEQ ID NO:34, SEQ ID NO:37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO: 64, SEQ ID NO:67, SEQ ID NO:220, in that the amino acid sequence of the variant comprises 1 to 6 individual amino acid substitutions.

In one embodiment of the present disclosure, the repeat region 2 comprises or consists of an amino acid sequence SEQ ID NO: 10, SEQ ID NO: 11, SEQ ID NO:35, SEQ ID NO: 38, SEQ ID NO: 41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65, SEQ ID NO: 68, SEQ ID NO:221, or a functional variant thereof wherein the amino acid sequence of said variant differs from SEQ ID NO:10, SEQ ID NO:11, SEQ ID NO:35, SEQ ID NO:38, SEQ ID NO:41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65, SEQ ID NO: 68, SEQ ID NO:221 in that the amino acid sequence of the variant comprises 1 to 6 individual amino acid substitutions.

In one embodiment of the present disclosure, the repeat region 3 comprises or consists of an amino acid sequence SEQ ID NO: 12, SEQ ID NO: 13, SEQ ID NO:36, SEQ ID NO: 39, SEQ ID NO: 42, SEQ ID NO: 45, SEQ ID NO: 48, SEQ ID NO:51, SEQ ID NO: 54, SEQ ID NO:57, SEQ ID NO:60, SEQ ID NO:63, SEQ ID NO:66, SEQ ID NO: 69, SEQ ID NO:222 or a functional variant thereof wherein the amino acid sequence of said variant differs from SEQ ID NO:12, SEQ ID NO:13, SEQ ID NO:36, SEQ ID NO:39, SEQ ID NO:42, SEQ ID NO:45, SEQ ID NO:48, SEQ ID NO:51, SEQ ID NO: 54, SEQ ID NO:57, SEQ ID NO:60, SEQ ID NO:63, SEQ ID NO:66, SEQ ID NO: 69, SEQ ID NO:222, in that the amino acid sequence of the variant comprises 1 to 6 individual amino acid substitutions.

In one embodiment of the present disclosure, each of repeat region 1, repeat region 2 and repeat region 3 is flanked by at least three amino acid residues at its N-terminus and at least three amino acid residues at its C-terminus.

For example, in the polypeptide of the present disclosure each of repeat region 1, repeat region 2 and repeat region 3 is flanked by at least three amino acid residues at its N-terminus and at least three amino acid residues at its C-terminus so that:

Y_{N}—R1-Y_{C}Z_{N}—R2-Z_{C}J_{N}-R3-J_{C},
wherein each of
Y_{N}, Y_{C}, Z_{N}, Z_{C}, J_{N}, and J_{C} can individually be an amino acid sequence comprising at least three amino acids.

This structure is only one example and each of Y_{N}, Y_{C}, Z_{N}, Z_{C}, J_{N}, and J_{C} can individually be an amino acid sequence comprising more than 3 amino acids, such as more than, 5 amino acids, such as more than 10 amino acids, such as more than 15 amino acids, such as more than 20 amino acids. Furthermore, the number of amino acids in each of Y_{N}, Y_{C}, Z_{N}, Z_{C}, J_{N}, and J_{C} may be the same or different.

In one embodiment of the present disclosure, the functional variant SEQ ID NO:8, SEQ ID NO: 9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67 or SEQ ID NO:220 differs from SEQ ID NO:8, SEQ ID NO:9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO: 64, SEQ ID NO:67 or SEQ ID NO:220, in that the amino acid sequence of the variant comprises 5 individual amino acid substitutions, such as 4 individual amino acid substitutions, such as 3 individual amino acid substitutions, such as 2 individual amino acid substitutions, such as 1 individual amino acid substitution.

In one embodiment of the present disclosure, the functional variant of SEQ ID NO:8, SEQ ID NO:9, SEQ ID NO:34, SEQ ID NO:37, SEQ ID NO:40, SEQ ID NO:43, SEQ ID NO: 46, SEQ ID NO: 49, SEQ ID NO:52, SEQ ID NO:55, SEQ ID NO:58, SEQ ID NO: 61, SEQ ID NO:64, SEQ ID NO:67 or SEQ ID NO:220 differs from SEQ ID NO:8, SEQ ID NO:9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67 or SEQ ID NO:220 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 34 or 36 to 38 of SEQ ID NO:8, SEQ ID NO:9, SEQ ID NO:34, SEQ ID NO:37, SEQ ID NO:40, SEQ ID NO: 43, SEQ ID NO:46, SEQ ID NO:49, SEQ ID NO:52, SEQ ID NO:55, SEQ ID NO: 58, SEQ ID NO:61, SEQ ID NO:64, SEQ ID NO:67 or SEQ ID NO:220.

In one embodiment of the present disclosure, the functional variant of SEQ ID NO:8, SEQ ID NO:9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67 or SEQ ID NO:220 differs from SEQ ID NO:8, SEQ ID NO:9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO: 64, SEQ ID NO:67 or SEQ ID NO:220 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 34 or 37 of SEQ ID NO:8, SEQ ID NO:9, SEQ ID NO:34, SEQ ID NO:37, SEQ ID NO:40, SEQ ID NO: 43, SEQ ID NO: 46, SEQ ID NO:49, SEQ ID NO:52, SEQ ID NO:55, SEQ ID NO: 58, SEQ ID NO:61, SEQ ID NO:64, SEQ ID NO:67 or SEQ ID NO:220.

In one embodiment of the present disclosure, the functional variant of SEQ ID NO: 10, SEQ ID NO: 11, SEQ ID NO: 35, SEQ ID NO: 38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO:68 or SEQ ID NO:221, differs from SEQ ID NO: 10, SEQ ID NO: 11, SEQ ID NO:35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO: 68 or SEQ ID NO:221 in that the amino acid sequence of the variant comprises 5 individual amino acid substitutions, such as 4 individual amino acid substitutions, such as 3 individual amino acid substitutions, such as 2 individual amino acid substitutions, such as 1 individual amino acid substitution.

In one embodiment of the present disclosure, the functional variant of SEQ ID NO:10, SEQ ID NO: 11, SEQ ID NO:35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO:68 or SEQ ID NO:221 differs from SEQ ID NO: 10, SEQ ID NO: 11, SEQ ID NO:35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO:68 or SEQ ID NO:221 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 31 or 33 to 35 of SEQ ID NO: 10, SEQ ID NO: 11, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO: 59, SEQ ID NO:62, SEQ ID NO:65, SEQ ID NO:68 or SEQ ID NO:221.

In one embodiment of the present disclosure, the functional variant of SEQ ID NO:10, SEQ ID NO: 11, SEQ ID NO:35, SEQ ID NO: 38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO: 65, SEQ ID NO:68 or SEQ ID NO:221 differs from SEQ ID NO: 10, SEQ ID NO: 11, SEQ ID NO:35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO: 65, SEQ ID NO:68 or SEQ ID NO:221 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 31 or 34 of SEQ ID NO: 10, SEQ ID NO: 11, SEQ ID NO:35, SEQ ID NO:38, SEQ ID NO:41, SEQ ID NO: 44, SEQ ID NO:47, SEQ ID NO:50, SEQ ID NO:53, SEQ ID NO:56, SEQ ID NO: 59, SEQ ID NO:62, SEQ ID NO:65, SEQ ID NO:68 or SEQ ID NO:221.

In one embodiment of the present disclosure, the functional variant of SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO:69 or SEQ ID NO:222 differs from SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO: 69 or SEQ ID NO:222 in that the amino acid sequence of the variant comprises 5 individual amino acid substitutions, such as 4 individual amino acid substitutions, such as 3 individual amino acid substitutions, such as 2 individual amino acid substitutions, such as 1 individual amino acid substitution.

In one embodiment of the present disclosure, the functional variant of SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO:69 or SEQ ID NO:222 differs from SEQ ID NO: 12, SEQ ID NO: 13, SEQ ID NO: 36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO: 66, SEQ ID NO: 69 or SEQ ID NO:222 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 35 or 37 to 39 of SEQ ID NO: 12, SEQ ID NO: 13, SEQ ID NO:36, SEQ ID NO:39, SEQ ID NO:42, SEQ ID NO:45, SEQ ID NO:48, SEQ ID NO. 51, SEQ ID NO. 54, SEQ ID NO. 57, SEQ ID NO: 60, SEQ ID NO:63, SEQ ID NO:66, SEQ ID NO:69 or SEQ ID NO:222.

In one embodiment of the present disclosure, the functional variant of SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO: 66, SEQ ID NO:69 or SEQ ID NO:222 differs from SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO: 66, SEQ ID NO: 69 or SEQ ID NO:222 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 35 or 38 of SEQ ID NO: 12, SEQ ID NO: 13, SEQ ID NO:36, SEQ ID NO:39, SEQ ID NO:42, SEQ ID NO: 45, SEQ ID NO:48, SEQ ID NO. 51, SEQ ID NO. 54, SEQ ID NO. 57, SEQ ID NO: 60, SEQ ID NO:63, SEQ ID NO:66, SEQ ID NO:69 or SEQ ID NO:222.

In one embodiment of the present disclosure, the individual amino acid substitutions are conservative amino acid substitutions.

In one embodiment of the present disclosure, the repeat region 1 of the polypeptide described herein comprises or consists of an amino acid sequence of SEQ ID NO:8, SEQ ID NO:9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67.

In one embodiment of the present disclosure, the repeat region 2 of the polypeptide described herein comprises or consists of an amino acid sequence of SEQ ID NO:10, SEQ ID NO: 11, SEQ ID NO:35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO:68.

In one embodiment of the present disclosure, the repeat region 3 of the polypeptide described herein comprises or consists of an amino acid sequence of SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO:69.

In one embodiment of the present disclosure, the polypeptide comprises repeat region 1 and repeat region 2, or functional variants thereof; repeat region 1 and repeat region 3, or functional variants thereof; repeat region 2 and repeat region 3, or functional variants thereof; repeat region 1, repeat region 2 and repeat region 3, or functional variants thereof.

In a further aspect the present disclosure relates to a polypeptide comprising an amino acid sequence having at least 75% sequence identity to SEQ ID NO:3, SEQ ID NO:4, SEQ ID NO:20 to SEQ ID NO:33, or SEQ ID NO:71 to 219, and wherein said polypeptide is capable of binding one or more saccharide units.

In one embodiment of the present disclosure relates to a polypeptide comprising an amino acid sequence having at least 75% sequence identity to SEQ ID NO:3, SEQ ID NO: 4, SEQ ID NO:20 to SEQ ID NO:33, and wherein said polypeptide is capable of binding one or more saccharide units.

In a further embodiment of the present disclosure, the polypeptide comprises an amino acid sequence having at least 80% sequence identity to SEQ ID NO:3, SEQ ID NO:4 or SEQ ID NO:20 to SEQ ID NO:33, such as at least 85% sequence identity to SEQ ID NO: 3, SEQ ID NO:4 or SEQ ID NO:20 to SEQ ID NO:33, such as at least 90% sequence identity to SEQ ID NO:3, SEQ ID NO:4 or SEQ ID NO:20 to SEQ ID NO:33, such as at least 95% sequence identity to SEQ ID NO:3, SEQ ID NO:4 or SEQ ID NO: 20 to SEQ ID NO:33, such as at least 96% sequence identity to SEQ ID NO:3, SEQ ID NO:4 or SEQ ID NO:20 to SEQ ID NO:33, such as at least 97% sequence identity to SEQ ID NO:3, SEQ ID NO:4 or SEQ ID NO:20 to SEQ ID NO:33, such as at least 98% sequence identity to SEQ ID NO:3, SEQ ID NO:4 or SEQ ID NO:20 to SEQ ID NO: 33, such as at least 99% sequence identity to SEQ ID NO:3, SEQ ID NO:4 or SEQ ID NO:20 to SEQ ID NO:33.

In another embodiment of the present disclosure, the polypeptide comprises or consists of amino acid sequence SEQ ID NO:3, SEQ ID NO:4 or SEQ ID NO:20 to SEQ ID NO:33.

In one embodiment of the present disclosure, the polypeptide comprises at least two repeat regions selected from the group consisting of:
a. a repeat region 1, comprising or consisting of an amino acid sequence of SEQ ID NO:8, SEQ ID NO:9, SEQ ID NO:34, SEQ ID NO:37, SEQ ID NO: 40, SEQ ID NO:43, SEQ ID NO:46, SEQ ID NO:49, SEQ ID NO:52, SEQ ID NO:55, SEQ ID NO:58, SEQ ID NO:61, SEQ ID NO:64 or SEQ ID NO: 67 or a functional variant thereof wherein the amino acid sequence of said variant differs from SEQ ID NO:8, SEQ ID NO:9, SEQ ID NO:34, SEQ ID NO: 37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO: 64 or SEQ ID NO:67 in that the amino acid sequence of the variant comprises 1 to 6 individual amino acid substitutions;
b. a repeat region 2, comprising or consisting of an amino acid sequence of SEQ ID NO:10, SEQ ID NO: 11, SEQ ID NO:35, SEQ ID NO:38, SEQ ID NO: 41, SEQ ID NO: 44, SEQ ID NO: 47, SEQ ID NO:50, SEQ ID NO: 53, SEQ ID NO:56, SEQ ID NO:59, SEQ ID NO:62, SEQ ID NO:65 or SEQ ID NO: 68 or a functional variant thereof wherein the amino acid sequence of said variant differs from SEQ ID NO:10, SEQ ID NO:11, SEQ ID NO:35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO:65 or SEQ ID NO:68 in that the amino acid sequence of the variant comprises 1 to 6 individual amino acid substitutions; and
c. a repeat region 3, comprising or consisting of an amino acid sequence of SEQ ID NO: 12, SEQ ID NO: 13, SEQ ID NO:36, SEQ ID NO:39, SEQ ID NO: 42, SEQ ID NO:45, SEQ ID NO:48, SEQ ID NO:51, SEQ ID NO:54, SEQ ID NO:57, SEQ ID NO:60, SEQ ID NO:63, SEQ ID NO:66 or SEQ ID NO: 69, or a functional variant thereof wherein the amino acid sequence of said variant differs from SEQ ID NO: 12, SEQ ID NO: 13, SEQ ID NO:36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO:66, SEQ ID NO:69 in that the amino acid sequence of the variant comprises 1 to 6 individual amino acid substitutions.

In one embodiment of the present disclosure, the polypeptide comprises three repeat regions each selected from the group consisting of:
a. a repeat region 1, comprising or consisting of an amino acid sequence of SEQ ID NO:8, SEQ ID NO:9, SEQ ID NO:34, SEQ ID NO:37, SEQ ID NO: 40, SEQ ID NO:43, SEQ ID NO:46, SEQ ID NO:49, SEQ ID NO:52, SEQ ID NO:55, SEQ ID NO:58, SEQ ID NO:61, SEQ ID NO:64 or SEQ ID NO: 67 or a functional variant thereof wherein the amino acid sequence of said variant differs from SEQ ID NO:8, SEQ ID NO:9, SEQ ID NO: 34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO:61, SEQ ID NO: 64 or SEQ ID NO:67 in that the amino acid sequence of the variant comprises 1 to 6 individual amino acid substitutions;
b. a repeat region 2, comprising or consisting of an amino acid sequence of SEQ ID NO: 10, SEQ ID NO: 11, SEQ ID NO:35, SEQ ID NO:38, SEQ ID NO: 41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65 or SEQ ID NO:68 or a functional variant thereof wherein the amino acid sequence of said variant differs from SEQ ID NO:10, SEQ ID NO:11, SEQ ID NO:35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO:62, SEQ ID NO:65 or SEQ ID NO:68 in that the amino acid sequence of the variant comprises 1 to 6 individual amino acid substitutions; and
c. a repeat region 3, comprising or consisting of an amino acid sequence of SEQ ID NO: 12, SEQ ID NO: 13, SEQ ID NO:36, SEQ ID NO:39, SEQ ID NO: 42, SEQ ID NO:45, SEQ ID NO:48, SEQ ID NO:51, SEQ ID NO: 54, SEQ ID NO:57, SEQ ID NO:60, SEQ ID NO:63, SEQ ID NO:66 or SEQ ID NO:69, or a functional variant thereof wherein the amino acid sequence of said variant differs from SEQ ID NO:12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO:63, SEQ ID NO:66, SEQ ID NO:69 in that the amino acid sequence of the variant comprises 1 to 6 individual amino acid substitutions.

In an embodiment of the present disclosure, the polypeptide has a beta trefoil fold structure. Preferably, the structure consists of six β hairpins, each formed by two β strands, showing approximate three-fold symmetry.

A function of the polypeptide of the present disclosure is that it can bind certain disaccharides and polysaccharides and it can even cross-link certain polysaccharides. This function is connected at least partially to the presence in the polypeptides of the present disclosure of two or preferably three repeat regions, such as repeat region 1, repeat region 2 and/or repeat region 3, which are regions capable of recognizing and binding disaccharides and polysaccharides and are defined herein.

Thus, in one embodiment of the present disclosure, each of repeat region 1, repeat region 2 and repeat region 3 is capable of binding at least one disaccharide.

In one embodiment of the present disclosure, each of repeat region 1, repeat region 2 and repeat region 3 is capable of binding at least one polysaccharide.

Despite the fact that they may have different amino acid sequences, the repeat regions of the polypeptides of the present disclosure are capable of binding disaccharides and polysaccharides characterized by comprising the same type of linkage between monosaccharide units.

The polypeptides of the present disclosure can bind any disaccharide or longer oligo- or polysaccharide as long as it comprises the specific Glc-β-1,6-linkage. A number of polysaccharides described herein comprise this linkage.

The polypeptides of the present disclosure also show binding to extremely rare Glc-β-1,2-Glc structures, which are found in unusual bacterial secretions.

In one embodiment of the present disclosure, each of repeat region 1, repeat region 2 and/or repeat region 3 is capable of binding a disaccharide or polysaccharide comprising at least one Glc-β-1,6-Glc unit.

In another embodiment of the present disclosure, each of repeat region 1, repeat region 2 and/or repeat region 3 is capable of binding a disaccharide or polysaccharide comprising at least one Glc-β-1,2-Glc unit.

In one embodiment of the present disclosure, each of repeat region 1, repeat region 2 and/or repeat region 3 is capable of binding a disaccharide or polysaccharide comprising a glycan comprising at least one Glc-β-1,6-Glc unit.

In one embodiment of the present disclosure, each of repeat region 1, repeat region 2 and/or repeat region 3 is capable of binding a disaccharide or polysaccharide having a β-1,6-glucan backbone.

In one embodiment of the present disclosure, each of repeat region 1, repeat region 2 and/or repeat region 3 is capable of binding a disaccharide or polysaccharide having one or more Glc-β-1,6-Glc decoration.

In one embodiment of the present disclosure, each of repeat region 1, repeat region 2 and/or repeat region 3 is capable of binding a disaccharide or polysaccharide having one or more Glc-β-1,6-Glc decoration(s) on a β-1,3-glucan backbone.

In one embodiment of the present disclosure, each of repeat region 1, repeat region 2 and/or repeat region 3 is capable of binding the polysaccharide scleroglucan.

In one embodiment of the present disclosure, each of repeat region 1, repeat region 2 and/or repeat region 3 is capable of binding the polysaccharide pustulan.

In one embodiment of the present disclosure, each of repeat region 1, repeat region 2 and/or repeat region 3 is capable of binding the polysaccharide laminarin.

In one embodiment of the present disclosure, each of repeat region 1, repeat region 2 and/or repeat region 3 is capable of binding the polysaccharide gentiobiose.

In one embodiment of the present disclosure, the saccharide is at least a disaccharide.

In one embodiment of the present disclosure, the saccharide is an oligosaccharide or a polysaccharide.

### Polynucleotides Encoding Carbohydrate Binding Polypeptides

In one aspect the present disclosure relates to a polynucleotide encoding a polypeptide described herein.

In one embodiment of the present disclosure the polynucleotide encodes a polypeptide comprising at least two repeat regions selected from the group consisting of:
a. a repeat region 1, comprising or consisting of an amino acid sequence SEQ ID NO: 220:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈ X₂₉ X₃₀X₃₁X₃₂X₃₃X₃₄WEX₃₇F,
wherein
   X₁ is Isoleucine (I) or Valine (V);
   X₂ is any amino acid, such as Tryptophan (W), Serine (S), Threonine (T) or Alanine (A);
   X₃ is Leucine (L) or Isoleucine (I);
   X₄ is Lysine (K), or Arginine (R), or is omitted;
   X₅ is Glutamine (Q), Arginine (R), Alanine (A) or Lysine (K), or omitted;
   X₆ is Glycine (G), Serine (S), Tyrosine (Y), Cysteine (C), Leucine (L) or Threonine (T);
   X₇ is any amino acid, such as Phenylalanine (F) or Serine (S);
   X₈ is Glycine (G), or is omitted;
   X₉ is Asparagine (N), Histidine (H), Aspartic acid (D) or Threonine (T);
   X₁₀ is Asparagine (N), Serine (S), Threonine (T), Glutamine (Q), Lysine (K) or Glycine (G);
   X₁₁ is Lysine (K), Glutamine (Q), Asparagine (N) or Leucine (L);
   X₁₂ is Tyrosine (Y), Leucine (L) or Phenylalanine (F);
   X₁₃ is Valine (V), Leucine (L) or Alanine (A);
   X₁₄ is Serine (S), Threonine (T), Cysteine (C) or Asparagine (N);
   X₁₅ is Alanine (A), Serine (S) or Glycine (G);
   X₁₆ is Glutamic acid (E), Lysine (K), Arginine (R), Glutamine (Q) or Aspartic acid (D);
   X₁₇ is Asparagine (N), Glycine (G), Glutamine (Q) or Proline (P);
   X₁₈ is Glycine (G), Asparagine (N), Threonine (T) or Aspartic acid (D);
   X₁₉ is Leucine (L) or is omitted;
   X₂₀ is Glycine (G) or Alanine (A), or is omitted;
   X₂₁ is Alanine (A) or Asparagine (N), or is omitted;
   X₂₂ is any amino acid, such as Threonine (T), or is omitted;
   X₂₃ is any amino acid, such as Glycine (G);
   X₂₄ is Proline (P), Alanine (A), Glutamine (Q) or Arginine (R);
   X₂₅ is Leucine (L), Methionine (M), Alanine (A) or Isoleucine (I);
   X₂₆ is any amino acid, such as Threonine (T);
   X₂₇ is Alanine (A), Cysteine (C) or Tryptophan (W);
   X₂₈ is Asparagine (N), Aspartic acid (D), Glutamic acid (E) or Arginine (R);
   X₂₉ is Arginine (R), Alanine (A) or Serine (S);
   X₃₀ is Threonine (T), Aspartic acid (D), Proline (P), Isoleucine (I), Asparagine (N) or Alanine (A);
   X₃₁ is Threonine (T), Alanine (A), Valine (V), Glycine (G), Lysine (K) or Isoleucine (I);
   X₃₂ is Alanine (A), Valine (V), Proline (P) or Leucine (L);
   X₃₃ is Glycine (G), Glutamine (Q), Serine (S) or Aspartic acid (D);
   X₃₄ is any amino acid, such as Glycine (G);
   X₃₇ is any amino acid, such as Glutamine (Q) or Lysine (K); or a functional variant thereof wherein the amino acid sequence of said variant differs from SEQ ID NO: 220 in that the amino acid sequence of the variant comprises 1 to 6 individual amino acid substitutions,
b. a repeat region 2, comprising or consisting of an amino acid sequence SEQ ID NO: 221:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈ X₂₉ X₃₀X₃₁ WEX₃₄F,
wherein
   X₁ is Isoleucine (I) or Valine (V);
   X₂ is Alanine (A), Asparagine (N), Tyrosine (Y) or Threonine (T);
   X₃ is Leucine (L) or Isoleucine (I);
   X₄ is Glutamine (Q), Arginine (R), Leucine (L) or Lysine (K);
   X₅ is Glycine (G), Alanine (A), Asparagine (N) or Serine (S);
   X₆ is any amino acid, such as Alanine (A), or is omitted;
   X₇ is Glycine (G) or Serine (S), or is omitted;
   X₈ is Asparagine (N), Methionine (M), Glutamine (Q), Histidine (H) or Serine (S);
   X₉ is Glycine (G) or Alanine (A);
   X₁₀ is Lysine (K), Leucine (L), Methionine (M), Asparagine (N) or Serine (S);
   X₁₁ is Tyrosine (Y);
   X₁₂ is Valine (V) or Phenylalanine (F);
   X₁₃ is Serine (S), Cysteine (C), Lysine (K) or Glutamine (Q);
   X₁₄ is Alanine (A), Valine (V) or Serine (S);
   X₁₅ is Glutamic acid (E), Aspartic acid (D), Glutamine (Q) or Glycine (G);
   X₁₆ is Asparagine (N), Proline (P), Glycine (G), Threonine (T) or Aspartic acid (D)
   X₁₇ is Glycine (G), Aspartic Acid (D), Isoleucine (I) or Asparagine (N), or is omitted;
   X₁₈ is Valine (V) or Glycine (G), or is omitted;
   X₁₉ is Glycine (G), Threonine (T), Leucine (L), Alanine (A), or Glutamic acid (E) or is omitted;
   X₂₀ is any amino acid, such as Glutamine (Q), or omitted;
   X₂₁ is Proline (P), Alanine (A), Glutamine (Q) or Asparagine (N);
   X₂₂ is Leucine (L), Isoleucine (I), Methionine (M) or Valine (V);
   X₂₃ is Threonine (T), Asparagine (N), Phenylalanine (F) or Arginine (R);
   X₂₄ is Alanine (A), Cysteine (C) or Arginine (R);
   X₂₅ is Asparagine (N), Alanine (A), Threonine (T) or Glutamine (Q);
   X₂₆ is Alanine (A), Glycine (G) or Arginine (R);
   X₂₇ is Threonine (T), Alanine (A), Lysine (K), Proline (P) or Leucine (L);
   X₂₈ is any amino acid, such as Alanine (A);
   X₂₉ is Isoleucine (I), Valine (V), Proline (P), Alanine (A), Lysine (K), Tyrosine (Y) or Leucine (L);
   X₃₀ is Glycine (G), Alanine (A), Glutamic acid (E), Threonine (T), Serine (S), Glutamine (Q) or Aspartic acid (D);
   X₃₁ is Glycine (G), Alanine (A), Aspartic Acid (D), Serine (S), Proline (P) or Lysine (K);
   X₃₄ is any amino acid, such as Lysine (K);
or a functional variant thereof wherein the amino acid sequence of said variant differs from SEQ ID NO:221 in that the amino acid sequence of the variant comprises 1 to 6 individual amino acid substitutions,
c. a repeat region 3, comprising or consisting of an amino acid sequence SEQ ID NO: 222:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈ X₂₉ X₃₀X₃₁X₃₂X₃X₃₄X₃₅WEX₃₈F,
wherein
   X₁ is Valine (V), Isoleucine (I), Tyrosine (Y) or Phenylalanine (F);
   X₂ is Alanine (A), Serine (S), Cysteine (C), Threonine (T) or Glycine (G);
   X₃ is Leucine (L), Phenylalanine (F) or Isoleucine (I);
   X₄ is Arginine (R), Lysine (K) or Glutamine (Q),
   X₅ is Glycine (G), Alanine (A) or Serine (S);
   X₆ is Arginine (R) or Valine (V), or is omitted;
   X₇ is any amino acid, such as Asparagine (N);
   X₈ is Asparagine (N), Histidine (H), Alanine (A), Lysine (K), or omitted;
   X₉ is Glycine (G), Tryptophan (W) or Asparagine (N);
   X₁₀ is Lysine (K), Leucine (L), Methionine (M), Serine (S), Alanine (A) or Glutamine (Q);
   X₁₁ is Tyrosine (Y), Tryptophan (W) or Phenylalanine (F);
   X₁₂ is Valine (V), Leucine (L), Methionine (M) or Isoleucine (I);
   X₁₃ is Serine (S) or Glutamine (Q);
   X₁₄ is Alanine (A), Serine (S), Histidine (H) or Glycine (G);
   X₁₅ is Glutamic acid (E), Aspartic acid (D), Glutamine (Q), Serine (S) or Asparagine (N);
   X₁₆ is Asparagine (N), Leucine (L), Tryptophan (W), Methionine (M) or Aspartic acid (D);
   X₁₇ is Glycine (G), Serine (S) or Aspartic Acid (D);
   X₁₈ is any amino acid, such as Serine (S) or Threonine (T);
   X₁₉ is Asparagine (N), Valine (V) or Aspartic acid (D), or is omitted;
   X₂₀ is Alanine (A), or is omitted;
   X₂₁ is Serine (S), or is omitted;
   X₂₂ is any amino acid, such as Alanine (A), or is omitted;
   X₂₃ is Proline (P), Alanine (A), Arginine (R), Threonine (T) or Glycine (G);
   X₂₄ is Leucine (L), Methionine (M), Valine (V) or Isoleucine (I);
   X₂₅ is Threonine (T), Tyrosine (Y), Asparagine (N), Leucine (L) or Glutamine (Q);
   X₂₆ is Alanine (A), Cysteine (C), Phenylalanine (F) or Tryptophan (W);
   X₂₇ is Asparagine (N), Alanine (A), Threonine (T) or Serine (S);
   X₂₈ is Arginine (R) or Lysine (K);
   X₂₉ is Aspartic acid (D), or is omitted;
   X₃₀ is Threonine (T), Alanine (A), Proline (P), Serine (S) or Glycine (G);
   X₃₁ is any amino acid, such as Alanine (A);
   X₃₂ is Isoleucine (I), Glutamic acid (E), Proline (P), Alanine (A) or Valine (V);
   X₃₃ is Glycine (G), Lysine (K) or Aspartic acid (D), or is omitted;
   X₃₄ is Glycine (G), Serine (S), Lysine (K), Aspartic acid (D) or Glutamine (Q);
   X₃₅ is Glycine (G), Alanine (A), Aspartic acid (D) or Cysteine (C);
   X₃₈ is any amino acid, such as Lysine (K);
or a functional variant thereof wherein the amino acid sequence of said variant differs from SEQ ID NO:222 in that the amino acid sequence of the variant comprises 1 to 6 individual amino acid substitutions.

In one embodiment of the present disclosure the polynucleotide is codon-optimized for expression in a host cell.

In another aspect the present disclosure relates to a recombinant vector comprising a polynucleotide described herein, or a nucleic acid sequence encoding a carbohydrate-binding polypeptide according to any of the embodiments described herein. Examples of a recombinant vector include but not limited to plasmids, viral vectors, cosmids, and artificial chromosomes.

In one embodiment of the present disclosure the vector is a plasmid vector.

In a further aspect the present disclosure relates to a host cell (e.g. standard expression strains of E. coli) expressing the recombinant vector as described herein.

### Hydrogels

As provided herein, the polypeptides of the present disclosure are capable of binding polysaccharides. Each polypeptide of the present disclosure can bind up to three chains of polysaccharides. Moreover, if several polypeptides of the present disclosure are present together with the polysaccharides, the polypeptides of the present disclosure may be capable of not only binding said polysaccharides but also cross-linking them. The polypeptides of the present disclosure are thus capable of binding and cross-linking certain polysaccharides; the specific polysaccharides are described herein.

In one aspect the present disclosure relates to use of the polypeptide described in any of the embodiments herein for cross-linking two or more polysaccharides.

Another aspect of the present disclosure relates to a composition comprising at least one polypeptide according to any of the embodiments described herein and two or more polysaccharides, wherein the polysaccharides are cross-linked by the polypeptide, such as wherein the polypeptide is the cross-linker linking the polysaccharides together.

In another aspect the present disclosure relates to a hydrogel comprising at least one polypeptide according to any of the embodiments described herein and two or more polysaccharides, wherein said hydrogel has a cross-linked structure.

In another aspect the present disclosure relates to a hydrogel comprising at least two polypeptides according to any of the embodiments described herein and three or more polysaccharides, wherein said hydrogel has a cross-linked structure.

Preferably, the present disclosure also relates to a hydrogel comprising a plurality of polypeptides according to any of the embodiments described herein and a plurality of polysaccharides, wherein said hydrogel has a cross-linked structure.

In fact, the polypeptides according to any of the embodiments described herein function as cross-linkers in the hydrogel of the present disclosure.

In one embodiment of the present disclosure, the hydrogel is cross-linked with a bi- or multifunctional linker, for example a linker capable of binding to two or more polysaccharide chains, which is a polypeptide as described herein.

In one embodiment of the present disclosure, the hydrogel comprises one or more polysaccharides each having a size of 20.000 Da or more, such as of 50.000 Da or more, such as of 70.000 Da or more, such as of 100.000 Da or more, such as of 150.000 Da or more, such as of 200.000 Da or more, such as of 300.000 Da or more, such as of 500.000 Da or more, such as of 700.000 Da or more, such as of 800.000 Da or more, such as of 900.000 Da or more, such as of 1.000.000 Da or more, such as where the polysaccharide has a size of about 2.000.000 Da Preferably, the polysaccharide at least 100.000 Da, such as at least 500.000 Da, such as at least 1.000.000 Da, such as 2.000.000 Da.

In one embodiment of the present disclosure, the hydrogel comprises a polysaccharide that is water-dispersible. In a further embodiment, the polysaccharide has a moisture content of at least 70 μg water per mg of saccharide, preferably at least 74 μg water per mg of saccharide, such as 77 μg water per mg of saccharide.

In one embodiment of the present disclosure, the hydrogel comprises one or more polysaccharides, which are biopolymers, each comprising one or more β-1,3-glucan units with β-1,6-linked glucose decorations to the main chain, such as scleroglucan or schizophyllan.

In one embodiment of the present disclosure, the hydrogel comprises between 0.1 g/L and 10 g/L of a polypeptide of the present disclosure, and between 2 g/L and 10 g/L of a polysaccharide defined herein. Preferably, the hydrogel comprises between 0.15 g/L and 5 g/L of a polypeptide of the present disclosure, and between 3 g/L and 5 g/L of a polysaccharide defined herein.

In one embodiment of the present disclosure, the hydrogel comprises at least 0.1 g/L of the polypeptide of the present disclosure, such as at least 0.2 g/L polypeptide, such as at least 0.3 g/L polypeptide, such as at least 0.5 g/L polypeptide, such as at least 0.8 g/L polypeptide, such as at least 1 g/L polypeptide, such as at least 1.3 g/L polypeptide, such as at least 1.7 g/L polypeptide, such as at least 2 g/L polypeptide, such as at least 2.5 g/L polypeptide, such as at least 3 g/L polypeptide, such as at least 3.5 g/L polypeptide, such as at least 4 g/L polypeptide, such as at least 4.5 g/L polypeptide, such as at least 5 g/L polypeptide, such as at least 5.5 g/L polypeptide, such as at least 6 g/L polypeptide, such as at least 6.5 g/L polypeptide, such as at least 7 g/L polypeptide, such as at least 7.5 g/L polypeptide, such as at least 8 g/L polypeptide, such as at 8.5 g/L polypeptide, such as at least 9 g/L polypeptide, such as at least 9.5 g/L polypeptide, such as at least 10 g/L of a polypeptide of the present disclosure.

In one embodiment of the present disclosure, the hydrogel comprises at the most 10 g/L of a polypeptide of the present disclosure.

In one embodiment of the present disclosure, the hydrogel comprises at least 2 g/L polysaccharide, such as at least 3 g/L polysaccharide, and at the most 5 g/L polysaccharide, such as at the most 6 g/L polysaccharide, such as at the most 7 g/L polysaccharide, such as at the most 8 g/L polysaccharide, such as at the most 9 g/L polysaccharide, 10 g/L polysaccharide. Preferably, in an embodiment of the present disclosure, the hydrogel comprises at least 3 g/L polysaccharide, and at the most 5 g/L polysaccharide, wherein the polysaccharide comprises at least one Glc-β-1,6-Glc unit.

In one embodiment of the present disclosure, the hydrogel comprises at least 2.5 g/L polysaccharide, such as at least 3 g/L polysaccharide, such as at least 3.5 g/L polysaccharide, such as at least 4 g/L polysaccharide, such as at least 4.5 g/L polysaccharide, such as at least 5 g/L polysaccharide, such as at least 5.5 g/L polysaccharide, such as at least 6 g/L polysaccharide, such as at least 6.5 g/L polysaccharide, such as at least 7 g/L polysaccharide, such as at least 7.5 g/L polysaccharide, such as at least 8 g/L polysaccharide, such as at 8.5 g/L polysaccharide, such as at least 9 g/L polysaccharide, such as at least 9.5 g/L polysaccharide, such as at the most 10 g/L of a polysaccharide, wherein the polysaccharide comprises at least one Glc-β-1,6-Glc unit.

In one embodiment of the present disclosure, the hydrogel comprises at least 2 g/L Scleroglucan, such as at least 3 g/L Scleroglucan, and at the most 10 g/L

Scleroglucan, such as at the most 6 g/L Scleroglucan, such as at the most 7 g/L Scleroglucan, such as at the most 8 g/L Scleroglucan, such as at the most 9 g/L Scleroglucan. Preferably, in an embodiment of the present disclosure, the hydrogel comprises at least 3 g/L Scleroglucan, and at the most 5 g/L Scleroglucan.

In one embodiment of the present disclosure, the hydrogel comprises at least 2.5 g/L Scleroglucan, such as at least 3 g/L Scleroglucan, such as at least 3.5 g/L Scleroglucan, such as at least 4 g/L Scleroglucan, such as at least 4.5 g/L Scleroglucan, such as at least 5 g/L Scleroglucan, such as at least 5.5 g/L Scleroglucan, such as at least 6 g/L Scleroglucan, such as at least 6.5 g/L Scleroglucan, such as at least 7 g/L Scleroglucan, such as at least 7.5 g/L Scleroglucan, such as at least 8 g/L Scleroglucan, such as at 8.5 g/L Scleroglucan, such as at least 9 g/L polysaccharide, such as at least 9.5 g/L polysaccharide, such as at the most 10 g/L of a polysaccharide, wherein the polysaccharide comprises at least one Glc-β-1,6-Glc unit.

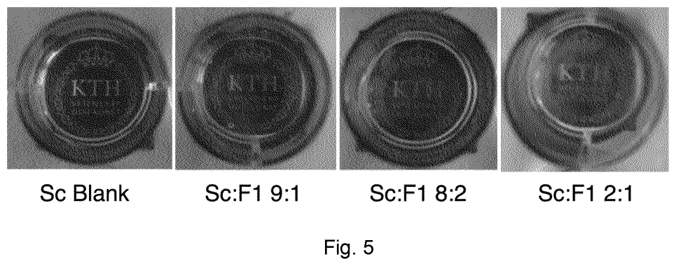

For example, as illustrated in FIG. 5 and example 5 of the present disclosure, a hydrogel can comprise 3 to 5 g/L of the polysaccharide Scleroglucan, such as about 4 g/L of the polysaccharide Scleroglucan.

In one embodiment of the present disclosure, the hydrogel comprises at least 90 wt. % water, such as at least 91 wt. % water, such as at least 92 wt. % water, such as at least 93 wt. % water, such as at least 94 wt. % water, such as at least 95 wt. % water, such as at least 96 wt. % water, such as at least 97 wt. % water, such as at least 98 wt. % water, such as at least 99 wt. % water, such as at least 99.5 wt. % water.

The hydrogel according to one embodiment of the present disclosure is able to dry out in air at room temperature and subsequently of being rehydrated, if put in contact with water. In one embodiment, the rehydrated gel has a lower water content that the original hydrogel, for example, the rehydrated gel may comprise 70 wt. % to 90 wt. % water. For example, the rehydrated hydrogel may be in the form of a film.

In one embodiment, the rehydrated gel has a water content that resembles that of the original hydrogel, for example, the rehydrated gel may comprise at least 70 wt. % water, such as at least 80 wt. % water, such as at least 90 wt. % water.

In an embodiment of the present disclosure the hydrogel can be dehydrated, retaining cross-linked structure, and re-hydrated or re-swelled again. By the processes of dehydration and rehydration is meant the process of alteration of the physical form of the hydrogel due to loss of water and restoration of water content, such as re-swelling, respectively.

Dehydration and rehydration of the hydrogel of the present disclosure may be conducted by any of the methods known in the art.

In an embodiment of the present disclosure, the hydrogel is a re-hydrated hydrogel and the re-hydrated hydrogel comprises at least 70 wt. % water, such as at least 73 wt. % water, such as at least 75 wt. % water, such as at least 78 wt. % water, such as at least 80 wt. % water, such as at least 83 wt. % water, such as at least 85 wt. % water, such as at least 88 wt. % water, such as at least 90 wt. % water.

In an embodiment of the present disclosure, the hydrogel comprises the polypeptide of the present disclosure and a polysaccharide at a ratio of polypeptide to polysaccharide between 1:20 to 1:2, such as at a ratio between 1:20 and 1:3, such as at a ratio between 1:20 and 1:5, such as at a ratio between 1:20 and 1:7, such as at a ratio between 1:20 and 1:10, such as at a ratio between 1:20 and 1:12, such as at a ratio between 1:20 and 1:15, such as at a ratio between 1:20 and 1:18, such as at a ratio between 1:18 and 1:2, such as at a ratio between 1:15 and 1:2, such as at a ratio between 1:13 and 1:2, such as at a ratio between 1:10 and 1:2, such as at a ratio between 1:8 and 1:2, such as at a ratio between 1:6 and 1:2, such as at a ratio between 1:4 and 1:2. For example, the polysaccharide may be scleroglucan. These ratios are volume:volume ratios based on a stock solution of polysaccharide of 5 g/L in water at room temperature, and a stock solution of a polypeptide of the present disclosure of 4 g/l in water at room temperature.

In an embodiment of the present disclosure, the hydrogel comprises:
a polypeptide comprising at least two repeat regions selected from the group consisting of:
   a. a repeat region 1 (R1), comprising or consisting of an amino acid sequence SEQ ID NO:220:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁X₃₂X₃₃X₃₄WEX₃₇F,
   wherein
   X₁ is Isoleucine (I) or Valine (V);
   X₂ is any amino acid, such as Tryptophan (W), Serine (S), Threonine (T) or Alanine (A);
   X₃ is Leucine (L) or Isoleucine (I);
   X₄ is Lysine (K), or Arginine (R), or is omitted;
   X₅ is Glutamine (Q), Arginine (R), Alanine (A) or Lysine (K), or omitted;
   X₆ is Glycine (G), Serine (S), Tyrosine (Y), Cysteine (C), Leucine (L) or Threonine (T);
   X₇ is any amino acid, such as Phenylalanine (F) or Serine (S);
   X₈ is Glycine (G), or is omitted;
   X₉ is Asparagine (N), Histidine (H), Aspartic acid (D) or Threonine (T);
   X₁₀ is Asparagine (N), Serine (S), Threonine (T), Glutamine (Q), Lysine (K) or Glycine (G);
   X₁₁ is Lysine (K), Glutamine (Q), Asparagine (N) or Leucine (L);
   X₁₂ is Tyrosine (Y), Leucine (L) or Phenylalanine (F);
   X₁₃ is Valine (V), Leucine (L) or Alanine (A);
   X₁₄ is Serine (S), Threonine (T), Cysteine (C) or Asparagine (N);
   X₁₅ is Alanine (A), Serine (S) or Glycine (G);
   X₁₆ is Glutamic acid (E), Lysine (K), Arginine (R), Glutamine (Q) or Aspartic acid (D);
   X₁₇ is Asparagine (N), Glycine (G), Glutamine (Q) or Proline (P);
   X₁₈ is Glycine (G), Asparagine (N), Threonine (T) or Aspartic acid (D);
   X₁₉ is Leucine (L) or is omitted;
   X₂₀ is Glycine (G) or Alanine (A), or is omitted;
   X₂₁ is Alanine (A) or Asparagine (N), or is omitted;
   X₂₂ is any amino acid, such as Threonine (T), or is omitted;
   X₂₃ is any amino acid, such as Glycine (G);
   X₂₄ is Proline (P), Alanine (A), Glutamine (Q) or Arginine (R);
   X₂₅ is Leucine (L), Methionine (M), Alanine (A) or Isoleucine (I);
   X₂₆ is any amino acid, such as Threonine (T);
   X₂₇ is Alanine (A), Cysteine (C) or Tryptophan (W);
   X₂₈ is Asparagine (N), Aspartic acid (D), Glutamic acid (E) or Arginine (R);
   X₂₉ is Arginine (R), Alanine (A) or Serine (S);
   X₃₀ is Threonine (T), Aspartic acid (D), Proline (P), Isoleucine (I), Asparagine (N) or Alanine (A);
   X₃₁ is Threonine (T), Alanine (A), Valine (V), Glycine (G), Lysine (K) or Isoleucine (I);
   X₃₂ is Alanine (A), Valine (V), Proline (P) or Leucine (L);
   X₃₃ is Glycine (G), Glutamine (Q), Serine (S) or Aspartic acid (D);
   X₃₄ is any amino acid, such as Glycine (G);
   X₃₇ is any amino acid, such as Glutamine (Q) or Lysine (K);
   or a functional variant thereof wherein the amino acid sequence of said variant differs from SEQ ID NO:220 in that the amino acid sequence of the variant comprises 1 to 6 individual amino acid substitutions,
   b. a repeat region 2 (R2), comprising or consisting of an amino acid sequence SEQ ID NO:221:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁WEX₃₄F,
   wherein
   X₁ is Isoleucine (I) or Valine (V);
   X₂ is Alanine (A), Asparagine (N), Tyrosine (Y) or Threonine (T);
   X₃ is Leucine (L) or Isoleucine (I);
   X₄ is Glutamine (Q), Arginine (R), Leucine (L) or Lysine (K);
   X₅ is Glycine (G), Alanine (A), Asparagine (N) or Serine (S);
   X₆ is any amino acid, such as Alanine (A), or is omitted;
   X₇ is Glycine (G) or Serine (S), or is omitted;
   X₈ is Asparagine (N), Methionine (M), Glutamine (Q), Histidine (H) or Serine (S);
   X₉ is Glycine (G) or Alanine (A);
   X₁₀ is Lysine (K), Leucine (L), Methionine (M), Asparagine (N) or Serine (S);
   X₁₁ is Tyrosine (Y);
   X₁₂ is Valine (V) or Phenylalanine (F);
   X₁₃ is Serine (S), Cysteine (C), Lysine (K) or Glutamine (Q);
   X₁₄ is Alanine (A), Valine (V) or Serine (S);
   X₁₅ is Glutamic acid (E), Aspartic acid (D), Glutamine (Q) or Glycine (G);
   X₁₆ is Asparagine (N), Proline (P), Glycine (G), Threonine (T) or Aspartic acid (D);
   X₁₇ is Glycine (G), Aspartic Acid (D), Isoleucine (I) or Asparagine (N), or is omitted;
   X₁₈ is Valine (V) or Glycine (G), or is omitted;
   X₁₉ is Glycine (G), Threonine (T), Leucine (L), Alanine (A), or Glutamic acid (E) or is omitted;
   X₂₀ is any amino acid, such as Glutamine (Q), or omitted;
   X₂₁ is Proline (P), Alanine (A), Glutamine (Q) or Asparagine (N);
   X₂₂ is Leucine (L), Isoleucine (I), Methionine (M) or Valine (V);
   X₂₃ is Threonine (T), Asparagine (N), Phenylalanine (F) or Arginine (R);
   X₂₄ is Alanine (A), Cysteine (C) or Arginine (R);
   X₂₅ is Asparagine (N), Alanine (A), Threonine (T) or Glutamine (Q);
   X₂₆ is Alanine (A), Glycine (G) or Arginine (R);
   X₂₇ is Threonine (T), Alanine (A), Lysine (K), Proline (P) or Leucine (L);
   X₂₈ is any amino acid, such as Alanine (A);
   X₂₉ is Isoleucine (I), Valine (V), Proline (P), Alanine (A), Lysine (K), Tyrosine (Y) or Leucine (L);
   X₃₀ is Glycine (G), Alanine (A), Glutamic acid (E), Threonine (T), Serine (S), Glutamine (Q) or Aspartic acid (D);
   X₃₁ is Glycine (G), Alanine (A), Aspartic Acid (D), Serine (S), Proline (P) or Lysine (K);
   X₃₄ is any amino acid, such as Lysine (K);
   or a functional variant thereof wherein the amino acid sequence of said variant differs from SEQ ID NO:221 in that the amino acid sequence of the variant comprises 1 to 6 individual amino acid substitutions,
   c. a repeat region 3 (R3), comprising or consisting of an amino acid sequence SEQ ID NO:222:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁X₃₂X₃X₃₄X₃₅WEX₃₈F,
   wherein
   X₁ is Valine (V), Isoleucine (I), Tyrosine (Y) or Phenylalanine (F);
   X₂ is Alanine (A), Serine (S), Cysteine (C), Threonine (T) or Glycine (G);
   X₃ is Leucine (L), Phenylalanine (F) or Isoleucine (I);
   X₄ is Arginine (R), Lysine (K) or Glutamine (Q),
   X₅ is Glycine (G), Alanine (A) or Serine (S);
   X₆ is Arginine (R) or Valine (V), or is omitted;
   X₇ is any amino acid, such as Asparagine (N);
   X₈ is Asparagine (N), Histidine (H), Alanine (A), Lysine (K), or omitted;
   X₉ is Glycine (G), Tryptophan (W) or Asparagine (N);
   X₁₀ is Lysine (K), Leucine (L), Methionine (M), Serine (S), Alanine (A) or Glutamine (Q);
   X₁₁ is Tyrosine (Y), Tryptophan (W) or Phenylalanine (F);
   X₁₂ is Valine (V), Leucine (L), Methionine (M) or Isoleucine (I);
   X₁₃ is Serine (S) or Glutamine (Q);
   X₁₄ is Alanine (A), Serine (S), Histidine (H) or Glycine (G);
   X₁₅ is Glutamic acid (E), Aspartic acid (D), Glutamine (Q), Serine (S) or Asparagine (N);
   X₁₆ is Asparagine (N), Leucine (L), Tryptophan (W), Methionine (M) or Aspartic acid (D);
   X₁₇ is Glycine (G), Serine (S) or Aspartic Acid (D);
   X₁₈ is any amino acid, such as Serine (S) or Threonine (T);
   X₁₉ is Asparagine (N), Valine (V) or Aspartic acid (D), or is omitted;
   X₂₀ is Alanine (A), or is omitted;
   X₂₁ is Serine (S), or is omitted;
   X₂₂ is any amino acid, such as Alanine (A), or is omitted;
   X₂₃ is Proline (P), Alanine (A), Arginine (R), Threonine (T) or Glycine (G);
   X₂₄ is Leucine (L), Methionine (M), Valine (V) or Isoleucine (I);
   X₂₅ is Threonine (T), Tyrosine (Y), Asparagine (N), Leucine (L) or Glutamine (Q);
   X₂₆ is Alanine (A), Cysteine (C), Phenylalanine (F) or Tryptophan (W);
   X₂₇ is Asparagine (N), Alanine (A), Threonine (T) or Serine (S);
   X₂₈ is Arginine (R) or Lysine (K);
   X₂₉ is Aspartic acid (D), or is omitted;
   X₃₀ is Threonine (T), Alanine (A), Proline (P), Serine (S) or Glycine (G);
   X₃₁ is any amino acid, such as Alanine (A);
   X₃₂ is Isoleucine (I), Glutamic acid (E), Proline (P), Alanine (A) or Valine (V);
   X₃₃ is Glycine (G), Lysine (K) or Aspartic acid (D), or is omitted;
   X₃₄ is Glycine (G), Serine (S), Lysine (K), Aspartic acid (D) or Glutamine (Q);
   X₃₅ is Glycine (G), Alanine (A), Aspartic acid (D) or Cysteine (C);
   X₃₈ is any amino acid, such as Lysine (K);
   or a functional variant thereof wherein the amino acid sequence of said variant differs from SEQ ID NO:222 in that the amino acid sequence of the variant comprises 1 to 6 individual amino acid substitutions; and
a polysaccharide comprising one or more β-1,3-glucan units with β-1,6-linked glucose decorations.

In an embodiment of the present disclosure, the hydrogel comprises:
a polypeptide comprising at least two repeat regions selected from the group consisting of:
   a. a repeat region 1 (R1), comprising or consisting of an amino acid sequence

| SEQ ID NO: 8: | | | | | | | | | | | | | | | | | | | | |
|---|---|---|---|---|---|---|---|---|---|---|---|---|---|---|---|---|---|---|---|---|
| I | W | L | - | Q | G | F | - | N | N | K | Y | V | N | S | K | N | G | - | - | - |
| 1 | 2 | 3 | 4 | 5 | 6 | 7 | 8 | 9 | 10 | 11 | 12 | 13 | 14 | 15 | 16 | 17 | 18 | 19 | 20 | 21 |
| Q | G | A | M | W | C | D | S | D | A | P | Q | A | W | E | L | F | | | | |
| 22 | 23 | 24 | 25 | 26 | 27 | 28 | 29 | 30 | 31 | 32 | 33 | 34 | 35 | 36 | 37 | 38 | | | | |

or a functional variant thereof wherein the amino acid sequence of said variant differs from SEQ ID NO:8 in that the amino acid sequence of the variant comprises 1 to 6 individual amino acid substitutions,
b. a repeat region 2 (R2), comprising or consisting of an amino acid sequence

| SEQ ID NO: 10: | | | | | | | | | | | | | | | | | | | | |
|---|---|---|---|---|---|---|---|---|---|---|---|---|---|---|---|---|---|---|---|---|
| I | A | L | R | G | N | - | N | G | M | Y | V | S | S | E | N | G | - | E | Q | A |
| 1 | 2 | 3 | 4 | 5 | 6 | 7 | 8 | 9 | 10 | 11 | 12 | 13 | 14 | 15 | 16 | 17 | 18 | 19 | 20 | 21 |
| I | T | C | N | R | P | A | I | Q | G | W | E | A | F | | | | | | | |
| 22 | 23 | 24 | 25 | 26 | 27 | 28 | 29 | 30 | 31 | 32 | 33 | 34 | 35 | | | | | | | |

or a functional variant thereof wherein the amino acid sequence of said variant differs from SEQ ID NO: 10 in that the amino acid sequence of the variant comprises 1 to 6 individual amino acid substitutions,
c. a repeat region 3 (R3), comprising or consisting of an amino acid sequence

| SEQ ID NO: 12: | | | | | | | | | | | | | | | | | | | | |
|---|---|---|---|---|---|---|---|---|---|---|---|---|---|---|---|---|---|---|---|---|
| V | S | L | R | G | - | S | N | G | L | F | I | S | S | E | N | G | A | - | - | - |
| 1 | 2 | 3 | 4 | 5 | 6 | 7 | 8 | 9 | 10 | 11 | 12 | 13 | 14 | 15 | 16 | 17 | 18 | 19 | 20 | 21 |
| A | A | M | T | C | T | R | - | P | T | A | - | S | G | W | E | A | F | | | |
| 22 | 23 | 24 | 25 | 26 | 27 | 28 | 29 | 30 | 31 | 32 | 33 | 34 | 35 | 36 | 37 | 38 | 39 | | | |

b. or a functional variant thereof wherein the amino acid sequence of said variant differs from SEQ ID NO: 12 in that the amino acid sequence of the variant comprises 1 to 6 individual amino acid substitutions, and
a polysaccharide comprising one or more β-1,3-glucan units with β-1,6-linked glucose decorations.

In an embodiment of the present disclosure, the hydrogel comprises:
a polypeptide comprising at least two repeat regions selected from the group consisting of:
   a. a repeat region 1 (R1), comprising or consisting of an amino acid sequence SEQ ID NO:220:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁X₃₂X₃₃X₃₄WEX₃₇F,
   wherein
   X₁ is Isoleucine (I) or Valine (V);
   X₂ is any amino acid, such as Tryptophan (W), Serine (S), Threonine (T) or Alanine (A);
   X₃ is Leucine (L) or Isoleucine (I);
   X₄ is Lysine (K), or Arginine (R), or is omitted;
   X₅ is Glutamine (Q), Arginine (R), Alanine (A) or Lysine (K), or omitted;
   X₆ is Glycine (G), Serine (S), Tyrosine (Y), Cysteine (C), Leucine (L) or Threonine (T);
   X₇ is any amino acid, such as Phenylalanine (F) or Serine (S);
   X₈ is Glycine (G), or is omitted;
   X₉ is Asparagine (N), Histidine (H), Aspartic acid (D) or Threonine (T);
   X₁₀ is Asparagine (N), Serine (S), Threonine (T), Glutamine (Q), Lysine (K) or Glycine (G);
   X₁₁ is Lysine (K), Glutamine (Q), Asparagine (N) or Leucine (L);
   X₁₂ is Tyrosine (Y), Leucine (L) or Phenylalanine (F);
   X₁₃ is Valine (V), Leucine (L) or Alanine (A);
   X₁₄ is Serine (S), Threonine (T), Cysteine (C) or Asparagine (N);
   X₁₅ is Alanine (A), Serine (S) or Glycine (G);
   X₁₆ is Glutamic acid (E), Lysine (K), Arginine (R), Glutamine (Q) or Aspartic acid (D);
   X₁₇ is Asparagine (N), Glycine (G), Glutamine (Q) or Proline (P);
   X₁₈ is Glycine (G), Asparagine (N), Threonine (T) or Aspartic acid (D);
   X₁₉ is Leucine (L) or is omitted;
   X₂₀ is Glycine (G) or Alanine (A), or is omitted;
   X₂₁ is Alanine (A) or Asparagine (N), or is omitted;
   X₂₂ is any amino acid, such as Threonine (T), or is omitted;
   X₂₃ is any amino acid, such as Glycine (G);
   X₂₄ is Proline (P), Alanine (A), Glutamine (Q) or Arginine (R);
   X₂₅ is Leucine (L), Methionine (M), Alanine (A) or Isoleucine (I);
   X₂₆ is any amino acid, such as Threonine (T);
   X₂₇ is Alanine (A), Cysteine (C) or Tryptophan (W);
   X₂₈ is Asparagine (N), Aspartic acid (D), Glutamic acid (E) or Arginine (R);
   X₂₉ is Arginine (R), Alanine (A) or Serine (S);
   X₃₀ is Threonine (T), Aspartic acid (D), Proline (P), Isoleucine (I), Asparagine (N) or Alanine (A);
   X₃₁ is Threonine (T), Alanine (A), Valine (V), Glycine (G), Lysine (K) or Isoleucine (I);
   X₃₂ is Alanine (A), Valine (V), Proline (P) or Leucine (L);
   X₃₃ is Glycine (G), Glutamine (Q), Serine (S) or Aspartic acid (D);
   X₃₄ is any amino acid, such as Glycine (G);
   X₃₇ is any amino acid, such as Glutamine (Q) or Lysine (K);
   or a functional variant thereof wherein the amino acid sequence of said variant differs from SEQ ID NO:220 in that the amino acid sequence of the variant comprises 1 to 6 individual amino acid substitutions,
   b. a repeat region 2 (R2), comprising or consisting of an amino acid sequence SEQ ID NO:221:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁WEX₃₄F,
   wherein
   X₁ is Isoleucine (I) or Valine (V);
   X₂ is Alanine (A), Asparagine (N), Tyrosine (Y) or Threonine (T);
   X₃ is Leucine (L) or Isoleucine (I);
   X₄ is Glutamine (Q), Arginine (R), Leucine (L) or Lysine (K);
   X₅ is Glycine (G), Alanine (A), Asparagine (N) or Serine (S);
   X₆ is any amino acid, such as Alanine (A), or is omitted;
   X₇ is Glycine (G) or Serine (S), or is omitted;
   X₈ is Asparagine (N), Methionine (M), Glutamine (Q), Histidine (H) or Serine (S);
   X₉ is Glycine (G) or Alanine (A);
   X₁₀ is Lysine (K), Leucine (L), Methionine (M), Asparagine (N) or Serine (S);
   X₁₁ is Tyrosine (Y);
   X₁₂ is Valine (V) or Phenylalanine (F);
   X₁₃ is Serine (S), Cysteine (C), Lysine (K) or Glutamine (Q);
   X₁₄ is Alanine (A), Valine (V) or Serine (S);
   X₁₅ is Glutamic acid (E), Aspartic acid (D), Glutamine (Q) or Glycine (G);
   X₁₆ is Asparagine (N), Proline (P), Glycine (G), Threonine (T) or Aspartic acid (D);
   X₁₇ is Glycine (G), Aspartic Acid (D), Isoleucine (I) or Asparagine (N), or is omitted;
   X₁₈ is Valine (V) or Glycine (G), or is omitted;
   X₁₉ is Glycine (G), Threonine (T), Leucine (L), Alanine (A), or Glutamic acid (E) or is omitted;
   X₂₀ is any amino acid, such as Glutamine (Q), or omitted;
   X₂₁ is Proline (P), Alanine (A), Glutamine (Q) or Asparagine (N);
   X₂₂ is Leucine (L), Isoleucine (I), Methionine (M) or Valine (V);
   X₂₃ is Threonine (T), Asparagine (N), Phenylalanine (F) or Arginine (R);
   X₂₄ is Alanine (A), Cysteine (C) or Arginine (R);
   X₂₅ is Asparagine (N), Alanine (A), Threonine (T) or Glutamine (Q);
   X₂₆ is Alanine (A), Glycine (G) or Arginine (R);
   X₂₇ is Threonine (T), Alanine (A), Lysine (K), Proline (P) or Leucine (L);
   X₂₈ is any amino acid, such as Alanine (A);
   X₂₉ is Isoleucine (I), Valine (V), Proline (P), Alanine (A), Lysine (K), Tyrosine (Y) or Leucine (L);
   X₃₀ is Glycine (G), Alanine (A), Glutamic acid (E), Threonine (T), Serine (S), Glutamine (Q) or Aspartic acid (D);
   X₃₁ is Glycine (G), Alanine (A), Aspartic Acid (D), Serine (S), Proline (P) or Lysine (K);
   X₃₄ is any amino acid, such as Lysine (K);
   or a functional variant thereof wherein the amino acid sequence of said variant differs from SEQ ID NO:221 in that the amino acid sequence of the variant comprises 1 to 6 individual amino acid substitutions,
   c. a repeat region 3 (R3), comprising or consisting of an amino acid sequence SEQ ID NO:222:
   X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉ X₃₀X₃₁X₃₂X₃X₃₄X₃₅WEX₃₈F,
   wherein
   X₁ is Valine (V), Isoleucine (I), Tyrosine (Y) or Phenylalanine (F);
   X₂ is Alanine (A), Serine (S), Cysteine (C), Threonine (T) or Glycine (G);
   X₃ is Leucine (L), Phenylalanine (F) or Isoleucine (I);
   X₄ is Arginine (R), Lysine (K) or Glutamine (Q),
   X₅ is Glycine (G), Alanine (A) or Serine (S);
   X₆ is Arginine (R) or Valine (V), or is omitted;
   X₇ is any amino acid, such as Asparagine (N);
   X₈ is Asparagine (N), Histidine (H), Alanine (A), Lysine (K), or omitted;
   X₉ is Glycine (G), Tryptophan (W) or Asparagine (N);
   X₁₀ is Lysine (K), Leucine (L), Methionine (M), Serine (S), Alanine (A) or Glutamine (Q);
   X₁₁ is Tyrosine (Y), Tryptophan (W) or Phenylalanine (F);
   X₁₂ is Valine (V), Leucine (L), Methionine (M) or Isoleucine (I);
   X₁₃ is Serine (S) or Glutamine (Q);
   X₁₄ is Alanine (A), Serine (S), Histidine (H) or Glycine (G);
   X₁₅ is Glutamic acid (E), Aspartic acid (D), Glutamine (Q), Serine (S) or Asparagine (N);
   X₁₆ is Asparagine (N), Leucine (L), Tryptophan (W), Methionine (M) or Aspartic acid (D);
   X₁₇ is Glycine (G), Serine (S) or Aspartic Acid (D);
   X₁₈ is any amino acid, such as Serine (S) or Threonine (T);
   X₁₉ is Asparagine (N), Valine (V) or Aspartic acid (D), or is omitted;
   X₂₀ is Alanine (A), or is omitted;
   X₂₁ is Serine (S), or is omitted;
   X₂₂ is any amino acid, such as Alanine (A), or is omitted;
   X₂₃ is Proline (P), Alanine (A), Arginine (R), Threonine (T) or Glycine (G);
   X₂₄ is Leucine (L), Methionine (M), Valine (V) or Isoleucine (I);
   X₂₅ is Threonine (T), Tyrosine (Y), Asparagine (N), Leucine (L) or Glutamine (Q);
   X₂₆ is Alanine (A), Cysteine (C), Phenylalanine (F) or Tryptophan (W);
   X₂₇ is Asparagine (N), Alanine (A), Threonine (T) or Serine (S);
   X₂₈ is Arginine (R) or Lysine (K);
   X₂₉ is Aspartic acid (D), or is omitted;
   X₃₀ is Threonine (T), Alanine (A), Proline (P), Serine (S) or Glycine (G);
   X₃₁ is any amino acid, such as Alanine (A);
   X₃₂ is Isoleucine (I), Glutamic acid (E), Proline (P), Alanine (A) or Valine (V);
   X₃₃ is Glycine (G), Lysine (K) or Aspartic acid (D), or is omitted;
   X₃₄ is Glycine (G), Serine (S), Lysine (K), Aspartic acid (D) or Glutamine (Q);
   X₃₅ is Glycine (G), Alanine (A), Aspartic acid (D) or Cysteine (C);
   X₃₈ is any amino acid, such as Lysine (K);
   or a functional variant thereof wherein the amino acid sequence of said variant differs from SEQ ID NO:222 in that the amino acid sequence of the variant comprises 1 to 6 individual amino acid substitutions; and the polysaccharide Scleroglucan.

In an embodiment of the present disclosure, the hydrogel comprises:
a polypeptide comprising at least two repeat regions selected from the group consisting of:
   a. a repeat region 1 (R1), comprising or consisting of an amino acid sequence

| SEQ ID NO: 8: | | | | | | | | | | | | | | | | | | | | |
|---|---|---|---|---|---|---|---|---|---|---|---|---|---|---|---|---|---|---|---|---|
| I | W | L | - | Q | G | F | - | N | N | K | Y | V | N | S | K | N | G | - | - | - |
| 1 | 2 | 3 | 4 | 5 | 6 | 7 | 8 | 9 | 10 | 11 | 12 | 13 | 14 | 15 | 16 | 17 | 18 | 19 | 20 | 21 |
| Q | G | A | M | W | C | D | S | D | A | P | Q | A | W | E | L | F | | | | |
| 22 | 23 | 24 | 25 | 26 | 27 | 28 | 29 | 30 | 31 | 32 | 33 | 34 | 35 | 36 | 37 | 38 | | | | |

or a functional variant thereof wherein the amino acid sequence of said variant differs from SEQ ID NO:8 in that the amino acid sequence of the variant comprises 1 to 6 individual amino acid substitutions,
b. a repeat region 2 (R2), comprising or consisting of an amino acid sequence

| SEQ ID NO: 10: | | | | | | | | | | | | | | | | | | | | |
|---|---|---|---|---|---|---|---|---|---|---|---|---|---|---|---|---|---|---|---|---|
| I | A | L | R | G | N | - | N | G | M | Y | V | S | S | E | N | G | - | E | Q | A |
| 1 | 2 | 3 | 4 | 5 | 6 | 7 | 8 | 9 | 10 | 11 | 12 | 13 | 14 | 15 | 16 | 17 | 18 | 19 | 20 | 21 |
| I | T | C | N | R | P | A | I | Q | G | W | E | A | F | | | | | | | |
| 22 | 23 | 24 | 25 | 26 | 27 | 28 | 29 | 30 | 31 | 32 | 33 | 34 | 35 | | | | | | | |

or a functional variant thereof wherein the amino acid sequence of said variant differs from SEQ ID NO: 10 in that the amino acid sequence of the variant comprises 1 to 6 individual amino acid substitutions,
c. a repeat region 3 (R3), comprising or consisting of an amino acid sequence

| SEQ ID NO: 12: | | | | | | | | | | | | | | | | | | | | |
|---|---|---|---|---|---|---|---|---|---|---|---|---|---|---|---|---|---|---|---|---|
| V | S | L | R | G | - | S | N | G | L | F | I | S | S | E | N | G | A | - | - | - |
| 1 | 2 | 3 | 4 | 5 | 6 | 7 | 8 | 9 | 10 | 11 | 12 | 13 | 14 | 15 | 16 | 17 | 18 | 19 | 20 | 21 |
| A | A | M | T | C | T | R | - | P | T | A | - | S | G | W | E | A | F | | | |
| 22 | 23 | 24 | 25 | 26 | 27 | 28 | 29 | 30 | 31 | 32 | 33 | 34 | 35 | 36 | 37 | 38 | 39 | | | |

b. or a functional variant thereof wherein the amino acid sequence of said variant differs from SEQ ID NO: 12 in that the amino acid sequence of the variant comprises 1 to 6 individual amino acid substitutions, and
the polysaccharide scleroglucan.

In an embodiment of the present disclosure, the hydrogel comprises:
a polypeptide comprising an amino acid sequence having at least 75% sequence identity to SEQ ID NO:3 or SEQ ID NO:4, SEQ ID NO:20 to SEQ ID NO:33, or SEQ ID NO: 71 to 219, and capable of binding one or more saccharide units, and
a polysaccharide comprising one or more β-1,3-glucan units with β-1,6-linked glucose decorations.

In an embodiment of the present disclosure, the hydrogel comprises:
a polypeptide comprising an amino acid sequence having at least 75% sequence identity to SEQ ID NO:3 or SEQ ID NO:4, SEQ ID NO:20 to SEQ ID NO:33, or SEQ ID NO: 71 to 219, and capable of binding one or more saccharide units, and
the polysaccharide scleroglucan.

In an embodiment of the present disclosure, the hydrogel comprises:
a polypeptide comprising an amino acid sequence having at least 75% sequence identity to SEQ ID NO:3 or SEQ ID NO:4, or SEQ ID NO:20 to SEQ ID NO:33, and capable of binding one or more saccharide units, and
a polysaccharide comprising one or more β-1,3-glucan units with β-1,6-linked glucose decorations.

In an embodiment of the present disclosure, the hydrogel comprises:
a polypeptide comprising an amino acid sequence having at least 75% sequence identity to SEQ ID NO:3 or SEQ ID NO:4, or SEQ ID NO:20 to SEQ ID NO:33, and capable of binding one or more saccharide units, and
the polysaccharide scleroglucan.

In another aspect the present disclosure relates to a cross-linker for crosslinking polysaccharides to form hydrogels, wherein said cross-linker is a polypeptide as described herein.

In yet another aspect the present disclosure relates to a method of manufacturing a hydrogel, said method comprising:
a. providing a solution comprising a polysaccharide,
b. providing a solution comprising a polypeptide as described herein.
c. mixing the solutions under continuous stirring at room temperature,
thereby obtaining a hydrogel via cross-linking of the polysaccharide.

In an embodiment of the present disclosure, the hydrogel comprises a polysaccharide as described herein.

### EXAMPLES

### Example 1. Protein Domains Identified as Part of the CBM Family

Database searches for CBM domain homologues were carried out using BLASTP with C. pinensis Cp-F1 protein as query against the non-redundant protein sequences dataset of the Genbank database at the National Center for Biotechnology Information (NCBI) (http://www.ncbi.nlm.nih.gov). Sequences containing homologues to Cp-F1 were selected to generate a CBM containing-protein subset for further analysis. This subset was evaluated using the taxonomy browser at NCBI (http://www.ncbi.nlm.nih.gov/Taxonomy). Incomplete and redundant entries were removed. Additionally, only one exemplary species was selected from each genus, and the final dataset contained 164 sequences.

All identified carbohydrate binding protein modules are summarized in Table 1. The table contains gene accession numbers, species name, abbreviated identifier, and sequence identity number refereeing to the ST.26 sequence listing enclosed, for all carbohydrate binding protein modules identified. The identifiers comprise an abbreviation of the species name and the number of amino acids in the full-length multi-modular protein that contains one of the carbohydrate binding domains. In native form, these carbohydrate binding domains are all part of multi-modular proteins with carbohydrate-degrading functions.

TABLE 1
Protein domains identified as part of the CBM92 family

| | | NCBI accession | |
|---|---|---|---|
| Identifier | Species | No. | Comments |
| Aa-F1 | Aquimarina aggregata | WP_066313671.1 | SEQ ID NO: 22 |
| Aa-F2 | Aquimarina aggregata | WP_066320065.1 | SEQ ID NO: 23 |
| Aag394 | Aquimarina aggregata | WP_066313446.1 | SEQ ID NO: 171 |
| Aag521 | Aquimarina aggregata | WP_066310828.1 | SEQ ID NO: 205 |
| Aag531 | Aquimarina aggregata | WP_157766178.1 | SEQ ID NO: 82 |
| Aag641 | Aquimarina aggregata | WP_066315015.1 | SEQ ID NO: 203 |
| Aaq414 | Algibacter aquaticus | WP_069828882.1 | SEQ ID NO: 99 |
| Aaq586 | Algibacter aquaticus | WP_069829724.1 | SEQ ID NO: 170 |
| Aba478 | Armatimonadetes bacterium | NMC82449.1 | SEQ ID NO: 155 |
| Aba500 | Actinobacteria bacterium | TMM36148.1 | SEQ ID NO: 218 |
| Aba509 | Acidimicrobiia bacterium | MBA3268160.1 | SEQ ID NO: 219 |
| Aba692 | Actinobacteria bacterium | TMM30772.1 | SEQ ID NO: 216 |
| Ag1548 | Actinoplanes globisporus | WP_020509689.1 | SEQ ID NO: 144 |
| Apo527 | Apiotrichum porosum | XP_028473893.1 | SEQ ID NO: 138 |
| Aro542 | Actinospica robiniae | WP_063627936.1 | SEQ ID NO: 194 |
| Ase531 | Anaeromicrobium sediminis | WP_095133838.1 | SEQ ID NO: 146 |
| Bba399 | Blastocatellia bacterium | MBO0723753.1 | SEQ ID NO: 71 |
| Bba444 | Blastocatellia bacterium | MBL8206846.1 | SEQ ID NO: 180 |
| Bba510 | Blastocatellia bacterium | PWT82357.1 | SEQ ID NO: 135 |
| Bba710 | Blastocatellia bacterium | MBL8206846.1 | SEQ ID NO: 147 |
| Bgl556 | Burkholderia gladioli | WP_186190248.1 | SEQ ID NO: 140 |
| Bso634 | Bacillus solimangrovi | OEH91538.1 | SEQ ID NO: 187 |
| Cak132 | Coraliomargarita akajimensis DSM | ADE54029.1 | SEQ ID NO: 197 |
| | 45221 | | |
| Cak396 | Coraliomargarita akajimensis DSM | ADE54011.1 | SEQ ID NO: 195 |
| | 45221 | | |
| Cak417 | Coraliomargarita akajimensis DSM | ADE53100.1 | SEQ ID NO: 104 |
| | 45221 | | |
| Cak440 | Coraliomargarita akajimensis DSM | ADE53121.1 | SEQ ID NO: 196 |
| | 45221 | | |
| Cco628 | Capsulimonas corticalis | WP_119324998.1 | SEQ ID NO: 177 |
| Cfa651 | Coleofasciculus sp. FACHB-129 | WP_190676778.1 | SEQ ID NO: 148 |
| Cfl312 | Chryseolinea flava | WP_112749069.1 | SEQ ID NO: 116 |
| Cfl474 | Chryseolinea flava | WP_112748658.1 | SEQ ID NO: 113 |
| Cfl530 | Chryseolinea flava | WP_112748659.1 | SEQ ID NO: 119 |
| Cfl723 | Chryseolinea flava | RAW00633.1 | SEQ ID NO: 128 |
| Cfl728 | Chryseolinea flava | WP_185147044.1 | SEQ ID NO: 114 |
| Cfo322 | Coptotermes formosanus | AEW67353.1 | SEQ ID NO: 153 |
| Cfu445 | Cystobacter fuscus | WP_095989217.1 | SEQ ID NO: 78 |
| Ckc439 | Comamonas sp. KCTC 72670 | WP_191267545.1 | SEQ ID NO: 73 |
| CPa717 | Candidatus Poseidoniales | RCH73252.1 | SEQ ID NO: 169 |
| | archaeon | | |
| CPa752 | Candidatus Poseidoniales | DAC55731.1 | SEQ ID NO: 157 |
| | archaeon | | |
| CPa871 | Candidatus Poseidoniales | DAC30974.1 | SEQ ID NO: 164 |
| | archaeon | | |
| CPa891 | Candidatus Poseidoniales | DAC10775.1 | SEQ ID NO: 156 |
| | archaeon | | |
| Cp-F1 | Chitinophaga pinensis DSM 2588 | ACU60063.1 | SEQ ID NO: 3; |
| | | | SEQ ID NO: 20 |
| | | | (without the N- |
| | | | and C-terminal |
| | | | tags) |
| Cp-F2 | Chitinophaga pinensis DSM 2588 | ACU60063.1 | SEQ ID |
| | | | NO: 4;SEQ ID |
| | | | NO: 21 (without |
| | | | the C-terminal |
| | | | tags) |
| Cp-F3 | Chitinophaga pinensis DSM 2588 | ACU61803.1 | SEQ ID NO: 24 |
| Cp-F4 | Chitinophaga pinensis DSM 2588 | ACU61215.1 | SEQ ID NO: 25 |
| Cp-F5 | Chitinophaga pinensis DSM 2588 | ACU62541.1 | SEQ ID NO: 26 |
| Cp-F6 | Chitinophaga pinensis DSM 2588 | ACU61621.1 | SEQ ID NO: 27 |
| Cp-F7 | Chitinophaga pinensis DSM 2588 | ACU61949.1 | SEQ ID NO: 28 |
| Cru540 | Catenulispora rubra | WP_194909551.1 | SEQ ID NO: 193 |
| Cse639 | Chengkuizengella sediminis | WP_162037177.1 | SEQ ID NO: 186 |
| Csi458 | Camellia sinensis | XP_028082931.1 | SEQ ID NO: 201 |
| Cst390 | Cylindrospermum stagnale | WP_172642182.1 | SEQ ID NO: 175 |
| Cte424 | Corallococcus terminator | WP_120540014.1 | SEQ ID NO: 76 |
| Cur554 | Cellulomonas sp. URHD0024 | WP_028051117.1 | SEQ ID NO: 141 |
| Dal637 | Dictyobacter alpinus | WP_126631529.1 | SEQ ID NO: 190 |
| Dko637 | Dictyobacter kobayashii | WP_126557407.1 | SEQ ID NO: 191 |
| Dm-F1 | Draconibacterium sp. GM2-18 | WP_163322777.1 | SEQ ID NO: 70 |
| Dsi395 | Dinghuibacter silviterrae | WP_133997709.1 | SEQ ID NO: 107 |
| Dso399 | Deminuibacter soli | WP_116848117.1 | SEQ ID NO: 108 |
| Dsu946 | Dactylosporangium sucinum | WP_190249133.1 | SEQ ID NO: 183 |
| Dth161 | Dichotomicrobium | WP_119060661.1 | SEQ ID NO: 212 |
| | thermohalophilum | | |
| Dvl638 | Dictyobacter vulcani | WP_151757874.1 | SEQ ID NO: 189 |
| Ea-F1 | Euryarchaeota archaeon | MBS73237.1 | SEQ ID NO: 29 |
| Ear1058 | Euryarchaeota archaeon | MBV23397.1 | SEQ ID NO: 165 |
| Ear1096 | Euryarchaeota archaeon | MBC64308.1 | SEQ ID NO: 167 |
| Ear344 | Euryarchaeota archaeon | MBJ29332.1 | SEQ ID NO: 168 |
| Ear436 | Euryarchaeota archaeon | MBH33816.1 | SEQ ID NO: 161 |
| Ear461 | Euryarchaeota archaeon | MBJ36561.1 | SEQ ID NO: 166 |
| Ear692 | Euryarchaeota archaeon | MAN07855.1 | SEQ ID NO: 154 |
| Ear751 | Euryarchaeota archaeon | MAY05287.1 | SEQ ID NO: 158 |
| Ear818 | Euryarchaeota archaeon | MBP65661.1 | SEQ ID NO: 160 |
| Ear837 | Euryarchaeota archaeon | MBM54141.1 | SEQ ID NO: 159 |
| Ear871 | Euryarchaeota archaeon | MBA86910.1 | SEQ ID NO: 163 |
| Ear893 | Euryarchaeota archaeon | RAH12883.1 | SEQ ID NO: 162 |
| Fae792 | Fibrella aestuarina | WP_083891454.1 | SEQ ID NO: 129 |
| Fba372 | Flavobacteriaceae bacterium M625 | TPN87735.1 | SEQ ID NO: 81 |
| Fba837 | Flavobacteriaceae bacterium M625 | TPN83373.1 | SEQ ID NO: 204 |
| Fmu672 | Fischerella muscicola | WP_016861236.1 | SEQ ID NO: 152 |
| Fsu758 | Flavobacterium subsaxonicum | WP_051200144.1 | SEQ ID NO: 90 |
| Gso734 | Geomonas soli | WP_124539163.1 | SEQ ID NO: 210 |
| Hau734 | Herpetosiphon aurantiacus DSM | ABX04162.1 | SEQ ID NO: 207 |
| | 785 | | |
| Hba589 | Hyphomicrobiaceae bacterium | TXH09896.1 | SEQ ID NO: 174 |
| Hbr682 | Hymenobacter sp. BRD128 | WP_173117794.1 | SEQ ID NO: 122 |
| HII733 | Herpetosiphon llansteffanensis | WP_110518824.1 | SEQ ID NO: 206 |
| Hmi438 | Hyalangium minutum | WP_044181817.1 | SEQ ID NO: 75 |
| Hoc627 | Haliangium ochraceum | WP_012831313.1 | SEQ ID NO: 94 |
| Hts386 | Hamadaea tsunoensis | WP_027344156.1 | SEQ ID NO: 178 |
| Hvu295 | Hydra vulgaris | AAZ31366.1 | SEQ ID NO: 213 |
| Kje421 | Kordia jejudonensis | WP_053002293.1 | SEQ ID NO: 172 |
| Kje633 | Kordia jejudonensis | WP_046756685.1 | SEQ ID NO: 91 |
| Kra639 | Ktedonobacter racemifer | WP_007909656.1 | SEQ ID NO: 188 |
| Mae184 | Microcystis aeruginosa | WP_139374347.1 | SEQ ID NO: 93 |
| Mar481 | Marinoscillum sp. 108 | WP_159579538.1 | SEQ ID NO: 111 |
| Mba425 | Myxococcaceae bacterium | RYZ39347.1 | SEQ ID NO: 77 |
| Mdi744 | Mangrovibacterium diazotrophicum | WP_120272090.1 | SEQ ID NO: 87 |
| Mha438 | Myxococcus hansupus | WP_002636775.1 | SEQ ID NO: 72 |
| Mlu521 | Marivirga lumbricoides | PTB97274.1 | SEQ ID NO: 100 |
| Mlu658 | Marivirga lumbricoides | WP_188463823.1 | SEQ ID NO: 98 |
| Mno729 | Mitsuaria noduli | OWQ45395.1 | SEQ ID NO: 209 |
| Mor409 | Micromonospora orduensis | WP_139587827.1 | SEQ ID NO: 214 |
| Mor696 | Micromonospora orduensis | WP_139588083.1 | SEQ ID NO: 215 |
| Mor774 | Micromonospora orduensis | WP_139586123.1 | SEQ ID NO: 181 |
| Mor942 | Micromonospora orduensis | WP_139584258.1 | SEQ ID NO: 184 |
| Mr-F2 | Mucilaginibacter rubeus | WP_129569879.1 | SEQ ID NO: 31 |
| Mru470b | Mucilaginibacter rubeus | WP_112568850.1 | SEQ ID NO: 121 |
| Mru495 | Mucilaginibacter rubeus | QEM12674.1 | SEQ ID NO: 120 |
| Mr-F3 | Mucilaginibacter rubeus | WP_112575541.1 | SEQ ID NO: 32 |
| Mr-F1 | Mucilaginibacter rubeus | QEM13197.1 | SEQ ID NO: 30 |
| Nan544 | Nonomuraea angiospora | MBE1592301.1 | SEQ ID NO: 143 |
| Nko406 | Niastella koreensis | WP_014216967.1 | SEQ ID NO: 109 |
| Nko548 | Niastella koreensis | WP_014216945.1 | SEQ ID NO: 117 |
| Nko628 | Niastella koreensis | WP_014216968.1 | SEQ ID NO: 95 |
| Nko633 | Niastella koreensis | WP_014221059.1 | SEQ ID NO: 115 |
| Nmu509 | Nakamurella multipartita | WP_138180402.1 | SEQ ID NO: 182 |
| Npa240 | Nostoc parmelioides | WP_190572743.1 | SEQ ID NO: 151 |
| Nxi629 | Nonlabens xiamenensis | WP_124979265.1 | SEQ ID NO: 92 |
| Oko475 | Ohtaekwangia koreensis | WP_079685365.1 | SEQ ID NO: 112 |
| Oko535 | Ohtaekwangia koreensis | WP_079685366.1 | SEQ ID NO: 118 |
| Oko784 | Ohtaekwangia koreensis | WP_079690368.1 | SEQ ID NO: 130 |
| Pba630a | Phycisphaerales bacterium | HCD29691.1 | SEQ ID NO: 124 |
| Pba630b | Phycisphaerales bacterium | MAV55595.1 | SEQ ID NO: 123 |
| Pba677 | Phycisphaerae bacterium | MAW42132.1 | SEQ ID NO: 125 |
| Pbb944 | Plantactinospora sp. BB1 | WP_107259782.1 | SEQ ID NO: 185 |
| Pca421b | Pyxidicoccus sp. CA060A | WP_164018937.1 | SEQ ID NO: 74 |
| Pc-F1 | Pyxidicoccus sp. CA060A | WP_164000589.1 | SEQ ID NO: 33 |
| Pdu408 | Pedobacter duraquae | WP_133555761.1 | SEQ ID NO: 110 |
| Pdu613 | Pedobacter duraquae | WP_133557106.1 | SEQ ID NO: 96 |
| Pfl374 | Phytohabitans flavus | WP_173035271.1 | SEQ ID NO: 217 |
| Phi427 | Pseudarcicella hirudinis | SFP22661.1 | SEQ ID NO: 106 |
| Phu411 | Paenibacillus hunanensis | WP_188774785.1 | SEQ ID NO: 145 |
| Ple522 | Paucimonas lemoignei | WP_132259237.1 | SEQ ID NO: 136 |
| Pso390 | Plantactinospora soyae | MBE1491319.1 | SEQ ID NO: 179 |
| Pso818 | Paraflavitalea soli | AXY74800.1 | SEQ ID NO: 131 |
| Px797 | Pseudoflavitalea sp. X16 | WP_167290596.1 | SEQ ID NO: 132 |
| Rba747 | Rickettsiales bacterium | MBR07351.1 | SEQ ID NO: 88 |
| Rba757 | Rickettsiales bacterium | MBR07353.1 | SEQ ID NO: 89 |
| Rba818 | Rickettsiales bacterium | MBR07521.1 | SEQ ID NO: 126 |
| Rde553 | Roseateles depolymerans | WP_058935987.1 | SEQ ID NO: 142 |
| Rde728 | Roseateles depolymerans | ALV08126.1 | SEQ ID NO: 208 |
| Rov720 | Rhizobacter sp. OV335 | WP_073469098.1 | SEQ ID NO: 211 |
| Rsi471 | Rhododendron simsii | KAF7129156.1 | SEQ ID NO: 199 |
| Rve1039 | Reichenbachiella versicolor | WP_109831106.1 | SEQ ID NO: 173 |
| Sau445 | Stigmatella aurantiaca | WP_002612833.1 | SEQ ID NO: 80 |
| Sau480 | Saccharicrinis aurantiacus | WP_068475139.1 | SEQ ID NO: 101 |
| Sau491 | Saccharicrinis aurantiacus | WP_075602374.1 | SEQ ID NO: 103 |
| Sau502 | Saccharicrinis aurantiacus | WP_075603264.1 | SEQ ID NO: 85 |
| Sau606 | Saccharicrinis aurantiacus | WP_075603263.1 | SEQ ID NO: 84 |
| Sba1159 | Syntrophaceae bacterium | NWF54575.1 | SEQ ID NO: 150 |
| Sba777 | Saprospirales bacterium | HAQ39283.1 | SEQ ID NO: 134 |
| Sca510 | Saccharicrinis carchari | WP_142533534.1 | SEQ ID NO: 102 |
| Sf1609 | Sediminitomix flava | WP_109621230.1 | SEQ ID NO: 83 |
| Sin534 | Sesamum indicum | XP_011097508.1 | SEQ ID NO: 200 |
| Spe686 | Sinomicrobium pectinilyticum | WP_123218104.1 | SEQ ID NO: 127 |
| Src742 | Sunxiuqinia sp. RC1_OXG_1F | WP_159523523.1 | SEQ ID NO: 86 |
| Ssi681 | Symploca sp. SIO2E6 | NET60575.1 | SEQ ID NO: 149 |
| Sto533 | Senna tora | KAF7834451.1 | SEQ ID NO: 198 |
| T7g555 | Trinickia sp. 7GSK02 | WP_136892366.1 | SEQ ID NO: 139 |
| Tm425 | Tenacibaculum sp. M341 | WP_132722194.1 | SEQ ID NO: 202 |
| Tm671 | Tenacibaculum sp. M341 | WP_132722652.1 | SEQ ID NO: 176 |
| Tsa488 | Terriglobus saanensis | WP_013567755.1 | SEQ ID NO: 192 |
| Vba1138 | Verrucomicrobia bacterium | NCX47021.1 | SEQ ID NO: 133 |
| Vba185 | Verrucomicrobia bacterium | MBD99715.1 | SEQ ID NO: 105 |
| Vba370 | Verrucomicrobia bacterium | PYK05847.1 | SEQ ID NO: 137 |
| Vgd439 | Vitiosangium sp. GDMCC 1.1324 | WP_108075538.1 | SEQ ID NO: 79 |
| Zoi673 | Zobellia sp. O113 | WP_088696129.1 | SEQ ID NO: 97 |

### Example 2. Recombinant Production of CBM92 Domain Proteins of the Present Disclosure

Certain genes explored in this study were synthesised in a proprietary vector by ThermoFisher GeneArt; these were then sub-cloned into the expression vector pET21a (ThermoFisher), which carries a C-terminal His6-tag and confers ampicillin resistance (marked as “Commercial synthesis” in Table 2). Other genes were cloned in-house from genomic C. pinensis DNA (DSMZ, Germany) (marked as “Cloned from gDNA into PLATE31” in Table 2).

Recombinant proteins were generated according to the cloning strategy described in Table 2. CBM domains were cloned from large multi-modular genes. The range of nucleotides specified therefore refers to the fragment that was cloned from the full-length gene. The size (kDa) refers to the resulting recombinant CBM protein. gDNA=genomic DNA.

TABLE 2
Cloning strategy

| | Genbank | | | |
|---|---|---|---|---|
| | accession no. | | | |
| | and range of | | | |
| | amino acids | | | |
| | included in | Protein | Size | Cloning |
| Organism | construct | name | (kDa) | strategy* |
| Chitinophaga | ACU60063.1 | Cp-F1 | 16.2 | Commercial |
| pinensis | 448-576 | | | synthesis |
| Chitinophaga | ACU60063.1 | Cp-F2 | 16.2 | Commercial |
| pinensis | 888-1012 | Used in | | synthesis |
| | | structural | | |
| | | analysis | | |
| Chitinophaga | ACU60063.1 | Cp-F2 | 15.4 | Cloned from |
| pinensis | 888-1012 | Used in | | gDNA into |
| | | Native- | | pLATE31 |
| | | PAGE | | |
| Chitinophaga | ACU61803.1 | Cp-F3 | 16.0 | Cloned from |
| pinensis | 476-603 | | | gDNA into |
| | | | | pET21a |
| Chitinophaga | ACU61215.1 | Cp-F4 | 14.9 | Cloned from |
| pinensis | 414-542 | | | gDNA into |
| | | | | pLATE31 |
| Chitinophaga | ACU62541.1 | Cp-F5 | 16.5 | Cloned from |
| pinensis | 275-409 | | | gDNA into |
| | | | | pET21a |
| Chitinophaga | ACU61621.1 | Cp-F6 | 15.9 | Cloned from |
| pinensis | 429-552 | | | gDNA into |
| | | | | pET21a |
| Chitinophaga | ACU61949.1 | Cp-F7 | 15.8 | Cloned from |
| pinensis | 788-912 | | | gDNA into |
| | | | | pET21a |
| Draconibacterium | WP_163322777.1 | Dm-F1 | 16.9 | Commercial |
| mangrovi | 534-666 | | | synthesis |
| Aquimarina | WP_066313671.1 | Aa-F1 | 17.2 | Commercial |
| aggregata | 787-919 | | | synthesis |
| Euryarchaeota | MBS73237.1 | Ea-F1 | 17.9 | Commercial |
| archaeon | 119-254 | | | synthesis |
| Aquimarina | WP_066320065.1 | Aa-F2 | 18.2 | Commercial |
| aggregata | 456-596 | | | synthesis |
| Pyxidicoccus | WP_164000589.1 | Pc-F1 | 17.6 | Commercial |
| caerfyrddinensis | 27-170 | | | synthesis |

*Commercial synthesis = gene was synthesised by ThermoFisher GeneArt, then transferred into plasmid pET21a by restriction digestion cloning.

Results: Protein production and purification was analysed by SDS-PAGE every time. Protein yield measured every time by Bradford assay and/or by using a nanodrop. SDS-PAGE analysis confirmed successful production and purification for all constructs (data not shown). Typical protein yield for Cp-F1 is 150 mg pure protein from a 1 L culture of over-expressing E. coli cells. Other proteins were produced and purified with a yield of 25-75 mg and up to 150 mg pure protein from a 1 L culture.

### Example 3. The CBM92 Domain Proteins of the Present Disclosure can Bind the Polysaccharide Scleroglucan

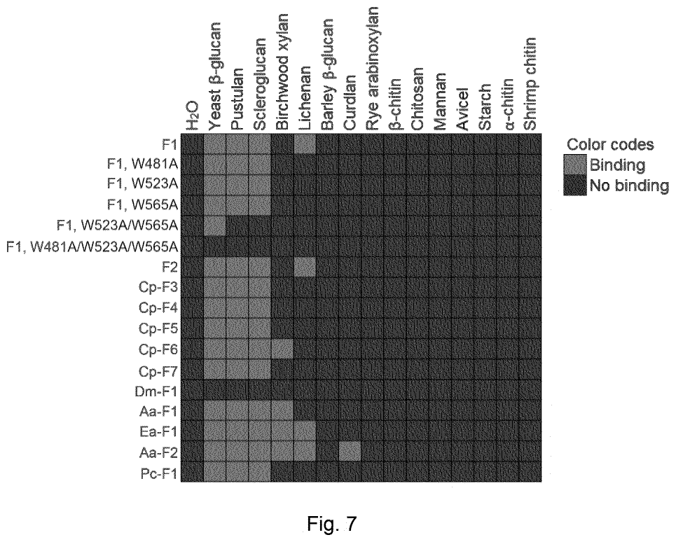

Binding to water-insoluble polysaccharides for 12 proteins identified as part of the CBM92 family (see Table 3) as well as five F1 variants where the Tryptophan (W/Trp) in the binding site “WExF” motif was converted by site-directed mutagenesis to an Alanine (A/Ala), in either one (α, β or γ), two (β/γ) or three (α/β/γ) binding domains was tested by pull-down assay (FIG. 7). Protein and an excess of polysaccharide were incubated together in water using a slowly rotating incubator at room temperature. After 2-3 hours of incubation, samples were centrifuged lightly in order to pellet the insoluble polysaccharides. Any protein that was bound to the polysaccharide was pulled down into this pellet, whereas unbound protein remained in the supernatant. Samples were taken from the supernatant and analysed by SDS-PAGE. A different assay, affinity gel electrophoresis, was used to test binding to Laminarin, which is a highly soluble polysaccharide due to its low molecular weight (see also Tomme et al. 2020).

TABLE 3
Binding of cloned and characterized CBM92
domain proteins to scleroglucan

| | | Binding to |
|---|---|---|
| Protein name | SEQ ID NO: | Scleroglucan |
| Cp-F1 | SEQ ID NO: 3 | + |
| W481A | SEQ ID NO: 5 | + |
| W523A | SEQ ID NO: 6 | + |
| W565A | SEQ ID NO: 7 | + |
| W523A/W565A | SEQ ID NO: 17 | − |
| W481A/W523A/W565A | SEQ ID NO: 19 | − |
| Cp-F2 | SEQ ID NO: 4 | + |
| Cp-F3 | SEQ ID NO: 24 | + |
| Cp-F4 | SEQ ID NO: 25 | + |
| Cp-F5 | SEQ ID NO: 26 | + |
| Cp-F6 | SEQ ID NO: 27 | + |
| Cp-F7 | SEQ ID NO: 28 | + |
| Dm-F1 | SEQ ID NO: 70 | − |
| Aa-F1 | SEQ ID NO: 22 | + |
| Ea-F1 | SEQ ID NO: 29 | + |
| Aa-F2 | SEQ ID NO: 23 | + |
| Pc-F1 | SEQ ID NO: 33 | + |

Results: The absence of a protein band in SDS-PAGE indicated that the protein successfully bound to the polysaccharide (Table 4). A consistent affinity for binding to polysaccharides containing the Glc-β-1,6-Glc linkage, namely pustulan (linear β-1,6-glucan), as well as scleroglucan and yeast β-glucan (both consisting of β-1,3-glucan chains with single substitutions of β-1,6-linked glucosyl residues) was shown. Some of the CBM proteins tested also showed some binding to curdlan, a linear β-1,3-glucan, and in some cases there was binding to lichenan, which comprises β-1,3- and β-1,4-linked glucosyl residues. There was also weak binding to birchwood xylan in some cases, suggesting the capacity for more relaxed specificity in certain CBM proteins. Of note, Dm-F1, which naturally lacks all binding-site Trp residues, did not bind any of the polysaccharides tested (FIG. 7). For all proteins tested, no binding was observed to cellulose, other types of xylan, various types of β-mannan, chitin, starch, chitosan, or mixed linkage β-1,3:1,4-glucan from plants.

All wild type proteins that showed binding with scleroglucan were also confirmed to be gel-forming.

TABLE 4
Binding of the proteins Cp-F1 and Cp-F2 to polysaccharides. A minus symbol (−)
signifies that there was no binding. A plus symbol (+) indicates that there was
binding; increasing numbers of plus symbols indicates comparatively stronger binding.

| | Polysaccharide | Polysaccharide | | |
|---|---|---|---|---|
| Polysaccharide | structure | source | Cp-F1 | Cp-F2 |
| α-chitin | Antiparallel chains | Fungal cell walls, | − | − |
| | of β-1,4-linked | some shellfish | | |
| | GlcNAc | and insects | | |
| β-chitin | Parallel chains of | Some shellfish | − | − |
| | β-1,4-linked | | | |
| | GlcNAc | | | |
| Chitosan | Linear β-1,4-GlcN | De-acetylated | − | − |
| | | form of chitin | | |
| Ivory nut | Linear β-1,4- | Plants | − | − |
| mannan | mannan | | | |
| Barley | Linear mixed- | Plants | − | − |
| β-glucan | linkage β-1,3- and | | | |
| | β-1,4-glucan | | | |
| Pustulan | Linear β-1,6-glucan | Lichenous fungi | +++ | +++ |
| Scleroglucan | Linear β-1,3-glucan | Fungal | +++ | ++ |
| | with β-1,6- linked | exopolysaccharide | | |
| | Glc decorations | | | |
| Yeast | Linear β-1,3-glucan | Fungal cell wall | ++ | ++ |
| β-glucan | with β-1,6- linked | extract | | |
| | Glc decorations | | | |
| Curdlan | Linear β-1,3-glucan | Bacteria | − | − |
| Lichenan | Linear mixed- | Moss | − | − |
| | linkage β-1,3- and | | | |
| | β-1,4-glucan | | | |
| Starch | Complex α-glucan | Plants | − | − |
| Birchwood | Linear β-1,4- xylan | Plants | − | − |
| xylan | with α-1,2-linked | | | |
| | GlcA decorations | | | |
| Avicel | Crystalline | Plants (chemically | − | − |
| cellulose | cellulose (β-1,4- | modified for partial | | |
| | glucan) | solubilisation) | | |
| Laminarin | Linear β-1,3-glucan | Brown algae | + | + |
| | with β-1,6- linked | | | |
| | Glc decorations | | | |

### Example 4. CMB92 Domain Proteins of the Present Disclosure can Bind Three Scleroglucan Molecules

Sequence analysis of Cp-F1 (SEQ ID NO:3, SEQ ID NO:20) and Cp-F2 (SEQ ID NO:4, SEQ ID NO:21) as well as its homologues) showed three repeat regions, SEQ ID NOs: 8, 10 and 12 for Cp-F1, and SEQ ID Nos: 9, 11 and 13 for Cp-F2, each containing one conserved Tryptophan binding site. The same repeat regions were confirmed in the other proteins of the CBM92 family (the proteins corresponding to SEQ ID NO:22 to SEQ ID NO:33 of Table 1), each having three repeat regions corresponding to the amino acid sequences of SEQ ID NO:34 to SEQ ID NO:69. Structural analysis of the protein by X-ray crystallography showed three apparent binding pockets, two of which have been able to be crystallised with a Glucose monosaccharide ligand. All three putative binding sites have been explored and confirmed by site-directed mutagenesis. Rheological analysis of mutant variants of the Cp-F1 protein showed that all three binding sites must be functional for stable hydrogel formation to occur. For each mutation, the Tryptophan (W/Trp) in the binding site “WExF” motif was converted by site-directed mutagenesis to an Alanine (A/Ala), the experiments were performed with a final in-gel scleroglucan concentration of 4 g/L and 50% protein loading (i.e. final protein concentration of 2 g/L).

### Results:

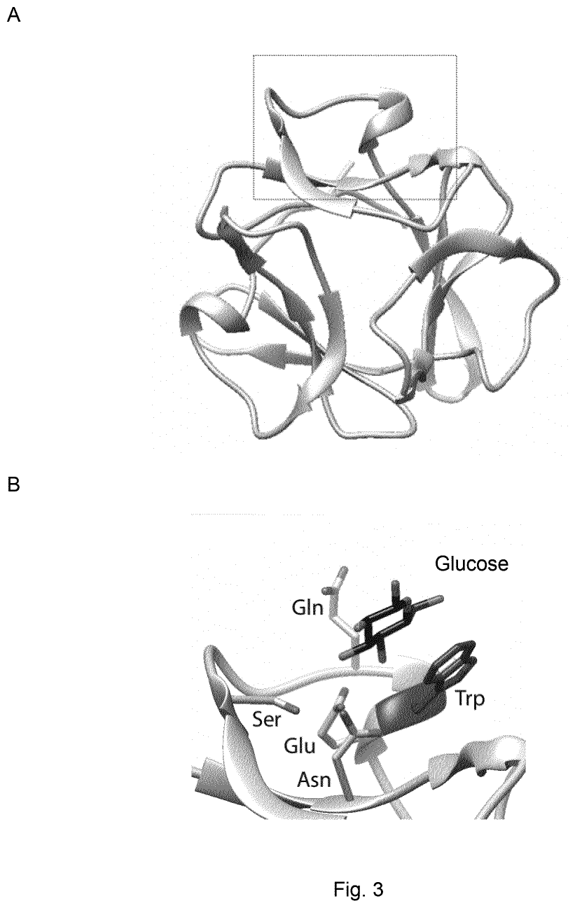

FIG. 2 shows the overlaid crystal structures of Cp-F1 and Cp-F2. Glucose monosaccharide ligands are visible in two of the binding sites. Binding and gel-formation was reduced in single and double mutants, and abolished in the triple mutant, confirming that three sites contribute to binding, and hence three polysaccharide chains can be bound. See also FIG. 3 (A and B) for an enlarged image of the binding site of repeat region 1 of Cp-F2 with some of the relevant amino acids indicated. Rheological data for mutant variants of Cp-F1 revealed that CP-F1 variants mutant No. 1 with Ala in the in the first binding site in the protein sequence (binding site
a, SEQ ID NO:5) and mutant No. 3 Ala in the third binding site in the protein sequence (binding site γ, SEQ ID NO:7) showed a very slight increase in polysaccharide viscosity, while mutant No. 2 Ala in the second binding site in the protein sequence (binding site β, SEQ ID NO:6) and a triple mutant (SEQ ID NO:19) were similar to a protein-free negative control.

### Example 5. CBM92 Domain Proteins of the Present Disclosure can Form a Hydrogel by Cross-Linking Scleroglucan

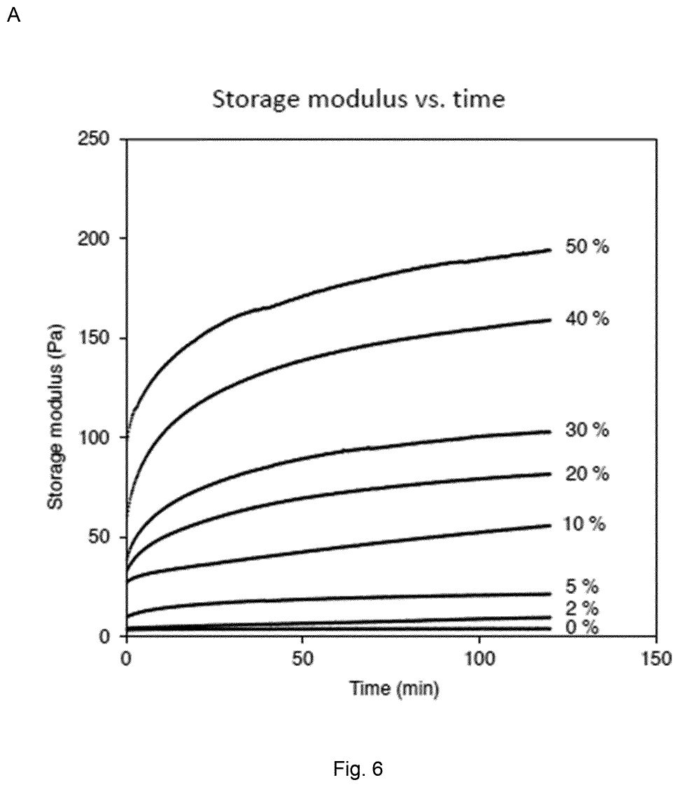

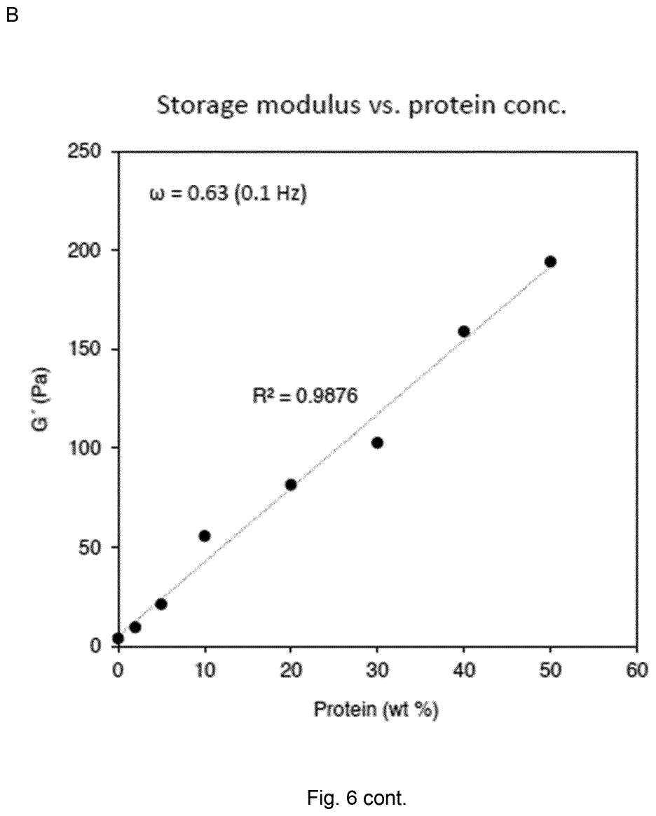

Protein Cp-F1 at a starting stock concentration of 4 mg/ml (higher starting stock concentrations can also be used) in water was mixed with scleroglucan at a starting stock concentration of 5 g/L in water at room temperature. The two solutions were mixed together quickly by adding the protein solution into a continuously vortexed polysaccharide solution. A hydrogel formed spontaneously within a few seconds and reached maximum stiffness after around one hour. In order to alter the stiffness of the hydrogel, we used a consistent stock concentration for both scleroglucan (e.g., 5 mg/ml as stock solution) and protein (4-30 mg/ml as stock solution), and mixed these with different ratios. The following final protein concentrations were tested: 0.08 g/L, 0.2 g/L, 0.4 g/L, 0.8 g/L, 1.2 g/L, 1.6 g/L and 2 g/L, which is equivalent to 2%, 5%, 10%, 20%, 30%, 40% and 50% of the protein content relative to the polysaccharide content respectively (see FIG. 6A). Scleroglucan was present at a final concentration of 4 g/L in all cases.

Another way would be to use a consistent scleroglucan concentration (e.g., 4 mg/ml in the final mixture, 5 mg/ml in the starting stock solution), and vary protein concentration by preparing differently concentrated protein stock solutions in advance.

Example: to produce 1 ml of hydrogel with final concentrations of 4 mg/ml scleroglucan and 2 mg/ml F1 protein, we mixed 0.8 ml scleroglucan (a solution at 5 mg/ml in water) with 0.2 ml F1 protein (a solution of 10 mg/ml in water).

Rheological properties of the hydrogel were measured at 25° C. using a rheometer with cone-plate configuration. Multiple concentrations and ratios of protein and polysaccharide were measured for each gel, always comparing to a rheology measurement for “polysaccharide without protein”. Both the shear viscosity and the dynamic shear properties, given by the storage modulus G′ and the loss modulus G″, were determined for each rheological experiment.

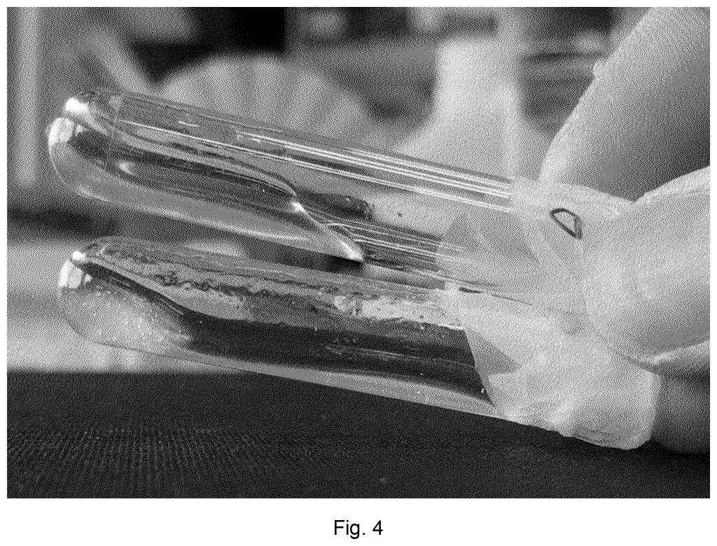

Results: When Cp-F1 or Cp-F2 were mixed with scleroglucan, a hydrogel formed spontaneously within a few minutes and almost reached maximum stiffness around one hour (FIG. 4). The storage modulus increased linearly with increasing protein amount, confirming that the protein function is responsible for gel stabilisation (FIG. 6A). The storage modulus of the hydrogel correlates linearly with increasing protein concentration (FIG. 6B). Analysis was repeated with other CMB92 domain proteins (Table 2), and all the wild type (native) proteins that showed binding to scleroglucan were also gel-forming with scleroglucan. In particular, all other CMB92 domain proteins listed in Table 2 and having a Tryptophan (W) in each of the three binding sites in the three repeat regions formed a reliable and stable hydrogel. Those proteins missing one or more Tryptophan (W) from one or more of the three binding sites in the three repeat regions may form unstable hydrogels. None of the Cp-F1 variants were gel-forming.

When increasing the protein:saccharide ratio, solidity of the gel increased and transparency decreased (FIG. 5); moreover, gel strength increased, as indicated in FIG. 6A.

### Sequence Overview

SEQ ID NO: 1 Full nucleic acid sequence encoding Cp-F1
SEQ ID NO: 2 Full nucleic acid sequence encoding Cp-F2
SEQ ID NO: 3 Full sequence for Cp-F1:
MASMTGGQQMGRGSVGKTIWLQGFNNKYVNSKNGQGAMWCDSDAPQAWELFTVV
DAGNGKIALRGNNGMYVSSENGEQAITCNRPAIQGWEAFDWLETADGKVSLRGSNG
LFISSENGAAAMTCTRPTASGWEAFGYSVVGNALEHHHHH
SEQ ID NO: 4 Full sequence for Cp-F2:
MPIGKTIWLQGFNSKYVNSRNGQGAMWCDSDTPQAWELFTVIDAGNGKIALRGNNG
LYVSSENGEQAMTCNRPAIDGWEVFDWISNSDGSVSLRGSNGMYVSSENGEQAITC
NRPAIDGWERFNWAAATALTGHHHHHHG
SEQ ID NO: 5 Full sequence for Cp-F1 with knocked-out
binding site in repeat region 1:
MASMTGGQQMGRGSVGKTIWLQGFNNKYVNSKNGQGAMWCDSDAPQAAELFTVV
DAGNGKIALRGNNGMYVSSENGEQAITCNRPAIQGWEAFDWLETADGKVSLRGSNG
LFISSENGAAAMTCTRPTASGWEAFGYSVVGNALEHHHHHH
SEQ ID NO: 6 Full sequence for Cp-F1 with knocked-out
binding site in repeat region 2:
MASMTGGQQMGRGSVGKTIWLQGFNNKYVNSKNGQGAMWCDSDAPQAWELFTVV
DAGNGKIALRGNNGMYVSSENGEQAITCNRPAIQGAEAFDWLETADGKVSLRGSNG
LFISSENGAAAMTCTRPTASGWEAFGYSVVGNALEHHHHHH
SEQ ID NO: 7 Full sequence for Cp-F1 with knocked-out
binding site in repeat region 3:
MASMTGGQQMGRGSVGKTIWLQGFNNKYVNSKNGQGAMWCDSDAPQAWELFTVV
DAGNGKIALRGNNGMYVSSENGEQAITCNRPAIQGWEAFDWLETADGKVSLRGSNG
LFISSENGAAAMTCTRPTASGAEAFGYSVVGNALEHHHHHH
SEQ ID NO: 8 Repeat region 1 of Cp-F1
IWLQGFNNKYVNSKNGQGAMWCDSDAPQAWELF
SEQ ID NO: 9 Repeat region 1 of Cp-F2
IWLQGFNSKYVNSRNGQGAMWCDSDTPQAWELF
SEQ ID NO: 10 Repeat region 2 of Cp-F1
IALRGNNGMYVSSENGEQAITCNRPAIQGWEAF
SEQ ID NO: 11 Repeat region 2 of Cp-F2
IALRGNNGLYVSSENGEQAMTCNRPAIDGWEVF
SEQ ID NO: 12 Repeat region 3 of Cp-F1
VSLRGSNGLFISSENGAAAMTCTRPTASGWEAF
SEQ ID NO: 13 Repeat region 3 of Cp-F2
VSLRGSNGMYVSSENGEQAITCNRPAIDGWERF
SEQ ID NO: 14 Repeat region 1-Consensus
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄X₂₅X₂₆X₂₇X₂₈X₂₉WEX₃₂F,
SEQ ID NO: 15 Repeat region 2-Consensus
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄X₂₅X₂₆X₂₇X₂₈X₂₉WEX₃₂F,
SEQ ID NO: 16 Repeat region 3-Consensus
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄X₂₅X₂₆X₂₇X₂₈X₂₉WEX₃₂F,
SEQ ID NO: 17 Double mutant, Cp-F1-W90AW132A, with 2^{nd} and
3^{rd} key Trp to Ala
MASMTGGQQMGRGSVGKTIWLQGFNNKYVNSKNGQGAMWCDSDAPQAWELFTVV
DAGNGKIALRGNNGMYVSSENGEQAITCNRPAIQGAEAFDWLETADGKVSLRGSNG
LFISSENGAAAMTCTRPTASGAEAFGYSVVGNALEHHHHHH
SEQ ID NO: 18 Double mutant, Cp-F1-W48AW132A, with 1^{st}
and 3^{rd} key Trp to Ala
MASMTGGQQMGRGSVGKTIWLQGFNNKYVNSKNGQGAMWCDSDAPQAAELFTVV
DAGNGKIALRGNNGMYVSSENGEQAITCNRPAIQGWEAFDWLETADGKVSLRGSNG
LFISSENGAAAMTCTRPTASGAEAFGYSVVGNALEHHHHHH
SEQ ID NO: 19 Triple mutant, Cp-F1-W48AW90AW132A
MASMTGGQQMGRGSVGKTIWLQGFNNKYVNSKNGQGAMWCDSDAPQAAELFTVV
DAGNGKIALRGNNGMYVSSENGEQAITCNRPAIQGAEAFDWLETADGKVSLRGSNG
LFISSENGAAAMTCTRPTASGAEAFGYSVVGNALEHHHHHH
SEQ ID NO: 20 Full sequence for Cp-F1
(without the N- and C-terminal tags)
VGKTIWLQGFNNKYVNSKNGQGAMWCDSDAPQAWELFTVVDAGNGKIALRGNNGM
YVSSENGEQAITCNRPAIQGWEAFDWLETADGKVSLRGSNGLFISSENGAAAMTCTR
PTASGWEAFGYSVVGNA
SEQ ID NO: 21 Full sequence for Cp-F2 without the C-terminal tags
PIGKTIWLQGFNSKYVNSRNGQGAMWCDSDTPQAWELFTVIDAGNGKIALRGNNGLY
VSSENGEQAMTCNRPAIDGWEVFDWISNSDGSVSLRGSNGMYVSSENGEQAITCNR
PAIDGWERFNWAAATALT
SEQ ID NO: 22 Full sequence for Aa-F1
IANRENISIRALSNNLYVSSENGLRPITCNRTKVDIWERFSIESVGGGRVAIKGSNGSYF
SSEDGLKPMRCNRKKAEAWEEFTLEPLGGDLYAIKGNNGAYVSHNNGSVEGLTCNK
SIVGDQEKFIIKDAASK
SEQ ID NO: 23 Full sequence for Aa-F2
TTDAPIGKTISLRKTNGDKKYVTAEQTANNKQLIARAIAVQGWEKFSIETHPDGGIALKA
LSNANYVQVQGINVNAAAAKKDKLTQFIWKNKGNGKVALKSVSANKWIQASWSSDNA
VLFAKGIEDKGWETFDWKIENTQK
SEQ ID NO: 24 Full sequence for Cp-F3
TGPIGQSVTLKGFNNQYVSSENGTQAMNCNRPTASGWETFSVVDAGGGKIALLSQG
KYVSSENGTQAMTCNRLTIGDWEKFDWVVNADGKISLRGSNGQYVSSENGTQPMN
CNRATISGWEAFGVNQ
SEQ ID NO: 25 Full sequence for Cp-F4
PVGQIITLRGSNNLYVSGENGTKAMTCERTAPQTWEQFSVWNAGPGKVNLRSMGKY
VSSENGLQAITCNRTTAASYEAFDWISNADGTVSLRGNNGLYVSSENGAAAMTCTRP
TIDGWEKFNFTIVGPA
SEQ ID NO: 26 Full sequence for Cp-F5
GGTAPIGSTITIKGSNGLFASGENGAQAMTCNRPTAQAWEQFTVVDAGGGKVALRSQ
GMYVSSENGAQAMTCSRPTIQDWEKFNWIDNGDGTFSLRGNNGSYVSSENGTQAM
TCNRPTIQGWEKFTR
SEQ ID NO: 27 Full sequence for Cp-F6
IGQTVTIKGFNNQYVCSEGNTQPMICNRAVAQSWEQFTVVDAGGGRVALRNQGNYV
CSENGTQAVNCNRASVGPWEQFEWISNSDGTISFRGNNGAYLSAEDGMARMTCTKT
TIGAAEKFKINQ
SEQ ID NO: 28 Full sequence for Cp-F7
VGSIIYLYHDTLLVCSENGTQAMNCNRTGLGPWEKFEVVDAGNNTVALKGNNGLYVK
AGNPVFCTGTALDSSTCFNWISLSSNTVALQSGKGLYMSSENGTQAMNWNRTAIGG
WETFRWGTTTAA
SEQ ID NO: 29 Full sequence for Ea-F1
PSIDIDEGKIALKSVHGKYLSAQPDGRAEWNRNIASEWEYFHLEKRQGDKITLKGAHG
MYVSAQPDGEVQINRQAAPPTGWEEFTVEDRGNNVICLKSIHWKYLSAQMDGTVQW
NRDSAPKGGWEEFQIVRPGGPG
SEQ ID NO: 30 Full sequence for Mr-F1
SGAPIGSTITLKGFNNQYVSSENGTQAMNCNRPTASAWEQFLVVDAGAGKIALQSMG
KYVSSENGTQAITCNRTTYGDWEKFDWVPTTDGKVTLRGNNGKFISSENGTQAMTC
TRATASGWEAFGVNQ
SEQ ID NO: 31 Full sequence for Mr-F2
PAPPIGTVISLKGFNGKYVSGENGTQAMTCNRTVAGDWEHFTVLDAGNGKIYLRSMG
KYVSSENGTQAITCNRTTPSDWEKFDWIVTTDGKITLRGNNGKFISSENGTQAMTCN
RTTASGWEAFGLNQ
SEQ ID NO: 32 Full sequence for Mr-F3
TPPIGQTITLKGSNNNYVSSENGTQPMNCNRPTAGGWEQFTIVNAGSGKVALLNSGK
YVSSENGTQAINCNRTSVGPWEQFDWVGNADGTVSFRGSNGKYISGENGTQAMTC
NRATIGGWESFRVNQ
SEQ ID NO: 33 Full sequence for Pc-F1
AVAAPVGQTIWLKACSTQQFVSADQNLGATAPLVANRATVQGWEQFQVVDAGGGTI
ALRATGSGLYVSADTNVGGQLTANRPTIQDWERFEWVELGNGSIGLKARSNGLYVSA
DLGRNASAPLYASRASIGGCWEAFTWGSVGG
SEQ ID NO: 34 Repeat region 1 of Aa-F1
ISIRALSNNLYVSSENGLRPITCNRTKVDIWERF
SEQ ID NO: 35 Repeat region 2 of Aa-F1
VAIKGSNGSYFSSEDGLKPMRCNRKKAEAWEEF
SEQ ID NO: 36 Repeat region 3 of Aa-F1
YAIKGNNGAYVSHNNGSVEGLTCNKSIVGDQEKF
SEQ ID NO: 37 Repeat region 1 of Aa-F2
ISLRKTNGDKKYVTAEQTANNKQLIARAIAVQGWEKF
SEQ ID NO: 38 Repeat region 2 of Aa-F2
IALKALSNANYVQVQGINVNAAAAKKDKLTQF
SEQ ID NO: 39 Repeat region 3 of Aa-F2
VALKSVSANKWIQASWSSDNAVLFAKGIEDKGWETF
SEQ ID NO: 40 Repeat region 1 of Cp-F3
VTLKGFNNQYVSSENGTQAMNCNRPTASGWETF
SEQ ID NO: 41 Repeat region 2 of Cp-F3
IALLSQGKYVSSENGTQAMTCNRLTIGDWEKF
SEQ ID NO: 42 Repeat region 3 of Cp-F3
ISLRGSNGQYVSSENGTQPMNCNRATISGWEAF
SEQ ID NO: 43 Repeat region 1 of Cp-F4
ITLRGSNNLYVSGENGTKAMTCERTAPQTWEQF
SEQ ID NO: 44 Repeat region 2 of Cp-F4
VNLRSMGKYVSSENGLQAITCNRTTAASYEAF
SEQ ID NO: 45 Repeat region 3 of Cp-F4
VSLRGNNGLYVSSENGAAAMTCTRPTIDGWEKF
SEQ ID NO: 46 Repeat region 1 of Cp-F5
ITIKGSNGLFASGENGAQAMTCNRPTAQAWEQF
SEQ ID NO: 47 Repeat region 2 of Cp-F5
VALRSQGMYVSSENGAQAMTCSRPTIQDWEKF
SEQ ID NO: 48 Repeat region 3 of Cp-F5
FSLRGNNGSYVSSENGTQAMTCNRPTIQGWEKF
SEQ ID NO: 49 Repeat region 1 of Cp-F6
VTIKGFNNQYVCSEGNTQPMICNRAVAQSWEQF
SEQ ID NO: 50 Repeat region 2 of Cp-F6
VALRNQGNYVCSENGTQAVNCNRASVGPWEQF
SEQ ID NO: 51 Repeat region 3 of Cp-F6
ISFRGNNGAYLSAEDGMARMTCTKTTIGAAEKF
SEQ ID NO: 52 Repeat region 1 of Cp-F7
IYLYHDTLLVCSENGTQAMNCNRTGLGPWEKF
SEQ ID NO: 53 Repeat region 2 of Cp-F7
VALKGNNGLYVKAGNPVFCTGTALDSSTCE
SEQ ID NO: 54 Repeat region 3 of Cp-F7
VALQSGKGLYMSSENGTQAMNWNRTAIGGWETF
SEQ ID NO: 55 Repeat region 1 of Ea-F1
IALKSVHGKYLSAQPDGRAEWNRNIASEWEYF
SEQ ID NO: 56 Repeat region 2 of Ea-F1
ITLKGAHGMYVSAQPDGEVQINRQAAPPTGWEEF
SEQ ID NO: 57 Repeat region 3 of Ea-F1
ICLKSIHWKYLSAQMDGTVQWNRDSAPKGGWEEF
SEQ ID NO: 58 Repeat region 1 of Mr-F1
ITLKGFNNQYVSSENGTQAMNCNRPTASAWEQF
SEQ ID NO: 59 Repeat region 2 of Mr-F1
IALQSMGKYVSSENGTQAITCNRTTYGDWEKF
SEQ ID NO: 60 Repeat region 3 of Mr-F1
VTLRGNNGKFISSENGTQAMTCTRATASGWEAF
SEQ ID NO: 61 Repeat region 1 of Mr-F2
ISLKGFNGKYVSGENGTQAMTCNRTVAGDWEHF
SEQ ID NO: 62 Repeat region 2 of Mr-F2
YLRSMGKYVSSENGTQAITCNRTTPSDWEKF
SEQ ID NO: 63 Repeat region 3 of Mr-F2
ITLRGNNGKFISSENGTQAMTCNRTTASGWEAF
SEQ ID NO: 64 Repeat region 1 of Mr-F3
ITLKGSNNNYVSSENGTQPMNCNRPTAGGWEQF
SEQ ID NO: 65 Repeat region 2 of Mr-F3
VALLNSGKYVSSENGTQAINCNRTSVGPWEQF
SEQ ID NO: 66 Repeat region 3 of Mr-F3
VSFRGSNGKYISGENGTQAMTCNRATIGGWESF
SEQ ID NO: 67 Repeat region 1 of Pc-F1
IWLKACSTQQFVSADQNLGATAPLVANRATVQGWEQF
SEQ ID NO: 68 Repeat region 2 of Pc-F1
IALRATGSGLYVSADTNVGGQLTANRPTIQDWERF
SEQ ID NO: 69 Repeat region 3 of Pc-F1
IGLKARSNGLYVSMEGGSSPINANRASIGGCWEAF
SEQ ID NO: 70 Full sequence for Dm-F1 non-binding,
non-gel forming protein
SYIGIGDNVWFEAYNGSFISSENGASPMTCNTFTVGETEVFTIVDAGDGKIALLGNNG
KYVSSNNGTTSMTCTKDEIGETEKFYWINLSNGQMALLGKGGFVSMEGGSSPINANR
NAIDGWEIYSWGKQDIQT
SEQ ID NO: 220 Repeat region 1-Consensus
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄X₂₅X₂₆X₂₇X₂₈X₂₉X₃₀X₃₁X₃₂X₃₃X₃₄WEX₃₇F,
SEQ ID NO: 221 Repeat region 2-Consensus
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄X₂₅X₂₆X₂₇X₂₈X₂₉X₃₀X₃₁WEX₃₄F,
SEQ ID NO: 222 Repeat region 3-Consensus
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄X₂₅X₂₆X₂₇X₂₈X₂₉X₃₀X₃₁X₃₂X₃₃X₃₄X₃₅WEX₃₈F,

### REFERENCES

Boraston A B, Bolam D N, Gilbert H J, Davies G J. Carbohydrate-binding modules: fine-tuning polysaccharide recognition. Biochem J. 2004; 382(Pt 3):769-81.
Coviello T, Grassi M, Rambone G, Santucci E, Carafa M, Murtas E, et al. Novel hydrogel system from scleroglucan:synthesis and characterization. Journal of Controlled Release. 1999; 60(2):367-78
Coviello T, Grassi M, Rambone G, Alhaique F. A crosslinked system from Scleroglucan derivative: preparation and characterization. Biomaterials. 2001; 22(13):1899-909
Coviello T, Grassi M, Palleschi A, Bocchinfuso G, Coluzzi G, Banishoeib F, et al. A new scleroglucan/borax hydrogel: swelling and drug release studies. International Journal of Pharmaceutics. 2005; 289(1):97-107
Crescenzi V, Gamini A, Paradossi G, Torri G. Solution properties of a new polyelectrolyte derived from the polysaccharide scleroglucan. Carbohydr Pol. 1983; 3(4):273-86
Lombard V, Golaconda Ramulu H, Drula E, Coutinho P M, Henrissat B. The carbohydrate-active enzymes database (CAZy) in 2013. Nucleic Acids Res. 2014; 42(Database issue):D490-5
Maeda H, Rambone G, Coviello T, Yuguchi Y, Urakawa H, Alhaique F, et al. Low-degree oxidized scleroglucan and its hydrogel. International Journal of Biological Macromolecules. 2001; 28(5):351-8
Palleschi A, Bocchinfuso G, Coviello T, Alhaique F. Molecular dynamics investigations of the polysaccharide scleroglucan: first study on the triple helix structure. Carbohydr Res. 2005; 340(13):2154-62
Palleschi A, Coviello T, Bocchinfuso G, Alhaique F. Investigation on a new scleroglucan/borax hydrogel: Structure and drug release. International Journal of Pharmaceutics. 2006; 322(1):13-21
Schmid J, Meyer V, Sieber V. Scleroglucan: biosynthesis, production and application of a versatile hydrocolloid. Appl Microbiol Biotechnol. 2011; 91(4):937-47
Tomme P., Boraston A., Kormos J. M. et al. Affinity electrophoresis for the identification and characterization of soluble sugar binding by carbohydrate-binding modules. Enzyme and Microbial Technology 2000:27(7):453-458.

### Items

1. A polypeptide comprising at least two repeat regions selected from the group consisting of:
   a. a repeat region 1 (R1), comprising or consisting of an amino acid sequence

SEQ ID NO: X:
I W L Q G F N N K Y V N S K N G Q G A M W
1 2 3 4 5 6 7 8 9 10 11 12 13 14 15 16 17 18 19 20 21
C D S D A P Q A W E L F
22 23 24 25 26 27 28 29 30 31 32 33

or a functional variant thereof wherein the amino acid sequence of said variant differs from SEQ ID NO:X in that the amino acid sequence of the variant comprises 1 to 6 individual amino acid substitutions,
b. a repeat region 2 (R2), comprising or consisting of an amino acid sequence

SEQ ID NO: XX:
I A L R G N N G M Y V S S E N G E Q A I T
1 2 3 4 5 6 7 8 9 10 11 12 13 14 15 16 17 18 19 20 21
C N R P A I Q G W E A F
22 23 24 25 26 27 28 29 30 31 32 33

or a functional variant thereof wherein the amino acid sequence of said variant differs from SEQ ID NO: 10 in that the amino acid sequence of the variant comprises 1 to 6 individual amino acid substitutions,
c. a repeat region 3 (R3), comprising or consisting of an amino acid sequence

SEQ ID NO: XXX:
V S L R G S N G L F I S S E N G A A A M
1 2 3 4 5 6 7 8 9 10 11 12 13 14 15 16 17 18 19 20
T C T R P T A S G W E A F
21 22 23 24 25 26 27 28 29 30 31 32 33

or a functional variant thereof wherein the amino acid sequence of said variant differs from SEQ ID NO: XXX in that the amino acid sequence of the variant comprises 1 to 6 individual amino acid substitutions.
2. A polypeptide comprising at least two repeat regions selected from the group consisting of:
   a. a repeat region 1 (R1), comprising or consisting of an amino acid sequence

SEQ ID NO: 14
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉WEX₃₂F,

wherein
X₁ is Isoleucine (I) or Valine (V);
X₂ is any amino acid, such as Tryptophan (W), Serine (S), Threonine (T) or Alanine (A);
X₃ is Leucine (L), Isoleucine (I) or Phenylalanine (F);
X₄ is Glutamine (Q), Arginine (R) or Lysine (K);
X₅ is Glycine (G), Alanine (A), Serine (S), Asparagine (N) or Lysine (K);
X₆ is any amino acid, such as Alanine (A);
X₇ is Asparagine (N), Histidine (H), Serine (S) or Threonine (T);
X₈ is Asparagine (N) or Glycine (G);
X₉ is Lysine (K), Glutamine (Q) or Leucine (L);
X₁₀ is Tyrosine (Y) or Phenylalanine (F);
X₁₁ is Valine (V), Leucine (L), Isoleucine (I) or Methionine (M);
X₁₂ is Serine (S), Threonine (T), Cysteine (C) or Asparagine (N);
X₁₃ is Alanine (A), Serine (S) or Glycine (G);
X₁₄ is Glutamic acid (E) or Aspartic acid (D);
X₁₅ is Asparagine (N) or Aspartic acid (D);
X₁₆ is Glycine (G), Proline (P), Asparagine (N) or Valine (V);
X₁₇ is any amino acid, such as Threonine (T);
X₁₈ is any amino acid, such as Glycine (G);
X₁₉ is Proline (P), Alanine (A) or Arginine (R);
X₂₀ is Leucine (L), Methionine (M), Alanine (A), Valine (V) or Isoleucine (I);
X₂₁ is any amino acid, such as Threonine (T);
X₂₂ is Alanine (A), Cysteine (C) or Tryptophan (W);
X₂₃ is Asparagine (N), Aspartic acid (D), Threonine (T) or Serine (S);
X₂₄ is Arginine (R), Alanine (A) or Serine (S);
X₂₅ is Threonine (T), Aspartic acid (D) or Alanine (A);
X₂₆ is Threonine (T), Alanine (A) or Serine (S);
X₂₇ is Alanine (A), Valine (V) or Isoleucine (I);
X₂₈ is Glycine (G), Glutamine (Q), Serine (S) or Aspartic acid (D);
X₂₉ is any amino acid, such as Glycine (G);
X₃₂ is any amino acid, such as Glutamine (Q) or Lysine (K);
or a functional variant thereof wherein the amino acid sequence of said variant differs from SEQ ID NO: 14 in that the amino acid sequence of the variant comprises 1 to 6 individual amino acid substitutions,
b. a repeat region 2 (R2), comprising or consisting of an amino acid sequence

SEQ ID NO: 15:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉WEX₃₂F,

wherein
X₁ is Isoleucine (I), Valine (V) or Phenylalanine (F);
X₂ is Alanine (A), Serine (S) or Threonine (T);
X₃ is Leucine (L) or Phenylalanine (F);
X₄ is Glutamine (Q), Arginine (R) or Lysine (K);
X₅ is Glycine (G), Alanine (A) or Serine (S);
X₆ is any amino acid, such as Alanine (A);
X₇ is Asparagine (N), Methionine (M), Histidine (H) or Threonine (T);
X₈ is Glycine (G), Asparagine (N) or Aspartic Acid (D);
X₉ is Lysine (K), Leucine (L), Methionine (M) or Glutamine (Q);
X₁₀ is Tyrosine (Y) or Phenylalanine (F);
X₁₁ is Valine (V), Leucine (L) or Isoleucine (I);
X₁₂ is Serine (S), Cysteine (C) or Threonine (T);
X₁₃ is Alanine (A) or Serine (S);
X₁₄ is Glutamic acid (E), Aspartic acid (D), Glutamine (Q) or Asparagine (N);
X₁₅ is Asparagine (N), Proline (P), Glycine (G), Threonine (T), Glutamine (Q) or Aspartic acid (D);
X₁₆ is Glycine (G), Aspartic Acid (D), Alanine (A), Asparagine (N), Threonine (T) or Arginine (R);
X₁₇ is Glycine (G), Threonine (T), Leucine (L), Alanine (A), Valine (V) or Glutamine (Q);
X₁₈ is any amino acid, such as Serine (S);
X₁₉ is Proline (P), Alanine (A), Glutamine (Q) or Lysine (K);
X₂₀ is Leucine (L), Isoleucine (I), Methionine (M) or Valine (V);
X₂₁ is Threonine (T), Isoleucine (I), Asparagine (N), Valine (V) or Alanine (A);
X₂₂ is Alanine (A), Cysteine (C) or Tryptophan (W);
X₂₃ is Asparagine (N), Aspartic acid (D), Threonine (T) or Serine (S);
X₂₄ is Alanine (A), Cysteine (C) or Arginine (R);
X₂₅ is Threonine (T), Alanine (A), Asparagine (N), Proline (P) or Aspartic acid (D);
X₂₆ is any amino acid, such as Alanine (A);
X₂₇ is Isoleucine (I), Valine (V), Proline (P), Alanine (A), or Leucine (L);
X₂₈ is Glycine (G), Serine (S), Glutamine (Q), or Aspartic acid (D);
X₂₉ is Glycine (G), Alanine (A), Aspartic Acid (D), Glutamic Acid (E), Proline (P) or Arginine (R);
X₃₂ is any amino acid, such as Lysine (K);
or a functional variant thereof wherein the amino acid sequence of said variant differs from SEQ ID NO: 15 in that the amino acid sequence of the variant comprises 1 to 6 individual amino acid substitutions,
c. a repeat region 3 (R3), comprising or consisting of an amino acid sequence

SEQ ID NO: 16:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉WEX₃₂F,

wherein
X₁ is Valine (V), Isoleucine (I), Tyrosine (Y), Phenylalanine (F) or Glutamic Acid (E);
X₂ is Alanine (A), Serine (S), Cysteine (C) or Glycine (G);
X₃ is Leucine (L), Phenylalanine (F) or Isoleucine (I);
X₄ is Arginine (R), Lysine (K), Glutamine (Q), Leucine (L) or Methionine (M),
X₅ is Glycine (G), Alanine (A) or Serine (S);
X₆ is any amino acid, such as Asparagine (N);
X₇ is Asparagine (N), Histidine (H) or Threonine (T);
X₈ is Glycine (G) or Asparagine (N);
X₉ is Lysine (K), Leucine (L), Asparagine (R), Glutamine (Q) or Asparagine (N);
X₁₀ is Tyrosine (Y) or Phenylalanine (F);
X₁₁ is Valine (V), Leucine (L) or Isoleucine (I);
X₁₂ is Serine (S), Threonine (T), Cysteine (C) or Glutamine (Q);
X₁₃ is Alanine (A), Serine (S), Histidine (H), Threonine (T), Methionine (M) or Glutamine (Q);
X₁₄ is Glutamic acid (E), Aspartic acid (D), Glutamine (Q) or Asparagine (N);
X₁₅ is Asparagine (N), Leucine (L) or Aspartic acid (D);
X₁₆ is Glycine (G), Asparagine (N), Alanine (A), or Aspartic Acid (D);
X₁₇ is any amino acid, such as Glycine (G);
X₁₈ is any amino acid, such as Alanine (A);
X₁₉ is Proline (P), Alanine (A), Glutamine (Q) or Glycine (G);
X₂₀ is Leucine (L), Methionine (M), Glutamine (Q), Valine (V) or Isoleucine (I);
X₂₁ is Threonine (T), Isoleucine (I), Asparagine (N), Leucine (L) or Methionine (M);
X₂₂ is Alanine (A), Cysteine (C) or Arginine (R);
X₂₃ is Asparagine (N), Aspartic acid (D), Threonine (T) or Serine (S);
X₂₄ is Arginine (R), Histidine (H), Alanine (A), Serine (S) or Lysine (K);
X₂₅ is Threonine (T), Alanine (A), Proline (P), Serine (S) or Aspartic acid (D);
X₂₆ is any amino acid, such as Alanine (A);
X₂₇ is Isoleucine (I), Alanine (A) or Valine (V);
X₂₈ is Glycine (G), Serine (S) or Glutamine (Q);
X₂₉ is Glycine (G), Alanine (A), Proline (P) or Cysteine (C);
X₃₂ is any amino acid, such as Lysine (K);
or a functional variant thereof wherein the amino acid sequence of said variant differs from SEQ ID NO: 16 in that the amino acid sequence of the variant comprises 1 to 6 individual amino acid substitutions.

1. A polypeptide comprising at least two repeat regions selected from the group consisting of:
a. a repeat region 1 (R1), comprising or consisting of an amino acid sequence
SEQ ID NO: 8:
I W L — Q G F — N N K Y V N S K N G — —
1 2 3 4 5 6 7 8 9 10 11 12 13 14 15 16 17 18 19 20
— Q G A M W C D S D A P Q A W E L
21 22 23 24 25 26 27 28 29 30 31 32 33 34 35 36 37
F
38
or a functional variant thereof wherein the amino acid sequence of said variant differs from SEQ ID NO:8 in that the amino acid sequence of the variant comprises 1 to 6 individual amino acid substitutions,
b. a repeat region 2 (R2), comprising or consisting of an amino acid sequence
SEQ ID NO: 10:
I A L R G N — N G M Y V S S E N G - E Q
1 2 3 4 5 6 7 8 9 10 11 12 13 14 15 16 17 18 19 20
A I T C N R P A I Q G W E A F
21 22 23 24 25 26 27 28 29 30 31 32 33 34 35
or a functional variant thereof wherein the amino acid sequence of said variant differs from SEQ ID NO: 10 in that the amino acid sequence of the variant comprises 1 to 6 individual amino acid substitutions,
c. a repeat region 3 (R3), comprising or consisting of an amino acid sequence
SEQ ID NO: 12:
V S L R G — S N G L F I S S E N G A — —
1 2 3 4 5 6 7 8 9 10 11 12 13 14 15 16 17 18 19 20
— A A M T C T R — P T A — S G W E
21 22 23 24 25 26 27 28 29 30 31 32 33 34 35 36 37
A F
38 39
or a functional variant thereof wherein the amino acid sequence of said variant differs from SEQ ID NO: 12 in that the amino acid sequence of the variant comprises 1 to 6 individual amino acid substitutions.

2. A polypeptide comprising at least two repeat regions selected from the group consisting of:
a. a repeat region 1 (R1), comprising or consisting of an amino acid sequence
SEQ ID NO: 220:
X₁X₂X₃X₄X₅X₆X₇X₈X₉X₁₀X₁₁X₁₂X₁₃X₁₄X₁₅X₁₆X₁₇X₁₈X₁₉X₂₀
X₂₁X₂₂X₂₃X₂₄X₂₅X₂₆X₂₇X₂₈X₂₉X₃₀X₃₁X₃₂X₃₃X₃₄WEX₃₇F,
wherein
X₁ is Isoleucine (I) or Valine (V);
X₂ is any amino acid, such as Tryptophan (W), Serine (S), Threonine (T) or Alanine (A);
X₃ is Leucine (L) or Isoleucine (I);
X₄ is Lysine (K), or Arginine (R), or is omitted;
X₅ is Glutamine (Q), Arginine (R), Alanine (A) or Lysine (K), or omitted;
X₆ is Glycine (G), Serine (S), Tyrosine (Y), Cysteine (C), Leucine (L) or Threonine (T);
X₇ is any amino acid, such as Phenylalanine (F) or Serine (S);
X₈ is Glycine (G), or is omitted;
X₉ is Asparagine (N), Histidine (H), Aspartic acid (D) or Threonine (T);
X₁₀ is Asparagine (N), Serine (S), Threonine (T), Glutamine (Q), Lysine (K) or Glycine (G);
X₁₁ is Lysine (K), Glutamine (Q), Asparagine (N) or Leucine (L);
X₁₂ is Tyrosine (Y), Leucine (L) or Phenylalanine (F);
X₁₃ is Valine (V), Leucine (L) or Alanine (A);
X₁₄ is Serine (S), Threonine (T), Cysteine (C) or Asparagine (N);
X₁₅ is Alanine (A), Serine (S) or Glycine (G);
X₁₆ is Glutamic acid (E), Lysine (K), Arginine (R), Glutamine (Q) or Aspartic acid (D);
X₁₇ is Asparagine (N), Glycine (G), Glutamine (Q) or Proline (P);
X₁₈ is Glycine (G), Asparagine (N), Threonine (T) or Aspartic acid (D);
X₁₉ is Leucine (L) or is omitted;
X₂₀ is Glycine (G) or Alanine (A), or is omitted;
X₂₁ is Alanine (A) or Asparagine (N), or is omitted;
X₂₂ is any amino acid, such as Threonine (T), or is omitted;
X₂₃ is any amino acid, such as Glycine (G);
X₂₄ is Proline (P), Alanine (A), Glutamine (Q) or Arginine (R);
X₂₅ is Leucine (L), Methionine (M), Alanine (A) or Isoleucine (I);
X₂₆ is any amino acid, such as Threonine (T);
X₂₇ is Alanine (A), Cysteine (C) or Tryptophan (W);
X₂₈ is Asparagine (N), Aspartic acid (D), Glutamic acid (E) or Arginine (R);
X₂₉ is Arginine (R), Alanine (A) or Serine (S);
X₃₀ is Threonine (T), Aspartic acid (D), Proline (P), Isoleucine (I), Asparagine (N) or Alanine (A);
X₃₁ is Threonine (T), Alanine (A), Valine (V), Glycine (G), Lysine (K) or Isoleucine (I);
X₃₂ is Alanine (A), Valine (V), Proline (P) or Leucine (L);
X₃₃ is Glycine (G), Glutamine (Q), Serine (S) or Aspartic acid (D);
X₃₄ is any amino acid, such as Glycine (G);
X₃₇ is any amino acid, such as Glutamine (Q) or Lysine (K);
or a functional variant thereof wherein the amino acid sequence of said variant differs from SEQ ID NO:220 in that the amino acid sequence of the variant comprises 1 to 6 individual amino acid substitutions,
b. a repeat region 2 (R2), comprising or consisting of an amino acid sequence
SEQ ID NO: 221:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄
X₂₅X₂₆X₂₇X₂₈X₂₉X₃₀X₃₁WEX₃₄F,
wherein
X₁ is Isoleucine (I) or Valine (V);
X₂ is Alanine (A), Asparagine (N), Tyrosine (Y) or Threonine (T);
X₃ is Leucine (L) or Isoleucine (I);
X₄ is Glutamine (Q), Arginine (R), Leucine (L) or Lysine (K);
X₅ is Glycine (G), Alanine (A), Asparagine (N) or Serine (S);
X₆ is any amino acid, such as Alanine (A), or is omitted;
X₇ is Glycine (G) or Serine (S), or is omitted;
X₈ is Asparagine (N), Methionine (M), Glutamine (Q), Histidine (H) or Serine (S);
X₉ is Glycine (G) or Alanine (A);
X₁₀ is Lysine (K), Leucine (L), Methionine (M), Asparagine (N) or Serine (S);
X₁₁ is Tyrosine (Y);
X₁₂ is Valine (V) or Phenylalanine (F);
X₁₃ is Serine (S), Cysteine (C), Lysine (K) or Glutamine (Q);
X₁₄ is Alanine (A), Valine (V) or Serine (S);
X₁₅ is Glutamic acid (E), Aspartic acid (D), Glutamine (Q) or Glycine (G);
X₁₆ is Asparagine (N), Proline (P), Glycine (G), Threonine (T) or Aspartic acid (D);
X₁₇ is Glycine (G), Aspartic Acid (D), Isoleucine (I) or Asparagine (N), or is omitted;
X₁₈ is Valine (V) or Glycine (G), or is omitted;
X₁₉ is Glycine (G), Threonine (T), Leucine (L), Alanine (A), or Glutamic acid (E) or is omitted;
X₂₀ is any amino acid, such as Glutamine (Q), or omitted;
X₂₁ is Proline (P), Alanine (A), Glutamine (Q) or Asparagine (N);
X₂₂ is Leucine (L), Isoleucine (I), Methionine (M) or Valine (V);
X₂₃ is Threonine (T), Asparagine (N), Phenylalanine (F) or Arginine (R);
X₂₄ is Alanine (A), Cysteine (C) or Arginine (R);
X₂₅ is Asparagine (N), Alanine (A), Threonine (T) or Glutamine (Q);
X₂₆ is Alanine (A), Glycine (G) or Arginine (R);
X₂₇ is Threonine (T), Alanine (A), Lysine (K), Proline (P) or Leucine (L);
X₂₈ is any amino acid, such as Alanine (A);
X₂₉ is Isoleucine (I), Valine (V), Proline (P), Alanine (A), Lysine (K), Tyrosine (Y) or Leucine (L);
X₃₀ is Glycine (G), Alanine (A), Glutamic acid (E), Threonine (T), Serine (S), Glutamine (Q) or Aspartic acid (D);
X₃₁ is Glycine (G), Alanine (A), Aspartic Acid (D), Serine (S), Proline (P) or Lysine (K);
X₃₄ is any amino acid, such as Lysine (K);
or a functional variant thereof wherein the amino acid sequence of said variant differs from SEQ ID NO:221 in that the amino acid sequence of the variant comprises 1 to 6 individual amino acid substitutions,
c. a repeat region 3 (R3), comprising or consisting of an amino acid sequence
SEQ ID NO: 222:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄
X₂₅X₂₆X₂₇X₂₈X₂₉X₃₀X₃₁X₃₂X₃₃X₃₄X₃₅WEX₃₈F,
wherein
X₁ is Valine (V), Isoleucine (I), Tyrosine (Y) or Phenylalanine (F);
X₂ is Alanine (A), Serine (S), Cysteine (C), Threonine (T) or Glycine (G);
X₃ is Leucine (L), Phenylalanine (F) or Isoleucine (I);
X₄ is Arginine (R), Lysine (K) or Glutamine (Q),
X₅ is Glycine (G), Alanine (A) or Serine (S);
X₆ is Arginine (R) or Valine (V), or is omitted;
X₇ is any amino acid, such as Asparagine (N);
X₈ is Asparagine (N), Histidine (H), Alanine (A), Lysine (K), or omitted;
X₉ is Glycine (G), Tryptophan (W) or Asparagine (N);
X₁₀ is Lysine (K), Leucine (L), Methionine (M), Serine (S), Alanine (A) or Glutamine (Q);
X₁₁ is Tyrosine (Y), Tryptophan (W) or Phenylalanine (F);
X₁₂ is Valine (V), Leucine (L), Methionine (M) or Isoleucine (I);
X₁₃ is Serine (S) or Glutamine (Q);
X₁₄ is Alanine (A), Serine (S), Histidine (H) or Glycine (G);
X₁₅ is Glutamic acid (E), Aspartic acid (D), Glutamine (Q), Serine (S) or Asparagine (N);
X₁₆ is Asparagine (N), Leucine (L), Tryptophan (W), Methionine (M) or Aspartic acid (D);
X₁₇ is Glycine (G), Serine (S) or Aspartic Acid (D);
X₁₈ is any amino acid, such as Serine (S) or Threonine (T);
X₁₉ is Asparagine (N), Valine (V) or Aspartic acid (D), or is omitted;
X₂₀ is Alanine (A), or is omitted;
X₂₁ is Serine (S), or is omitted;
X₂₂ is any amino acid, such as Alanine (A), or is omitted;
X₂₃ is Proline (P), Alanine (A), Arginine (R), Threonine (T) or Glycine (G);
X₂₄ is Leucine (L), Methionine (M), Valine (V) or Isoleucine (I);
X₂₅ is Threonine (T), Tyrosine (Y), Asparagine (N), Leucine (L) or Glutamine (Q);
X₂₆ is Alanine (A), Cysteine (C), Phenylalanine (F) or Tryptophan (W);
X₂₇ is Asparagine (N), Alanine (A), Threonine (T) or Serine (S);
X₂₈ is Arginine (R) or Lysine (K);
X₂₉ is Aspartic acid (D), or is omitted;
X₃₀ is Threonine (T), Alanine (A), Proline (P), Serine (S) or Glycine (G);
X₃₁ is any amino acid, such as Alanine (A);
X₃₂ is Isoleucine (I), Glutamic acid (E), Proline (P), Alanine (A) or Valine (V);
X₃₃ is Glycine (G), Lysine (K) or Aspartic acid (D), or is omitted;
X₃₄ is Glycine (G), Serine (S), Lysine (K), Aspartic acid (D) or Glutamine (Q);
X₃₅ is Glycine (G), Alanine (A), Aspartic acid (D) or Cysteine (C);
X₃₈ is any amino acid, such as Lysine (K);
or a functional variant thereof wherein the amino acid sequence of said variant differs from SEQ ID NO:222 in that the amino acid sequence of the variant comprises 1 to 6 individual amino acid substitutions.

3. The polypeptide according to any one of the preceding claims, wherein said polypeptide comprises a repeat region 1, comprising an amino acid sequence SEQ ID NO:8, SEQ ID NO:9, SEQ ID NO:34, SEQ ID NO:37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO:64, SEQ ID NO:67 or SEQ ID NO: 220, or a functional variant thereof wherein the amino acid sequence of said variant differs from SEQ ID NO:8, SEQ ID NO:9, SEQ ID NO:34, SEQ ID NO: 37, SEQ ID NO:40, SEQ ID NO: 43, SEQ ID NO:46, SEQ ID NO:49, SEQ ID NO: 52, SEQ ID NO:55, SEQ ID NO:58, SEQ ID NO:61, SEQ ID NO:64, SEQ ID NO: 67 or SEQ ID NO:220 in that the amino acid sequence of the variant comprises 1 to 6 individual amino acid substitutions,
with the proviso that the residue at position 35 of said functional variant of SEQ ID NO: 8, SEQ ID NO:9, SEQ ID NO:34, SEQ ID NO:37, SEQ ID NO:40, SEQ ID NO: 43, SEQ ID NO:46, SEQ ID NO:49, SEQ ID NO:52, SEQ ID NO:55, SEQ ID NO: 58, SEQ ID NO:61, SEQ ID NO:64, SEQ ID NO:67, SEQ ID NO:220 is a Tryptophan (W).

4. The polypeptide according to any one of the preceding claims, wherein said polypeptide comprises a repeat region 2, comprising an amino acid sequence SEQ ID NO: 10, SEQ ID NO: 11, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 or SEQ ID NO: 221, or a functional variant thereof wherein the amino acid sequence of said variant differs from SEQ ID NO: 10, SEQ ID NO:11, SEQ ID NO:35, SEQ ID NO: 38, SEQ ID NO:41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65, SEQ ID NO: 68 or SEQ ID NO:221 in that the amino acid sequence of the variant comprises 1 to 6 individual amino acid substitutions,
with the proviso that the residue at position 32 of said functional variant of SEQ ID NO: 10, SEQ ID NO: 11, SEQ ID NO:35, SEQ ID NO:38, SEQ ID NO:41, SEQ ID NO: 44, SEQ ID NO:47, SEQ ID NO:50, SEQ ID NO:53, SEQ ID NO:56, SEQ ID NO: 59, SEQ ID NO:62, SEQ ID NO:65, SEQ ID NO:68, SEQ ID NO:221 is a Tryptophan (W).

5. The polypeptide according to any one of the preceding claims, wherein said polypeptide comprises a repeat region 3, comprising an amino acid sequence SEQ ID NO: 12, SEQ ID NO: 13, SEQ ID NO:36, SEQ ID NO:39, SEQ ID NO: 42, SEQ ID NO:45, SEQ ID NO:48, SEQ ID NO:51, SEQ ID NO:54, SEQ ID NO: 57, SEQ ID NO: 60, SEQ ID NO: 63, SEQ ID NO:66, SEQ ID NO:69 or SEQ ID NO: 222, or a functional variant thereof wherein the amino acid sequence of said variant differs from SEQ ID NO: 12, SEQ ID NO: 13, SEQ ID NO:36, SEQ ID NO: 39, SEQ ID NO:42, SEQ ID NO:45, SEQ ID NO:48, SEQ ID NO:51, SEQ ID NO: 54, SEQ ID NO:57, SEQ ID NO:60, SEQ ID NO:63, SEQ ID NO:66, SEQ ID NO: 69 or SEQ ID NO:222 in that the amino acid sequence of the variant comprises 1 to 6 individual amino acid substitutions,
with the proviso that the residue at position 36 of said functional variant of SEQ ID NO: 12, SEQ ID NO: 13, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66 or SEQ ID NO:69, SEQ ID NO: 222 is a Tryptophan (W).

6. The polypeptide according to any one of the preceding claims, wherein said polypeptide comprises a repeat region 1, comprising an amino acid sequence SEQ ID NO:8, SEQ ID NO:9, SEQ ID NO:34, SEQ ID NO:37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO:64, SEQ ID NO:67 or SEQ ID NO: 220, or a functional variant thereof wherein the amino acid sequence of said variant differs from SEQ ID NO:8, SEQ ID NO:9, SEQ ID NO:34, SEQ ID NO: 37, SEQ ID NO: 40, SEQ ID NO: 43, SEQ ID NO:46, SEQ ID NO:49, SEQ ID NO: 52, SEQ ID NO:55, SEQ ID NO:58, SEQ ID NO:61, SEQ ID NO:64, SEQ ID NO: 67 or SEQ ID NO:220 in that the amino acid sequence of the variant comprises 1 to 6 individual amino acid substitutions,
with the proviso that the residue at position 35 of the functional variant of SEQ ID NO: 8, SEQ ID NO:9, SEQ ID NO:34, SEQ ID NO:37, SEQ ID NO:40, SEQ ID NO: 43, SEQ ID NO:46, SEQ ID NO:49, SEQ ID NO:52, SEQ ID NO:55, SEQ ID NO: 58, SEQ ID NO:61, SEQ ID NO:64, SEQ ID NO:67, SEQ ID NO:220 is a Tryptophan (W),
the residue at position 36 of the functional variant of SEQ ID NO:8, SEQ ID NO: 9, SEQ ID NO:220, SEQ ID NO:34, SEQ ID NO:37, SEQ ID NO:40, SEQ ID NO: 43, SEQ ID NO: 46, SEQ ID NO: 49, SEQ ID NO:52, SEQ ID NO:55, SEQ ID NO: 58, SEQ ID NO:61, SEQ ID NO:64, SEQ ID NO:67, is a Glutamic acid (E), and/or
the residue at position 38 of the functional variant of SEQ ID NO:8, SEQ ID NO: 9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67, SEQ ID NO:220 is a Phenylalanine (F).

7. The polypeptide according to any one of the preceding claims, wherein said polypeptide comprises a repeat region 2, comprising an amino acid sequence SEQ ID NO: 10, SEQ ID NO: 11, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 or SEQ ID NO: 221, or a functional variant thereof wherein the amino acid sequence of said variant differs from SEQ ID NO:10, SEQ ID NO: 11, SEQ ID NO:35, SEQ ID NO: 38, SEQ ID NO:41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65, SEQ ID NO: 68 or SEQ ID NO:221 in that the amino acid sequence of the variant comprises 1 to 6 individual amino acid substitutions,
with the proviso that
the residue at position 32 of the functional variant of SEQ ID NO: 10, SEQ ID NO: 11, SEQ ID NO:35, SEQ ID NO:38, SEQ ID NO:41, SEQ ID NO:44, SEQ ID NO: 47, SEQ ID NO: 50, SEQ ID NO:53, SEQ ID NO:56, SEQ ID NO:59, SEQ ID NO: 62, SEQ ID NO:65, SEQ ID NO:68, SEQ ID NO:221 is a Tryptophan (W), the residue at position 33 of the functional variant of SEQ ID NO: 10, SEQ ID NO: 11, SEQ ID NO: 35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO:68, SEQ ID NO:221 is a Glutamic acid (E), and/or
the residue at position 35 of the functional variant of SEQ ID NO: 10, SEQ ID NO: 11, SEQ ID NO:35, SEQ ID NO: 38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO:65, SEQ ID NO:68, SEQ ID NO:221 is a Phenylalanine (F).

8. The polypeptide according to any one of the preceding claims, wherein said polypeptide comprises a repeat region 3, comprising an amino acid sequence SEQ ID NO: 12, SEQ ID NO: 13, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69 or SEQ ID NO: 222, or a functional variant thereof wherein the amino acid sequence of said variant differs from SEQ ID NO: 12, SEQ ID NO: 13, SEQ ID NO:36, SEQ ID NO: 39, SEQ ID NO:42, SEQ ID NO:45, SEQ ID NO:48, SEQ ID NO:51, SEQ ID NO: 54, SEQ ID NO:57, SEQ ID NO:60, SEQ ID NO:63, SEQ ID NO:66, SEQ ID NO: 69 or SEQ ID NO:222 in that the amino acid sequence of the variant comprises 1 to 6 individual amino acid substitutions,
with the proviso that
the residue at position 36 of the functional variant of SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO:69, SEQ ID NO:222 is a Tryptophan (W), the residue at position 37 of the functional variant of SEQ ID NO: 12, SEQ ID NO: 13, SEQ ID NO: 36, SEQ ID NO: 39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO:69, SEQ ID NO:222 is a Glutamic acid (E), and/or
the residue at position 39 of the functional variant of SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO: 66, SEQ ID NO: 69, SEQ ID NO:222 is a Phenylalanine (F).

9. The polypeptide according to any one of the preceding claims, wherein said polypeptide comprises a repeat region 1, comprising an amino acid sequence SEQ ID NO:8, SEQ ID NO:9, SEQ ID NO:34, SEQ ID NO:37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO:64, SEQ ID NO:67 or SEQ ID NO: 220, or a functional variant thereof wherein the amino acid sequence of said variant differs from SEQ ID NO:8, SEQ ID NO:9, SEQ ID NO:34, SEQ ID NO: 37, SEQ ID NO:40, SEQ ID NO: 43, SEQ ID NO: 46, SEQ ID NO: 49, SEQ ID NO: 52, SEQ ID NO:55, SEQ ID NO:58, SEQ ID NO:61, SEQ ID NO:64, SEQ ID NO: 67 or SEQ ID NO:220 in that the amino acid sequence of the variant comprises 1 to 6 individual amino acid substitutions,
with the proviso that
the residue at position 12 of the functional variant of SEQ ID NO:8, SEQ ID NO: 9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67, SEQ ID NO:220 is a Tyrosine (Y), and/or
the residue at position 13 of the functional variant of SEQ ID NO:8, SEQ ID NO: 9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67, SEQ ID NO:220 is a Valine (V).

10. The polypeptide according to any one of the preceding claims, wherein said polypeptide comprises a repeat region 2, comprising an amino acid sequence SEQ ID NO:10, SEQ ID NO: 11, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 or SEQ ID NO: 221, or a functional variant thereof wherein the amino acid sequence of said variant differs from SEQ ID NO: 10, SEQ ID NO:11, SEQ ID NO:35, SEQ ID NO: 38, SEQ ID NO:41, SEQ ID NO: 44, SEQ ID NO:47, SEQ ID NO:50, SEQ ID NO: 53, SEQ ID NO:56, SEQ ID NO:59, SEQ ID NO:62, SEQ ID NO:65, SEQ ID NO: 68 or SEQ ID NO:221 in that the amino acid sequence of the variant comprises 1 to 6 individual amino acid substitutions,
with the proviso that
the residue at position 11 of the functional variant of SEQ ID NO: 10, SEQ ID NO: 11, SEQ ID NO:35, SEQ ID NO:38, SEQ ID NO:41, SEQ ID NO:44, SEQ ID NO: 47, SEQ ID NO: 50, SEQ ID NO: 53, SEQ ID NO:56, SEQ ID NO:59, SEQ ID NO: 62, SEQ ID NO:65, SEQ ID NO:68, SEQ ID NO:221 is a Tyrosine (Y), and/or
the residue at position 12 of the functional variant of SEQ ID NO: 10, SEQ ID NO: 11, SEQ ID NO:35, SEQ ID NO: 38, SEQ ID NO:41, SEQ ID NO:44, SEQ ID NO: 47, SEQ ID NO: 50, SEQ ID NO:53, SEQ ID NO:56, SEQ ID NO:59, SEQ ID NO: 62, SEQ ID NO:65, SEQ ID NO:68, SEQ ID NO:221 is a Valine (V).

11. The polypeptide according to any one of the preceding claims, wherein said polypeptide comprises a repeat region 3, comprising an amino acid sequence SEQ ID NO: 12, SEQ ID NO: 13, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, SEQ ID NO: 222, or a functional variant thereof wherein the amino acid sequence of said variant differs from SEQ ID NO: 12, SEQ ID NO: 13, SEQ ID NO:36, SEQ ID NO: 39, SEQ ID NO:42, SEQ ID NO:45, SEQ ID NO:48, SEQ ID NO:51, SEQ ID NO: 54, SEQ ID NO:57, SEQ ID NO: 60, SEQ ID NO:63, SEQ ID NO:66, SEQ ID NO: 69, SEQ ID NO: 222 in that the amino acid sequence of the variant comprises 1 to 6 individual amino acid substitutions,
with the proviso that
the residue at position 11 of the functional variant of SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO:69, SEQ ID NO:222 is a Tyrosine (Y), and/or
the residue at position 12 of the functional variant of SEQ ID NO: 12, SEQ ID NO: 13, SEQ ID NO:36, SEQ ID NO: 39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO: 66, SEQ ID NO:69, SEQ ID NO:222 is a Valine (V).

12. The polypeptide according to any one of the preceding claims, wherein repeat region 1 consists of an amino acid sequence SEQ ID NO:8, SEQ ID NO: 9, SEQ ID NO:34, SEQ ID NO:37, SEQ ID NO:40, SEQ ID NO:43, SEQ ID NO: 46, SEQ ID NO:49, SEQ ID NO:52, SEQ ID NO:55, SEQ ID NO:58, SEQ ID NO: 61, SEQ ID NO:64, SEQ ID NO:67, SEQ ID NO:220, or a functional variant thereof wherein the amino acid sequence of said variant differs from SEQ ID NO: 8, SEQ ID NO:9, SEQ ID NO:34, SEQ ID NO:37, SEQ ID NO:40, SEQ ID NO: 43, SEQ ID NO:46, SEQ ID NO:49, SEQ ID NO:52, SEQ ID NO:55, SEQ ID NO: 58, SEQ ID NO: 61, SEQ ID NO: 64, SEQ ID NO:67 or SEQ ID NO:220, in that the amino acid sequence of the variant comprises 1 to 6 individual amino acid substitutions,
repeat region 2 consists of an amino acid sequence SEQ ID NO:10, SEQ ID NO: 11, SEQ ID NO: 35, SEQ ID NO:38, SEQ ID NO:41, SEQ ID NO:44, SEQ ID NO: 47, SEQ ID NO:50, SEQ ID NO:53, SEQ ID NO:56, SEQ ID NO:59, SEQ ID NO: 62, SEQ ID NO: 65, SEQ ID NO:68, SEQ ID NO:221, or a functional variant thereof wherein the amino acid sequence of said variant differs from SEQ ID NO: 10, SEQ ID NO: 11, SEQ ID NO:35, SEQ ID NO:38, SEQ ID NO:41, SEQ ID NO: 44, SEQ ID NO:47, SEQ ID NO:50, SEQ ID NO:53, SEQ ID NO:56, SEQ ID NO: 59, SEQ ID NO:62, SEQ ID NO:65, SEQ ID NO:68 or SEQ ID NO:221 in that the amino acid sequence of the variant comprises 1 to 6 individual amino acid substitutions, and/or
repeat region 3 consists of an amino acid sequence SEQ ID NO: 12, SEQ ID NO: 13, SEQ ID NO:36, SEQ ID NO:39, SEQ ID NO:42, SEQ ID NO:45, SEQ ID NO: 48, SEQ ID NO:51, SEQ ID NO:54, SEQ ID NO:57, SEQ ID NO:60, SEQ ID NO: 63, SEQ ID NO:66, SEQ ID NO:69, SEQ ID NO:222 or a functional variant thereof wherein the amino acid sequence of said variant differs from SEQ ID NO: 12, SEQ ID NO: 13, SEQ ID NO:36, SEQ ID NO:39, SEQ ID NO:42, SEQ ID NO: 45, SEQ ID NO:48, SEQ ID NO:51, SEQ ID NO:54, SEQ ID NO:57, SEQ ID NO: 60, SEQ ID NO: 63, SEQ ID NO:66, SEQ ID NO:69 or SEQ ID NO:222, in that the amino acid sequence of the variant comprises 1 to 6 individual amino acid substitutions.

13. The polypeptide according to any one of the preceding claims, wherein said polypeptide comprises repeat region 1, repeat region 2 and repeat region 3.

14. The polypeptide according to any one of the preceding claims, wherein each of repeat region 1, repeat region 2 and repeat region 3 is flanked by at least three amino acid residues at its N-terminus and at least three amino acid residues at its C-terminus so that:
Y_{N}—R1-Y_{C}Z_{N}—R2-Z_{C}J_{N}-R3-J_{C},
wherein each of Y_{N}, Y_{C}, Z_{N}, Z_{C}, J_{N}, and J_{C} can individually be an amino acid sequence comprising at least three amino acids.

15. The polypeptide according to any one of the preceding claims, wherein the functional variant of SEQ ID NO:8, SEQ ID NO:9, SEQ ID NO:34, SEQ ID NO: 37, SEQ ID NO:40, SEQ ID NO:43, SEQ ID NO: 46, SEQ ID NO:49, SEQ ID NO: 52, SEQ ID NO: 55, SEQ ID NO: 58, SEQ ID NO:61, SEQ ID NO:64, SEQ ID NO: 67 or SEQ ID NO:220 differs from SEQ ID NO:8, SEQ ID NO:9, SEQ ID NO: 34, SEQ ID NO: 37, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220, in that the amino acid sequence of the variant comprises 5 individual amino acid substitutions, such as 4 individual amino acid substitutions, such as 3 individual amino acid substitutions, such as 2 individual amino acid substitutions, such as 1 individual amino acid substitution.

16. The polypeptide according to any one of the preceding claims, wherein the functional variant of SEQ ID NO:8, SEQ ID NO:9, SEQ ID NO:34, SEQ ID NO: 37, SEQ ID NO: 40, SEQ ID NO:43, SEQ ID NO:46, SEQ ID NO:49, SEQ ID NO: 52, SEQ ID NO:55, SEQ ID NO:58, SEQ ID NO:61, SEQ ID NO:64, SEQ ID NO: 67 or SEQ ID NO:220 differs from SEQ ID NO:8, SEQ ID NO:9, SEQ ID NO: 34, SEQ ID NO:37, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO: 64, SEQ ID NO:67 or SEQ ID NO:220 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 34 or 36 to 38 of SEQ ID NO:8, SEQ ID NO:9, SEQ ID NO:34, SEQ ID NO:37, SEQ ID NO: 40, SEQ ID NO:43, SEQ ID NO:46, SEQ ID NO:49, SEQ ID NO:52, SEQ ID NO:55, SEQ ID NO:58, SEQ ID NO:61, SEQ ID NO:64, SEQ ID NO:67 or SEQ ID NO:220, such as at any one of residues 1 to 34 or 37 of SEQ ID NO:8, SEQ ID NO:9, SEQ ID NO:34, SEQ ID NO:37, SEQ ID NO: 40, SEQ ID NO: 43, SEQ ID NO:46, SEQ ID NO:49, SEQ ID NO:52, SEQ ID NO: 55, SEQ ID NO:58, SEQ ID NO: 61, SEQ ID NO: 64, SEQ ID NO:67 or SEQ ID NO: 220.

17. The polypeptide according to any one of the preceding claims, wherein the functional variant of SEQ ID NO:10, SEQ ID NO:11, SEQ ID NO:35, SEQ ID NO: 38, SEQ ID NO:41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65, SEQ ID NO: 68 or SEQ ID NO:221, differs from SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO: 68 or SEQ ID NO:221 in that the amino acid sequence of the variant comprises 5 individual amino acid substitutions, such as 4 individual amino acid substitutions, such as 3 individual amino acid substitutions, such as 2 individual amino acid substitutions, such as 1 individual amino acid substitution.

18. The polypeptide according to any one of the preceding claims, wherein the functional variant of SEQ ID NO: 10, SEQ ID NO:11, SEQ ID NO:35, SEQ ID NO: 38, SEQ ID NO:41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65, SEQ ID NO: 68 or SEQ ID NO:221 differs from SEQ ID NO: 10, SEQ ID NO: 11, SEQ ID NO: 35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO: 65, SEQ ID NO:68 or SEQ ID NO:221 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 31 or 33 to 35 of SEQ ID NO: 10, SEQ ID NO:11, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 or SEQ ID NO:221, such as at any one of residues 1 to 31 or 34 of SEQ ID NO: 10, SEQ ID NO: 11, SEQ ID NO:35, SEQ ID NO:38, SEQ ID NO: 41, SEQ ID NO:44, SEQ ID NO:47, SEQ ID NO:50, SEQ ID NO:53, SEQ ID NO: 56, SEQ ID NO: 59, SEQ ID NO: 62, SEQ ID NO:65, SEQ ID NO: 68 or SEQ ID NO: 221.

19. The polypeptide according to any one of the preceding claims, wherein the functional variant of SEQ ID NO: 12, SEQ ID NO: 13, SEQ ID NO:36, SEQ ID NO: 39, SEQ ID NO:42, SEQ ID NO:45, SEQ ID NO:48, SEQ ID NO:51, SEQ ID NO: 54, SEQ ID NO:57, SEQ ID NO:60, SEQ ID NO:63, SEQ ID NO:66, SEQ ID NO: 69 or SEQ ID NO:222 differs from SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69 or SEQ ID NO:222 in that the amino acid sequence of the variant comprises 5 individual amino acid substitutions, such as 4 individual amino acid substitutions, such as 3 individual amino acid substitutions, such as 2 individual amino acid substitutions, such as 1 individual amino acid substitution.

20. The polypeptide according to any one of the preceding claims, wherein the functional variant of SEQ ID NO: 12, SEQ ID NO: 13, SEQ ID NO:36, SEQ ID NO: 39, SEQ ID NO:42, SEQ ID NO:45, SEQ ID NO:48, SEQ ID NO:51, SEQ ID NO: 54, SEQ ID NO:57, SEQ ID NO: 60, SEQ ID NO:63, SEQ ID NO:66, SEQ ID NO: 69 or SEQ ID NO:222 differs from SEQ ID NO: 12, SEQ ID NO: 13, SEQ ID NO: 36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO: 66, SEQ ID NO:69 or SEQ ID NO:222 in that the amino acid sequence of the variant comprises 6 individual amino acid substitutions, such as 5 individual amino acid substitutions, such as 4 individual amino acid substitutions, such as 3 individual amino acid substitutions, such as 2 individual amino acid substitutions, such as 1 individual amino acid substitution at any one of residues 1 to 35 or 37 to 39 of SEQ ID NO: 12, SEQ ID NO: 13, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69 or SEQ ID NO:222, such as at any one of residues 1 to 35 or 38 of SEQ ID NO: 12, SEQ ID NO: 13, SEQ ID NO:36, SEQ ID NO:39, SEQ ID NO: 42, SEQ ID NO:45, SEQ ID NO:48, SEQ ID NO:51, SEQ ID NO:54, SEQ ID NO: 57, SEQ ID NO: 60, SEQ ID NO: 63, SEQ ID NO:66, SEQ ID NO: 69 or SEQ ID NO: 222.

21. The polypeptide according to any one of the preceding claims, wherein repeat region 1 comprises or consists of an amino acid sequence of SEQ ID NO:8, SEQ ID NO:9, SEQ ID NO:34, SEQ ID NO:37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO:64, SEQ ID NO:67.

22. The polypeptide according to any one of the preceding claims, wherein repeat region 2 comprises or consists of an amino acid sequence of SEQ ID NO:10, SEQ ID NO: 11, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68.

23. The polypeptide according to any one of the preceding claims, wherein repeat region 3 comprises or consists of an amino acid sequence of SEQ ID NO: 12, SEQ ID NO: 13, SEQ ID NO:36, SEQ ID NO:39, SEQ ID NO:42, SEQ ID NO: 45, SEQ ID NO:48, SEQ ID NO:51, SEQ ID NO:54, SEQ ID NO:57, SEQ ID NO: 60, SEQ ID NO: 63, SEQ ID NO: 66, SEQ ID NO:69.

24. The polypeptide according to any one of the preceding claims, wherein said polypeptide comprises repeat region 1 and repeat region 2, or functional variants thereof; repeat region 1 and repeat region 3, or functional variants thereof; repeat region 2 and repeat region 3, or functional variants thereof; repeat region 1, repeat region 2 and repeat region 3, or functional variants thereof.

25. A polypeptide comprising an amino acid sequence having at least 75% sequence identity to SEQ ID NO:3, SEQ ID NO:4, SEQ ID NO:20 to SEQ ID NO: 33, or SEQ ID NO:71 to 219 and wherein said polypeptide is capable of binding one or more saccharide units.

26. A polypeptide comprising an amino acid sequence having at least 75% sequence identity to SEQ ID NO:3, SEQ ID NO:4, SEQ ID NO:20 to SEQ ID NO: 33, and wherein said polypeptide is capable of binding one or more saccharide units.

27. The polypeptide according to any one of claims 25 and 26, wherein said polypeptide comprises an amino acid sequence having at least 80% sequence identity to SEQ ID NO:3, SEQ ID NO:4, SEQ ID NO:20 to SEQ ID NO:33, or SEQ ID NO:71 to 218, such as at least 85% sequence identity to SEQ ID NO:3, SEQ ID NO:4, SEQ ID NO:20 to SEQ ID NO:33, or SEQ ID NO:71 to 219, such as at least 90% sequence identity to SEQ ID NO:3, SEQ ID NO:4, SEQ ID NO: 20 to SEQ ID NO:33, or SEQ ID NO:71 to 219, such as at least 95% sequence identity to SEQ ID NO:3, SEQ ID NO:4, SEQ ID NO:20 to SEQ ID NO: 33, or SEQ ID NO:71 to 219, such as at least 96% sequence identity to SEQ ID NO:3, SEQ ID NO:4, SEQ ID NO:20 to SEQ ID NO:33, or SEQ ID NO: 71 to 219, such as at least 97% sequence identity to SEQ ID NO:3, SEQ ID NO: 4, SEQ ID NO:20 to SEQ ID NO:33, or SEQ ID NO:71 to 219, such as at least 98% sequence identity to SEQ ID NO:3, SEQ ID NO:4, SEQ ID NO:20 to SEQ ID NO:33, or SEQ ID NO:71 to 219, such as at least 99% sequence identity to SEQ ID NO:3, SEQ ID NO:4, SEQ ID NO:20 to SEQ ID NO:33, or SEQ ID NO:71 to 219.

28. The polypeptide according to any one of claims 25 to 27, wherein said polypeptide comprises an amino acid sequence having at least 80% sequence identity to SEQ ID NO:3, SEQ ID NO:4 or SEQ ID NO:20 to SEQ ID NO:33, such as at least 85% sequence identity to SEQ ID NO:3, SEQ ID NO:4 or SEQ ID NO: 20 to SEQ ID NO:33, such as at least 90% sequence identity to SEQ ID NO: 3 SEQ ID NO:4 or SEQ ID NO:20 to SEQ ID NO:33, such as at least 95% sequence identity to SEQ ID NO:3, SEQ ID NO:4 or SEQ ID NO:20 to SEQ ID NO: 33, such as at least 96% sequence identity to SEQ ID NO:3, SEQ ID NO:4 or SEQ ID NO:20 to SEQ ID NO:33, such as at least 97% sequence identity to SEQ ID NO:3, SEQ ID NO:4 or SEQ ID NO:20 to SEQ ID NO:33, such as at least 98% sequence identity to SEQ ID NO:3, SEQ ID NO:4 or SEQ ID NO:20 to SEQ ID NO:33, such as at least 99% sequence identity to SEQ ID NO:3, SEQ ID NO:4 or SEQ ID NO:20 to SEQ ID NO:33.

29. The polypeptide according to any one of claims 25 to 28, wherein said polypeptide comprises or consists of amino acid sequence SEQ ID NO:3, SEQ ID NO: 4, SEQ ID NO:20 to SEQ ID NO:33, or SEQ ID NO:71 to 219.

30. The polypeptide according to any one of claims 25 to 29, wherein said polypeptide comprises at least two repeat regions selected from the group consisting of:
a. a repeat region 1, comprising or consisting of an amino acid sequence of SEQ ID NO:8, SEQ ID NO:9, SEQ ID NO:34, SEQ ID NO:37, SEQ ID NO: 40, SEQ ID NO: 43, SEQ ID NO:46, SEQ ID NO:49, SEQ ID NO:52, SEQ ID NO:55, SEQ ID NO:58, SEQ ID NO:61, SEQ ID NO:64 or SEQ ID NO: 67 or a functional variant thereof wherein the amino acid sequence of said variant differs from SEQ ID NO:8, SEQ ID NO:9, SEQ ID NO:34, SEQ ID NO: 37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO: 64 or SEQ ID NO: 67 in that the amino acid sequence of the variant comprises 1 to 6 individual amino acid substitutions;
b. a repeat region 2, comprising or consisting of an amino acid sequence of SEQ ID NO:10, SEQ ID NO:11, SEQ ID NO:35, SEQ ID NO:38, SEQ ID NO: 41, SEQ ID NO:44, SEQ ID NO:47, SEQ ID NO:50, SEQ ID NO:53, SEQ ID NO:56, SEQ ID NO:59, SEQ ID NO:62, SEQ ID NO:65 or SEQ ID NO: 68 or a functional variant thereof wherein the amino acid sequence of said variant differs from SEQ ID NO: 10, SEQ ID NO:11, SEQ ID NO:35, SEQ ID NO:38, SEQ ID NO:41, SEQ ID NO:44, SEQ ID NO:47, SEQ ID NO: 50, SEQ ID NO:53, SEQ ID NO:56, SEQ ID NO:59, SEQ ID NO:62, SEQ ID NO:65 or SEQ ID NO:68 in that the amino acid sequence of the variant comprises 1 to 6 individual amino acid substitutions; and
c. a repeat region 3, comprising or consisting of an amino acid sequence of SEQ ID NO: 12, SEQ ID NO: 13, SEQ ID NO:36, SEQ ID NO:39, SEQ ID NO: 42, SEQ ID NO: 45, SEQ ID NO:48, SEQ ID NO:51, SEQ ID NO:54, SEQ ID NO:57, SEQ ID NO:60, SEQ ID NO:63, SEQ ID NO:66 or SEQ ID NO: 69, or a functional variant thereof wherein the amino acid sequence of said variant differs from SEQ ID NO: 12, SEQ ID NO:13, SEQ ID NO:36, SEQ ID NO:39, SEQ ID NO:42, SEQ ID NO:45, SEQ ID NO:48, SEQ ID NO: 51, SEQ ID NO:54, SEQ ID NO:57, SEQ ID NO:60, SEQ ID NO:63, SEQ ID NO:66, SEQ ID NO:69 in that the amino acid sequence of the variant comprises 1 to 6 individual amino acid substitutions.

31. The polypeptide according to any one of claims 25 to 30, wherein said repeat region 1, repeat region 2 and repeat region 3 are according to any one of claims 2 to 23.

32. The polypeptide according to any one of the preceding claims, wherein said polypeptide has a beta trefoil fold structure.

33. The polypeptide according to any one of the preceding claims, wherein each of repeat region 1, repeat region 2 and repeat region 3 is capable of binding at least one disaccharide.

34. The polypeptide according to any one of the preceding claims, wherein each of repeat region 1, repeat region 2 and repeat region 3 is capable of binding at least one polysaccharide.

35. The polypeptide according to any one of the preceding claims, wherein the disaccharide or the polysaccharide comprises at least one Glc-β-1,6-Glc unit.

36. The polypeptide according to any one of the preceding claims, wherein the disaccharide or the polysaccharide comprises at least one Glc-β-1,2-Glc unit.

37. The polypeptide according to any one of the preceding claims, wherein the disaccharide or the polysaccharide comprises a glycan comprising at least one Glc-β-1,6-Glc unit.

38. The polypeptide according to any one of the preceding claims, wherein the disaccharide or the polysaccharide has a β-1,6-glucan backbone.

39. The polypeptide according to any one of the preceding claims, wherein the disaccharide or the polysaccharide has one or more Glc-β-1,6-Glc decoration.

40. The polypeptide according to any one of the preceding claims, wherein the disaccharide or the polysaccharide has one or more Glc-β-1,6-Glc decoration(s) on a β-1,3-glucan backbone.

41. The polypeptide according to any one of the preceding claims, wherein the disaccharide or the polysaccharide is scleroglucan.

42. The polypeptide according to any one of the preceding claims, wherein the disaccharide or the polysaccharide is pustulan.

43. The polypeptide according to any one of the preceding claims, wherein the saccharide is a polysaccharide.

44. The polypeptide according to any one of the preceding claims, wherein the polysaccharide is at least 10.000 Da, such as at least 50.000 Da, such as at least 100.000 Da, such as at least 500.000 Da, such as at least 1.000.000 Da, such as 2.000.000 Da.

45. The polypeptide according to any one of the preceding claims, wherein the disaccharide or the polysaccharide is water dispersible.

46. The polypeptide according to any one of the preceding claims, wherein the polysaccharide has a moisture content of at least 70 μg water per mg of saccharide, preferably at least 74 μg water per mg of saccharide, such as 77 μg water per mg of saccharide.

47. A polynucleotide encoding a polypeptide according to anyone of the preceding claims.

48. The polynucleotide according to claim 47, wherein said polynucleotide is codon-optimized for expression in a host cell.

49. A recombinant vector comprising a polynucleotide according to any one of claims 47 and 48, or a nucleic acid sequence encoding a carbohydrate-binding polypeptide according to any one of claims 1 to 46.

50. The vector according to claim 49, wherein the vector is a plasmid vector.

51. A host cell expressing the recombinant vector according to claim 50.

52. Use of at least one polypeptide according to any one of claims 1 to 46 for cross-linking two or more polysaccharides.

53. The use according to claim 52, wherein each polypeptide cross-links three polysaccharides.

54. A hydrogel comprising at least one polypeptide according to any one of claims 1 to 46 and two or more polysaccharides, wherein said hydrogel has a cross-linked structure.

55. The hydrogel according to claim 54, wherein said hydrogel is cross-linked with a bi- or multifunctional linker which is a polypeptide according to any one of claims 1 to 46.

56. The hydrogel according to any one of claims 54 to 55 wherein the polysaccharide comprises one or more β-1,3-glucan units with β-1,6-linked glucose decorations.

57. The hydrogel according to any one of claims 54 to 56, wherein the polysaccharide is scleroglucan.

58. The hydrogel according to any one of claims 54 to 57, wherein the hydrogel comprises at least 0.1 g/L polypeptide, such as at least 0.2 g/L polypeptide, such as at least 0.3 g/L polypeptide, such as at least 0.4 g/L polypeptide.

59. The hydrogel according to any one of claims 54 to 58, wherein the hydrogel comprises at least 2 g/L and at the most 10 g/L polysaccharide, preferably at least 3 g/L polysaccharide and at the most 5 g/L polysaccharide.

60. The hydrogel according to any one of claims 54 to 59, wherein the hydrogel comprises at least 2 g/L and at the most 10 g/L scleroglucan, preferably at least 3 g/L scleroglucan and at the most 5 g/L scleroglucan.

61. The hydrogel according to any one of claims 54 to 60, wherein the hydrogel comprises at least 90 wt. % water.

62. The hydrogel according to any one of claims 54 to 61, wherein the ratio of polypeptide to polysaccharide is between 1:20 and 1:2.

63. A cross-linker for crosslinking polysaccharides to form a hydrogel, wherein said cross-linker is a polypeptide according to any one of claims 1 to 46.

64. A method of manufacturing a hydrogel, the method comprising
a. providing a solution comprising a polysaccharide,
b. providing a solution comprising a polypeptide according to anyone of claims 1 to 46,
c. mixing the solutions under continuous stirring at room temperature, thereby obtaining a hydrogel.

65. The method of manufacturing a hydrogel according to claim 64, wherein the polysaccharide is according to anyone of claims 56 to 57.
